(19) United States
(12) Patent Application Publication
(10) Pub. No.: US 2007/0105867 A1
(43) Pub. Date: May 10, 2007

(54) Oral administration of N-(2-chloro-6-methylphenyl)-2-[[6-[4-(2-hydroxyethyl)-1-piperazinyl]-2-methyl-4-pyrimidinyl]amino]-1,3-thiazole-5-carboxamide and salts thereof

(75) Inventors: Ramakrishnan Chidambaram, Pennington, NJ (US); George M. Derbin, Robbinsville, NJ (US); Masaki Endo, Candiac (CA); Julia ZH Gao, Plainsboro, NJ (US); Tu Lee, Jhongli City (TW); Rajeshwar Motheram, Dayton, NJ (US); William Lawrence Parker, Pennington, NJ (US); Victor W. Rosso, East Windsor, NJ (US); Sailesh A. Varia, Princeton Junction, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/524,998

(22) Filed: Sep. 21, 2006

Related U.S. Application Data
(60) Provisional application No. 60/719,045, filed on Sep. 21, 2005.
(60) Provisional application No. 60/810,186, filed on Jun. 1, 2006.
(60) Provisional application No. 60/837,099, filed on Aug. 10, 2006.

(51) Int. Cl. A61K 31/506

(52) U.S. Cl. 514/252.19

(57) ABSTRACT

Disclosed are a method of treating cancer and/or other proliferative diseases comprising orally administering N-(2-chloro-6-methylphenyl)-2-((6-(4-(2-hydroxyethyl)-1-piperazinyl)-2-methyl-4-pyrimidinyl)amino)-1,3 -thiazole-5-carboxamide or a salt thereof, and pharmaceutical compositions comprising N-(2-chloro-6-methylphenyl)-2-((6-(4-(2-hydroxyethyl)-1-piperazinyl)-2-methyl-4 -pyrimidinyl)amino)-1,3-thiazole-5-carboxamide or a salt thereof. Also disclosed are N-(2-chloro-6-methylphenyl)-2-((6-(4-(2-hydroxyethyl)-1-piperazinyl)-2-methyl-4-pyrimidinyl)amino)-1,3-thiazole-5-carboxamide salts, as well as crystalline forms thereof.

### CROSS-REFERENCE TO RELATED APPLICATIONS

The present application claims the priority benefit of U.S. Provisional Application No. 60/719,045, filed Sep. 21, 2005; U.S. Provisional Application No. 60/810,186, filed Jun. 1, 2006, and U.S. Provisional Application No. 60/837,099, filed Aug. 10, 2006, each of which is incorporated herein by reference in its entirety.

### FIELD OF THE INVENTION

The present invention generally relates to a method of orally administering N-(2-chloro-6-methylphenyl)-2-((6-(4-(2-hydroxyethyl)-1-piperazinyl)-2-methyl-4-pyrimidinyl)amino)-1,3-thiazole-5-carboxamide or a salt thereof, and pharmaceutical compositions comprising N-(2-chloro-6-methylphenyl)-2-((6-(4-(2-hydroxyethyl)-1-piperazinyl)-2-methyl-4-pyrimidinyl)amino)-1,3-thiazole-5-carboxamide or a salt thereof. N-(2-chloro-6-methylphenyl)-2-((6-(4-(2-hydroxyethyl)-1-piperazinyl)-2-methyl-4-pyrimidinyl)amino)-1,3-thiazole-5-carboxamide is useful as an inhibitor of protein tyrosine kinases, such as Src kinase, and may be employed in the treatment of Src kinase-associated disorders such as cancer and/or other proliferative diseases.

### BACKGROUND OF THE INVENTION

The compound, N-(2-chloro-6-methylphenyl)-2-((6-(4-(2-hydroxyethyl)-1-piperazinyl)-2-methyl-4-pyrimidinyl)amino)-1,3-thiazole-5-carboxamide, has the structure of formula I: 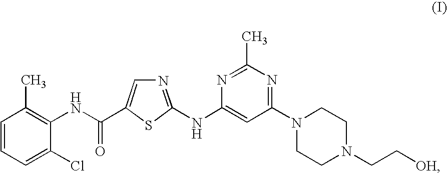
and is referred to herein as “Compound I”. Compound I, processes to prepare Compound I, and methods of treatment employing Compound I are disclosed in U.S. Pat. No. 6,596,746 B1; U.S. Patent Application Publication 2005/0215795; U.S. Patent Application Publication 2005/0009891; and U.S. Patent Application Publication 2006/0094728. These references are assigned to the present assignee and are incorporated herein by reference in their entirety.

Compound I is suitable for inhibiting Src kinase and is useful in the treatment of oncological diseases. However, before Compound I is used to treat diseases in patients, it is formulated into a pharmaceutical composition that can be administered to the patient; for example, into a dosage form suitable for oral, mucosal, parenteral, or transdermal administration. Formulations for oral administration are preferred since they are more convenient and easier to administer than other formulations. Also, the oral route of administration avoids the pain and discomfort of parenteral administration. Accordingly, formulations for oral administration are preferred by patients, and typically result in better patient compliance with dosing schedules.

The usefulness of an oral formulation, however, requires that the active agent be bioavailable and that the level of bioavailability does not vary widely. The bioavailability of orally administered drugs is often affected by various factors including, for example, the solubility of the drug in the gastrointestinal tract, the stability of the drug in the gastrointestinal tract, and drug absorption in the gastrointestinal tract. Further, these factors may be affected by coadministration of other drugs and/or the intake of food, which may lead to variability in the bioavailability of orally administered drug.

The aqueous solubility of Compound I is dependent on the pH of the aqueous medium. Compound I has higher solubility at a pH of 2 than at a pH of 5. In the oral administration of Compound I, the solubility and hence the bioavailability of Compound I can be affected by the pH of the stomach contents. The normal pH of the stomach is 1.2 to 1.8 according to C. J. Perigard, Clinical Analysis, Chapter 32, in Remington: The Science and Practice of Pharmacy 20^{th} Edition, A. R. Gennaro, editor; 2000, Lippinocott Williams & Wilkins, Baltimore, Md. However, during anticancer treatment, patients often take other medications to treat side effects or to ameliorate pain. Other medications may also be administered to treat medical conditions in oncology patients that are unrelated to cancer. For example, medications such as antacids or proton pump inhibitors can raise the pH of the stomach.

Typically, in preparing a pharmaceutical composition, a form of the active ingredient is sought that has a balance of desired properties, such as, for example, dissolution rate, solubility, bioavailability, and/or storage stability. For example, a sufficiently stable form of a sufficiently soluble and bioavailable form of the active ingredient is sought to prevent the sufficiently soluble and bioavailable form from converting during the manufacture and/or storage of the pharmaceutical composition to another form having an undesirable solubility and/or bioavailability profile. In addition, a form of the active ingredient may also be sought that permits the active ingredient to be isolated and/or purified during, for example, a preparative process.

Disclosed is a method of oral administration of Compound I or a salt thereof, which is useful for reducing the variability in the bioavailability of Compound I and/or increases the bioavailability of Compound I to the patient. Also, disclosed are pharmaceutical compositions comprising Compound I or a salt thereof, which is suitable for use in the aforementioned method of oral administration. Further, disclosed are salts of Compound I as well as one or more crystalline forms of these salts.

### SUMMARY OF THE INVENTION

Described herein is a pharmaceutical composition comprising:
a) Compound I of formula: 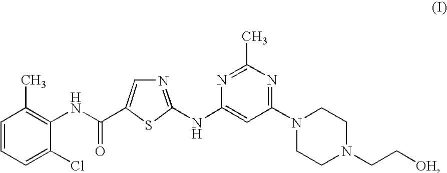
   and at least one acid pH modifier;
   and/or
b) a pharmaceutically-acceptable acid salt of Compound I and one or more pharmaceutically-acceptable excipients.

Described herein is a method of treating cancer in a human comprising: orally administering to said human:
a) a therapeutically effective amount of Compound I of formula: 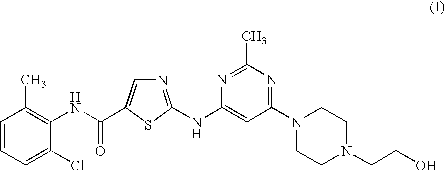
   and at least one acid pH modifier (Treatment A);
   and/or
b) a therapeutically effective amount of a pharmaceutically-acceptable acid salt of Compound I and one or more pharmaceutically-acceptable excipients (Treatment B).

Described herein are acid salts comprising Compound I: 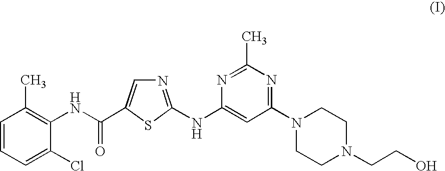
and at least one acid.

Described is a first crystalline form of a mono-hydrochloric acid salt of Compound I comprising Form CA-2.

Described is a second crystalline form of a mono-hydrochloric acid salt of Compound I comprising Form HAC2-1.

Described is a crystalline form of a di-hydrochloric acid salt of Compound I comprising Form H3-1.

Described is a first crystalline form of a monosulfuric acid salt of Compound I comprising Form SB-2.

Described is a second crystalline form of a monosulfuric acid salt of Compound I comprising Form SD-2.

Described is a first crystalline form of a hemisulfuric acid salt of Compound I comprising Form SA-1.

Described is a second crystalline form of a hemisulfuric acid salt of Compound I comprising Form SC-1.

Described is a crystalline form of an acetic acid salt of Compound I comprising Form NMP-1.

Described is a crystalline form of a phosphoric acid salt of Compound I comprising Form SA-1.

Described is a crystalline form of a hydrobromic acid salt of Compound I comprising Form H1.5-1.

Described is a crystalline form of a fumaric acid salt of Compound I comprising Form TO-1.

Described is a crystalline form of a salicylic acid salt of Compound I comprising Form SS-2.

Described is a crystalline form of a tartaric acid salt of Compound I.

Described is a crystalline form of a methanesulfonic acid salt of Compound I comprising Form PG-1.

Described is a first crystalline form of a maleic acid salt of Compound I comprising Form E-1.

Described is a second crystalline form of a maleic acid salt of Compound I comprising Form H3-2.

Described is a crystalline form of a p-toluenesulfonic acid salt of Compound I comprising Form N-1.

### BRIEF DESCRIPTION OF THE DRAWINGS

The invention is illustrated by reference to the accompanying figures described below.

FIG. 1 shows powder x-ray diffraction patterns (CuKαλ=1.5418 Å) of crystalline forms of the mono-hydrochloric acid salt of Compound I: observed PXRD for Form CA-2 at room temperature (FIG. 1.A); simulated PXRD for Form CA-2 (FIG. 1.B) at 25° C.; observed PXRD for Form HAC2-1 slurry at room temperature (FIG. 1.C); and simulated PXRD for Form HAC2-1 (FIG. 1.D), at −50° C.

FIG. 2 shows powder x-ray diffraction patterns (CuKαλ=1.5418 Å) of crystalline forms of the di-hydrochloric acid salts of Compound I: observed PXRD for Form I.5 slurry at room temperature (FIG. 2.A); observed PXRD for Form I.4 prepared by drying Form I.5 slurry, at room temperature(FIG. 2.B); observed PXRD for Form I.4 prepared by drying Form I.3 slurry, at room temperature (FIG. 2.C); and observed PXRD for Form I.3 slurry at room temperature (FIG. 2.D)

FIG. 3 shows powder x-ray diffraction patterns (CuKαλ=1.5418 Å) of a crystalline form of the di-hydrochloric acid salt of Compound I: observed PXRD for Form H3-1 at room temperature (FIG. 3.A) and simulated PXRD for Form H3-1 (FIG. 3.B), at −40° C.

FIG. 4 shows an observed powdered x-ray diffraction pattern (CuKαλ=1.5418 Å) of crystalline Form I.7 of the mono-hydrochloric acid salt of Compound I, at a room temperature.

FIG. 5 shows powder x-ray diffraction patterns (CuKαλ=1.5418 Å) of crystalline forms of sulfuric acid salts of Compound I: observed PXRD for Form II.3 slurry at room temperature (FIG. 5.A); observed PXRD for Form II.2 at room temperature (FIG. 5.B); observed PXRD for Form II.1 at room temperature (FIG. 5.C); and observed PXRD for Form II.4 at room temperature (FIG. 5.D).

FIG. 6 shows powder x-ray diffraction patterns (CuKαλ=1.5418 Å) of crystalline forms of sulfuric acid salts of Compound I: observed PXRD for Form II.6 at room temperature (FIG. 6.A) and observed PXRD for Form II.7 at room temperature (FIG. 6.B).

FIG. 7 shows powder x-ray diffraction patterns (CuKαλ=1.5418 Å) of crystalline forms of sulfuric acid salts of Compound I: observed PXRD for Form SD-2 slurry at room temperature (FIG. 7.A); simulated PXRD for Form SD-2 at room temperature (FIG. 7.B); observed PXRD for Form SC-1 slurry at room temperature (FIG. 7.C); and simulated PXRD for Form SC-1 at room temperature (FIG. 7.D).

FIG. 8 shows powder x-ray diffraction patterns (CuKαλ)=1.5418 Å) of crystalline forms of sulfuric acid salts of Compound I: observed PXRD for Form II.3 slurry, at room temperature (FIG. 8.A); observed PXRD for Form II.3 at room temperature (FIG. 8.B); observed PXRD for Form II.2 slurry at room temperature (FIG. 8.C); and observed PXRD for Form II.2 at room temperature (FIG. 8.D).

FIG. 9 shows powder x-ray diffraction patterns (CuKαλ=1.5418 Å) of crystalline forms of methanesulfonic acid salts of Compound I: observed PXRD for Form III.1 at room temperature (FIG. 9.A) and observed PXRD for Form III.2 at room temperature (FIG. 9.B).

FIG. 10 shows powder x-ray diffraction patterns (CuKαλ=1.5418 Å) of crystalline forms of methanesulfonic acid salts of Compound I: observed PXRD for Form III.3 at room temperature (FIG. 10.A); observed PXRD for Form III.4 at room temperature (FIG. 10.B); observed PXRD for Form III.5 at room temperature (FIG. 10.C); and observed PXRD for Form III.6 at room temperature (FIG. 10.D).

FIG. 11 shows a powder x-ray diffraction pattern (CuKαλ=1.5418 Å) of a crystalline form of a methanesulfonic acid salt of Compound I: observed PXRD for Form PG-1 at room temperature (FIG. 11.A) and simulated PXRD for Form PG-1 at −50° C. (FIG. 11.B).

FIG. 12 shows powder x-ray diffraction patterns (CuKαλ=1.5418 Å) of crystalline forms of phosphoric acid salts of Compound I: observed PXRD for Form IV.1 at room temperature (FIG. 12.A); observed PXRD for Form IV.2 at room temperature (FIG. 12.B); and observed PXRD for Form IV.3 at room temperature (FIG. 12.C).

FIG. 13 shows powder x-ray diffraction patterns (CuKαλ=1.5418 Å) of crystalline forms of phosphoric acid salts of Compound I: observed PXRD for Form IV.4 at room temperature (FIG. 13.A); observed PXRD for Form IV.6 at room temperature (FIG. 13.B); observed PXRD for Form IV.5 at room temperature (FIG. 13.C); and observed PXRD for Form IV.7 at room temperature (FIG. 13.D).

FIG. 14 shows powder x-ray diffraction patterns (CuKαλ=1.5418 Å) of crystalline forms of phosphoric acid salts of Compound I: simulated PXRD for Form SA-1 at −60° C. (FIG. 14.A); observed PXRD for Form IV.9 at room temperature (FIG. 14.B); and observed PXRD for Form IV.8 at room temperature (FIG. 14.C).

FIG. 15 shows powder x-ray diffraction patterns (CuKαλ=1.5418 Å) of crystalline forms of tartaric acid salts of Compound I: observed PXRD for L-tartaric acid salt Form V.2 at room temperature (FIG. 15.A); observed PXRD for D-tartaric acid salt Form V.1 at room temperature (FIG. 15.B); and observed PXRD for racemic tartaric acid salt Form V.3 at room temperature (FIG. 15.C).

FIG. 16 shows powder x-ray diffraction patterns (CuKαλ=1.5418 Å) of crystalline forms of L-tartaric acid salts of Compound I: observed PXRD for Form V.1 at room temperature (FIG. 16.A) and simulated PXRD for Form V.1 (FIG. 16.B), at −50° C.

FIG. 17 shows powder x-ray diffraction patterns (CuKαλ=1.5418 Å) of crystalline forms of benzoic acid salts of Compound I: observed PXRD for Form VI.1 at room temperature (FIG. 17.A) and observed PXRD for Form VI.2 slurry at room temperature (FIG. 17.B).

FIG. 18 shows powder x-ray diffraction patterns (CuKαλ=1.5418 Å) of crystalline forms of fumaric acid salts of Compound I: observed PXRD for Form VII.1 at room temperature (FIG. 18.A); observed PXRD for Form VII.2 at room temperature (FIG. 18.B); observed PXRD for Form VII.3 at room temperature (FIG. 18.C); observed PXRD for Form VII.4 at room temperature (FIG. 18.D); and observed PXRD for Form VII.5 (FIG. 18.E), at room temperature.

FIG. 19 shows powder x-ray diffraction patterns (CuKαλ=1.5418 Å) of crystalline forms of fumaric acid salts of Compound I: observed PXRD for Form VII.5 at room temperature (FIG. 19.A); observed PXRD for mixture of Form VII.5 and Form VII.6 slurry at room temperature (FIG. 19.B); and simulated PXRD for Form VII.6 (FIG. 19.C), at −25° C.

FIG. 20 shows powder x-ray diffraction patterns (CuKαλ=1.5418 Å) of crystalline forms of hemi-fumaric acid salts of Compound I: observed PXRD for Form VII.7 at room temperature (FIG. 20.A); observed PXRD for Form VII.8 at room temperature (FIG. 20.B); observed PXRD for Form VII.9 at room temperature (FIG. 20.C); and observed PXRD for Form VII.10 wet cake at room temperature(FIG. 20.D).

FIG. 21 shows powder x-ray diffraction patterns (CuKαλ=1.5418 Å) of crystalline forms of hemi-fumaric acid salts of Compound I: observed PXRD for Form VII.11 at room temperature (FIG. 21.A); observed PXRD for Form VII.8 at room temperature (FIG. 21.B); observed PXRD for Form VII.12 at room temperature (FIG. 21.C); and observed PXRD for Form VII.13 slurry at room temperature (FIG. 21.D).

FIG. 22 shows powder x-ray diffraction patterns (CuKαλ=1.5418 Å) of crystalline forms of maleic acid salts of Compound I: observed PXRD for Form VIII.1 at room temperature (FIG. 22.A); observed PXRD for Form VIII.2 slurry (FIG. 22.B), at room temperature; and simulated PXRD for Form E-1 (FIG. 22.C) at −50° C.

FIG. 23 shows powder x-ray diffraction patterns (CuKαλ=1.5418 Å) of crystalline forms of maleic acid salts of Compound I: observed PXRD for Form VIII.4 at room temperature (FIG. 23.A); observed PXRD for Form VIII.5 slurry at room temperature (FIG. 23.B); observed PXRD for Form H3-2 slurry at room temperature (FIG. 23.C); and simulated PXRD for Form H3-2 at −70° C. (FIG. 22.D).

FIG. 24 shows powder x-ray diffraction patterns (CuKαλ=1.5418 Å) of crystalline forms of hemi-maleic acid salts of Compound I: observed PXRD for Form VIII.7 at room temperature (FIG. 24.A) and observed PXRD for Form VIII.8 slurry at room temperature (FIG. 24.B).

FIG. 25 shows powder x-ray diffraction patterns (CuKαλ=1.5418 Å) of a crystalline form of a hydrobromic acid salt of Compound I: observed PXRD for Form H1.5-1 at room temperature (FIG. 25.A) and simulated PXRD for Form H1.5-1 (FIG. 25.B) at −50° C.

FIG. 26 shows powder x-ray diffraction patterns (CuKαλ=1.5418 Å) of crystalline forms of benzenesulfonic acid salts of Compound I: observed PXRD for Form XI.1 at room temperature (FIG. 26.A); observed PXRD for Form XI.3 at room temperature (FIG. 26.B); and observed PXRD for Form XI.2 slurry at room temperature (FIG. 26.C).

FIG. 27 shows a powder x-ray diffraction pattern (CuKαλ=1.5418 Å) of a crystalline form of a citric acid salt of Compound I: observed PXRD for Form XII.1, at room temperature.

FIG. 28 shows powder x-ray diffraction patterns (CuKαλ=1.5418 Å) of crystalline forms of acetic acid salts of Compound I: observed PXRD for Form XIII.1 at room temperature (FIG. 28.A) and simulated PXRD for Form NMP-1 (FIG. 28.B), at 25° C.

FIG. 29 shows powder x-ray diffraction patterns (CuKαλ=1.5418 Å) of crystalline forms of p-toluenesulfonic acid salts of Compound I: observed PXRD for Form XIV.1 at room temperature (FIG. 29.A); observed PXRD for Form XIV.2 at room temperature (FIG. 29.B); and simulated PXRD for Form XIV.2 at room temperature (FIG. 29.C).

FIG. 30 shows powder x-ray diffraction patterns (CuKαλ=1.5418 Å) of crystalline forms of gentisic acid salts of Compound I: observed PXRD for Form XV.2 at room temperature (FIG. 30.A) and observed PXRD for Form XV.1 at room temperature (FIG. 30.B).

FIG. 31 shows powder x-ray diffraction patterns (CuKαλ=1.5418 Å) of a crystalline form of a salicylic acid salt of Compound I: observed PXRD for Form SS-2 at room temperature (FIG. 31.A) and simulated PXRD for Form SS-2 (FIG. 31.B) at a temperature of −40° C.

FIG. 32 shows a powder x-ray diffraction pattern (CuKαλ=1.5418 Å) of a crystalline form of a p-acetamido benzoic acid salt of Compound I: observed PXRD for Form XVII.1 at room temperature.

FIG. 33 shows a powder x-ray diffraction pattern (CuKαλ=1.5418 Å) of a crystalline form of a L-malic acid salt of Compound I: observed PXRD for Form IX.1 at room temperature.

FIG. 34 shows a powder x-ray diffraction pattern (CuKαλ=1.5418 Å) of crystalline forms of sulfuric acid salts of Compound I: simulated PXRD for Form SA-1 at room temperature (FIG. 34.A); and simulated PXRD for Form SB-2 at room temperature (FIG. 34.B).

FIG. 35 shows a simulated (bottom) (calculated from atomic coordinates generated at room temperature) and experimental (top) PXRD patterns for the crystalline monohydrate of Compound I, measured at a temperature of about 25° C.

### DETAILED DESCRIPTION OF THE INVENTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

The names used herein to characterize a specific form, e.g. “H3-1” etc., should not be considered limiting with respect to any other substance possessing similar or identical physical and chemical characteristics, but rather it should be understood that these designations are mere identifiers that should be interpreted according to the characterization information also presented herein.

The definitions set forth herein take precedence over definitions set forth in any patent, patent application, and/or patent application publication incorporated herein by reference.

All numbers expressing quantities of ingredients, weight percentages, temperatures, and so forth that are preceded by the word “about” are to be understood as only approximations so that slight variations above and below the stated number may be used to achieve substantially the same results as the stated number. Accordingly, unless indicated to the contrary, numerical parameters preceded by the word “about” are approximations that may vary depending upon the desired properties sought to be obtained. At the very least, each numerical parameter should at least be construed in light of the number or reported significant digits and by applying ordinary rounding techniques.

As used herein, “polymorphs” refer to crystalline forms having the same chemical compositions but different spatial arrangements of the molecules and/or ions forming the crystals.

As used herein, “amorphous” refers to a solid form of a molecule and/or ions that are not crystalline. An amorphous solid does not display a definitive X-ray diffraction pattern with sharp maxima.

As used herein, “slurry” refers to a saturated solution of Compound I and an additional amount of Compound I to give a heterogeneous solution of Compound I and at least one solvent.

As used herein, the term “mole equivalent” refers to the number of equivalents of a compound on a mole basis. For example, a salt comprising 1 mole equivalent (eq.) of HCl and 1 mole equivalent of Compound I has a ratio of 1 mole of HCl for each mole of Compound I. In another example, a salt comprising 1 mole equivalent of H₃PO₄ to 1 mole equivalent of Compound I has a ratio of 1 mole of H₃PO₄ for each mole of Compound I.

As used herein, “substantially pure,” when used in reference to an acid salt of Compound I means a sample of the Compound I acid salt having a purity greater than 90 weight %, including greater than 91, 92, 93, 94, 95, 96, 97, 98, and 99 weight %, and also including equal to about 100 weight % of the Compound I acid salt, based on the weight of the Compound I acid salt together with reaction impurities and/or processing impurities arising from its preparation. For example, a sample of a Compound I acid salt may be deemed substantially pure in that it has a purity greater than 90 weight % of the Compound I acid salt, as measured by means that are at this time known and generally accepted in the art, where the remaining less than 10 weight % of material comprises other Compound I salts, other salts, Compound I, and/or reaction impurities and/or processing impurities. The presence of reaction impurities and/or processing impurities may be determined by analytical techniques known in the art, such as, for example, chromatography, nuclear magnetic resonance spectroscopy, mass spectrometry, or infrared spectroscopy.

As used herein, “substantially pure,” when used in reference to a crystalline form, means a sample of the crystalline form of the Compound I acid salt having a purity greater than 90 weight %, including greater than 91, 92, 93, 94, 95, 96, 97, 98, and 99 weight %, and also including equal to about 100 weight % of the crystalline form of the Compound I acid salt, based on the weight of the Compound I acid salt. The remaining material comprises other form(s) of the Compound I acid salt. For example, a crystalline form of a Compound I salt may be deemed substantially pure in that it has a purity greater than 90 weight % of the crystalline form of the Compound I salt, as measured by means that are at this time known and generally accepted in the art, where the remaining less than 10 weight % of material comprises other form(s) of the Compound I acid salt.

As used herein, the parameter “molecules/asymmetric unit” refers to the number of molecules of Compound I in the asymmetric unit.

As used herein, the unit cell parameter “molecules/unit cell” refers to the number of molecules of Compound I in the unit cell.

As used herein, “V” represents the volume of the unit cell, “Z” represents the number of molecules per unit cell, and “V/Z” represents volume per molecule in the unit cell.

When dissolved, the crystalline form of a salt of Compound I loses its crystalline structure, and is therefore referred to as a solution of Compound I salt.

One or more of the crystalline forms of a salt of Compound I disclosed herein, may be used for the preparation of liquid formulations in which the compound salt is dissolved or suspended.

The crystalline forms may have voids or channels within their crystalline structures. The voids or channels may optionally be filled, either partially or completely, with solvent or a mixture of solvents. The solvent contained within these voids or channels may be ordered, partly ordered, or disordered. Certain crystalline forms may comprise a void or channel of sufficient size or of a particular shape that allows the void or channel to contain a number of different types of solvents without significantly affecting the unit cell parameters of the crystalline form.

The term “therapeutically effective amount” means an amount that, when administered alone or an amount when administered with an additional therapeutic agent, is effective to prevent, suppress, or ameliorate the disease of condition or the progression of the disease or condition.

One aspect of the invention is related to a pharmaceutical composition comprising: a) Compound I and at least one acid pH modifier; and/or b) a pharmaceutically-acceptable acid salt of Compound I and one or more pharmaceutically-acceptable excipients.

In one embodiment, the pharmaceutical composition comprises Compound I and at least one acid modifier. The acid pH modifier is a material capable of lowering the pH of gastric contents with a higher than normal pH value, such as a pH of greater than about 4. One or more different acid pH modifiers may be used. Preferably, the at least one acid pH modifier provides the microclimate of the formulation with a pH less than about 4, more preferably less than about 3, and most preferably less than about 2.5. For example, the ingestion of the at least one acid pH modifier provides the microclimate of the formulation with a pH in the range of from about 1 to about 4, preferably in the range of from about 1 to about 3, more preferably in the range of from about 1 to about 2.5, and most preferably in the normal pH range of the stomach, such as a pH in the range of from about 1.2 to about 1.8. Examples of suitable acid pH modifiers include, but are not limited to, citric acid, tartaric acids, maleic acid, fumaric acid, phosphoric acid, lactic acid, succinic acid, acetic acid, ascorbic acid, aspartic acid, hydrochloric acid, and glutamic acid. In this embodiment, the pharmaceutical composition comprises an amount of the at least one acid pH modifier that is sufficient, upon oral administration, to provide the stomach and/or the microclimate of the formulation with a pH that is suitable for reducing the variability in the bioavailability of Compound I and/or increasing the bioavailability of Compound I. Examples of suitable amounts for the at least one acid pH modifier in the pharmaceutical composition include about 1 to about 50 weight %, and from about 2 to about 25 weight % of the at least one acid pH modifier, based on the weight of the oral dosage. Other examples of suitable amounts for the at least one acid pH modifier include molar ratios of the at least one acid pH modifier to Compound I in the range of from about 0.1:1 to about 20:1, and in the range of from about 0.5:1 to about 10:1. The pharmaceutical composition of this embodiment may be provided as a single dosage form comprising Compound I and at least one acid pH modifier; or alternatively, as two separate dosage forms in which one dosage form comprises the at least one acid pH modifier and the other dosage form comprises Compound I. Further, the pharmaceutical composition of this embodiment may optionally comprise one or more pharmaceutically-acceptable excipients. Examples of pharmaceutically-acceptable excipients include, but are not limited to, binders, fillers, disintegrating agents, other pH modifiers, lubricants, solid diluents, liquid diluents such as water, oils, and alcohols, granulating agents, glidants, preservatives, antioxidants, coloring agents, flavoring agents, and surface active agents. The pharmaceutical composition of this embodiment may optionally comprise a pharmaceutically-acceptable acid salt of Compound I in addition to Compound I.

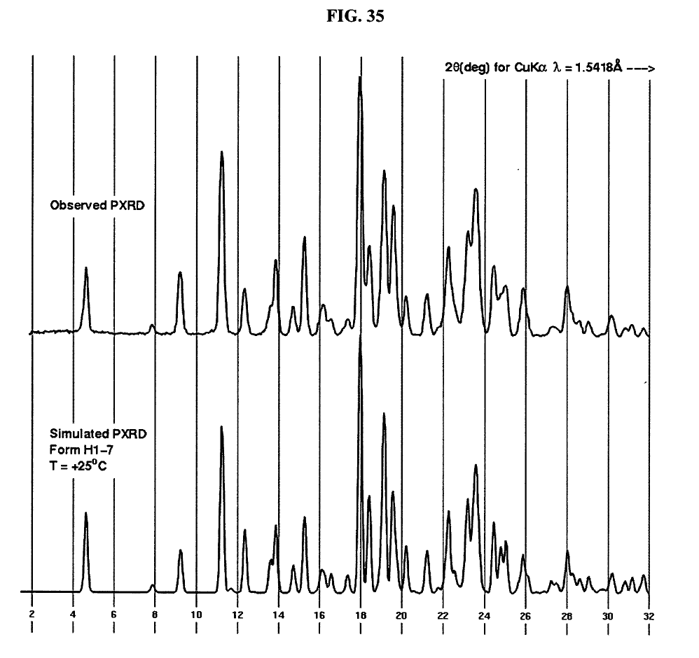

In the disclosed pharmaceutical composition, Compound I may be provided as a crystalline material, such as a monohydrate crystalline form. Preferably, the crystalline form of Compound I is in substantially pure form. In one embodiment, the pharmaceutical composition comprising Compound I and at least one acid pH modifier wherein Compound I is provided as a monohydrate crystalline form characterized by the simulated powder x-ray diffraction pattern substantially in accordance with that shown in FIG. 35(bottom) and/or the observed powder x-ray diffraction pattern substantially in accordance with that shown in FIG. 35(top) and wherein the monohydrate crystalline form is in substantially pure form.

In a different embodiment, the pharmaceutical composition comprises a pharmaceutically-acceptable acid salt of Compound I and one or more pharmaceutically-acceptable excipients. Examples of pharmaceutically-acceptable excipients include, but are not limited to, binders, fillers, disintegrating agents, pH modifiers such as acid pH modifiers, lubricants, solid diluents, liquid diluents such as water, oils, and alcohols, granulating agents, glidants, preservatives, antioxidants, coloring agents, flavoring agents, and surface active agents. Two or more different acid salts of Compound I may be used in the pharmaceutical composition of this embodiment. Additionally, the pharmaceutical composition of this embodiment may optionally comprise Compound I in addition to the pharmaceutically-acceptable acid salt of Compound I. Further, the acid salt of Compound I may be provided as a crystalline material, which preferably is in substantially pure form.

Another aspect of the present invention is related to a method of treating cancer in a human, comprising: orally administering to the human: a) a therapeutically effective amount of Compound I and at least one acid pH modifier referred to herein as “Treatment A”; and/or b) a therapeutically effective amount of a pharmaceutically-acceptable salt of Compound I and one or more pharmaceutically-acceptable excipients, referred to herein as “Treatment B”. Various cancers may be treated with the present method including, but are not limited to, gastrointestinal stromal tumor (GIST) or a leukemia selected from chronic myelogenous leukemia (CML), acute lymphoblastic leukemia (ALL), Philadelphia chromosome positive acute lymphoblastic leukemia (Ph+ ALL), and acute myelogenous leukemia.

In one embodiment, the method of treatment is used to treat a human who is administered one or more medicines that raise the pH of the stomach of the human prior to or during administration of Treatment A and/or Treatment B. Examples of such medicines include antacids and proton pump inhibitors such as omeprazole, esomeprazole, lansoprazole, pantoprazole, and rabeprazole sodium.

Another embodiment provides a method of treatment comprising orally administering to the human Treatment A and/or Treatment B, wherein the administration of Treatment A and/or Treatment B provides enhanced bioavailability of Compound I as compared with when Compound I is administered unaccompanied by the at least one acid pH modifier.

The acid salt of Compound I comprises a salt of Compound I and at least one acid. The acid salt may be formed by various reactions including, for example, combining Compound I and at least one acid, combining Compound I with a different acid salt, combining an acid salt of Compound I with a different acid; or combining an acid salt of Compound I with a different acid salt. In one embodiment, the at least one acid in the acid salt of Compound I is: p-acetamidobenzoic acid, acetic acid, benzoic acid, benzenesulfonic acid, citric acid, fumaric acid, gentisic acid, hydrobromic acid, hydrochloric acid, maleic acid, malic acid, methanesulfonic acid, phosphoric acid, salicylic acid, sulfuric acid, D-tartaric acid, L-tartaric acid, or p-toluenesulfonic acid. Preferably, the at least one acid is fumaric acid, hydrobromic acid, methanesulfonic acid, phosphoric acid, salicylic acid, sulfuric acid, tartaric acid, or p-toluenesulfonic acid.

The acid salt of Compound I may optionally comprise water and/or one or more solvents, such as, for example, methanol and acetic acid. The water and/or the one or more solvents may be present in a stoichiometric amount, for example, as a hemi-hydrate wherein the Compound I acid salt comprises 0.5 mole of water for each mole of Compound I.

Hydrochloric Acid Salts of Compound I

Hydrochloric acid (HCl) salts of Compound I include, for example, mono-HCl salts, which have a ratio of one mole of HCl to one mole of Compound I; and di-HCl salts, which have a ratio of two moles of HCl to one mole of Compound I. The HCl salts of Compound I may optionally comprise water and/or one or more solvents, such as, for example, ethanol and acetic acid.

In one embodiment, the HCl salt of Compound I is provided as a mono-HCl salt. Preferably, the mono-HCl salt is substantially pure. Further, the mono-HCl salt may be provided as crystalline material. Examples of crystalline forms of the mono-HCl salt of Compound I include a first crystalline form comprising Form CA-2 and a second crystalline form comprising Form HAC2-1.

The first crystalline form of a mono-HCl salt of Compound I comprises one mole of HCl for each mole of Compound I, and may optionally comprise solvent. This crystalline form is referred to herein as “Form CA-2” or “Form I.1”. The crystalline structure of Form CA-2 includes a cavity or channel, which may be partially or fully occupied by solvent or a mixture of solvents. Examples of suitable solvents include, but are not limited to, alcohols such as methanol and ethanol, and water. The processes to prepare and isolate Form CA-2 as well as the drying and storage conditions will generally determine the types and amounts of the solvents found in this crystalline form. For example, Form CA-2 can be prepared comprising 0.4 mole of ethanol and 0.8 mole of water.

In one embodiment, Form CA-2 of the mono-HCl salt of Compound I is characterized by unit cell parameters substantially equal to the following:
Cell dimensions:
   a=6.55 Å
   b=20.70 Å
   c=20.91 Å
   α=90 degrees
   β=91.0 degrees
   γ=90 degrees
Space group: P2₁/c
Molecules/unit cell: 4
V/Z=708 Å³
   wherein measurement of said crystalline form is at a temperature of about 25° C.

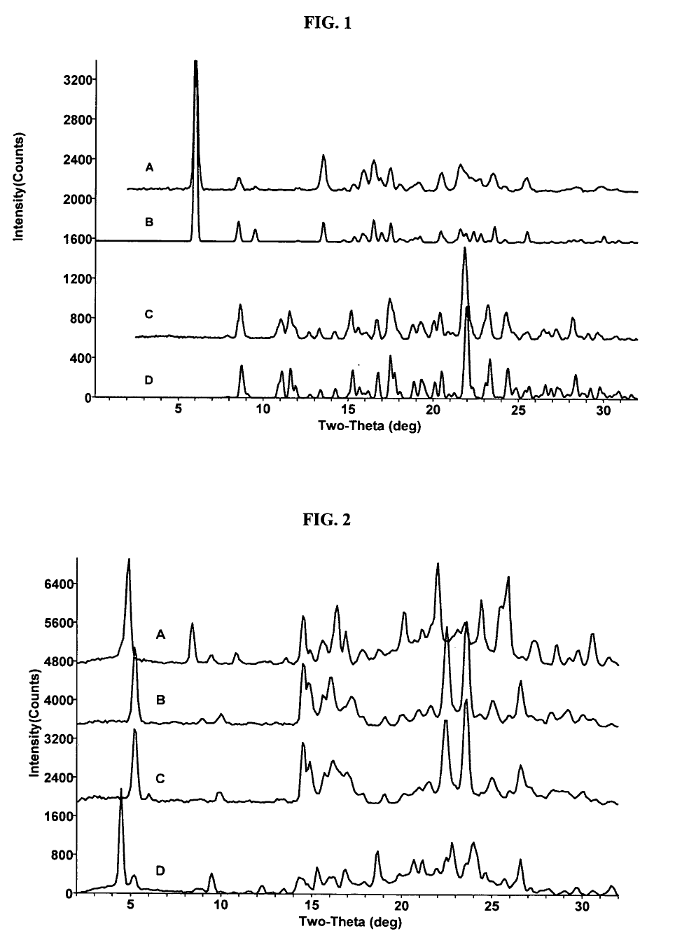

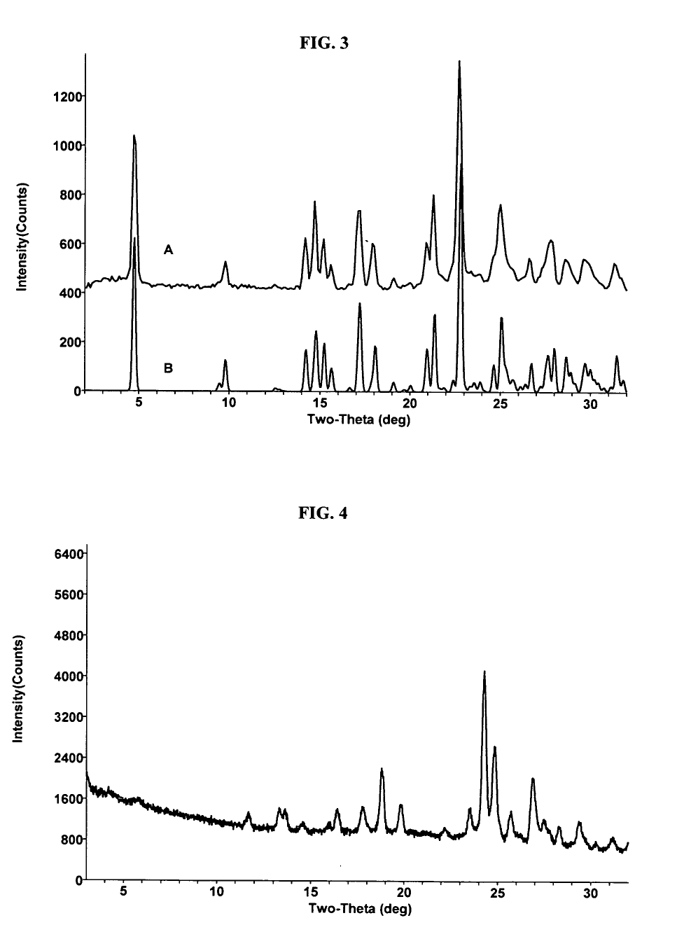

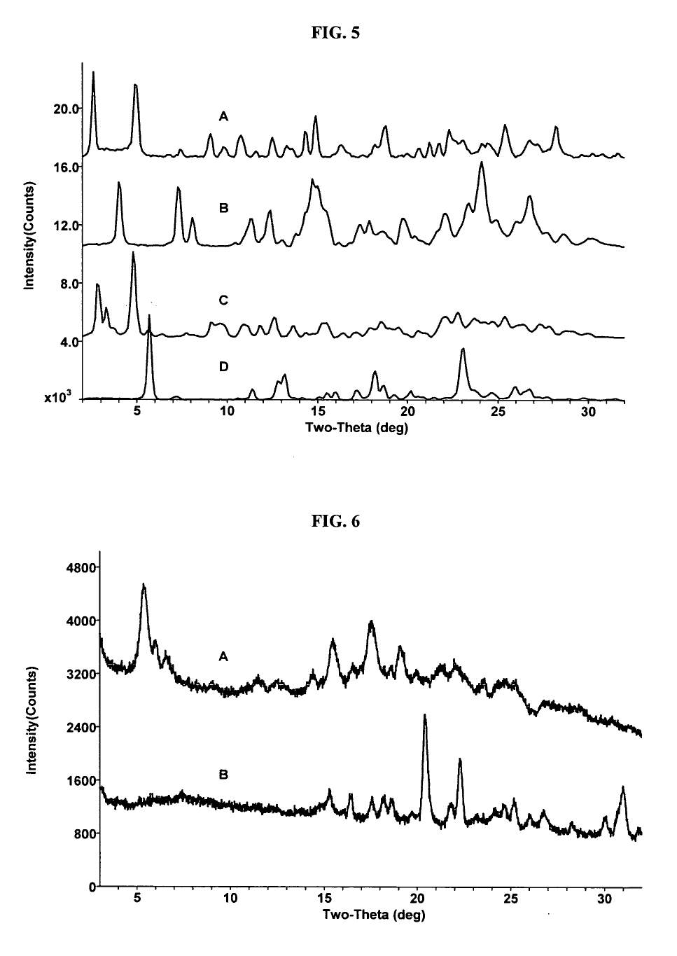

In a different embodiment, the Form CA-2 of the mono-HCl salt of Compound I is characterized by the simulated powder x-ray diffraction pattern substantially in accordance with that shown in FIG. 1.B and/or by the observed powder x-ray diffraction pattern substantially in accordance with that shown in FIG. 1.A.

In a different embodiment, the Form CA-2 of the mono-HCl salt of Compound I is characterized by a powder x-ray diffraction pattern (CuKαλ=1.5418 Å) comprising four or more 2θ values, preferably comprising five or more 2θ values, selected from: 6.0±0.1, 9.5±0.1, 13.5±0.1, 16.5±0.1, 16.9±0.1, and 23.6±0.1, wherein measurement of the crystalline form is at a temperature of about 25° C.

In still another embodiment, the Form CA-2 of the mono-HCl salt of Compound I is provided in substantially pure form.

The second crystalline form of a mono-HCl salt of Compound I comprises one mole of HCl for each mole of Compound I, and further comprises up to 2 moles of acetic acid. This crystalline form is referred to herein as “Form HAC2-1” or “Form I.2”.

In one embodiment, Form HAC2-1 of the mono-HCl salt of Compound I is characterized by unit cell parameters approximately equal to the following:
Cell dimensions:
   a=19.98 Å
   b=13.89 Å
   c =22.24 Å
   α=90 degrees
   β=90 degrees
   γ=90 degrees
Space group: Pbca
Molecules/unit cell: 8
V/Z=771 Å³
Density (calculated)=1.387 g/cm³
   wherein measurement of said crystalline form is at a temperature of about −50° C.

In a different embodiment, the Form HAC2-1 of the mono-HCl salt of Compound I is characterized by the simulated powder x-ray diffraction pattern substantially in accordance with that shown in FIG. 1.D and/or by the observed powder x-ray diffraction pattern substantially in accordance with that shown in FIG. 1.C (slurry).

In a different embodiment, the Form HAC2-1 of the mono-HCl salt of Compound I is characterized by a powder x-ray diffraction pattern (CuKαλ=1.5418 Å) comprising four or more 2θ values, preferably comprising five or more 2θ values, selected from: 8.7±0.1, 11.1±0.1, 11.6±0.1, 15.2±0.1, 17.4±0.1, 20.4±0.1, 21.8±0.1, 23.2±0.1, and 24.2±0.1, wherein measurement of the crystalline form is at a temperature of about 25° C.

In still another embodiment, the Form HAC2-1 of the mono-HCl salt of Compound I is provided in substantially pure form.

In a different embodiment, the HCl salt of Compound I is provided as a di-HCl salt. Preferably, the di-HCl salt is substantially pure. Further, the di-HCl salt may be provided as crystalline material. An example of a crystalline form of the di-HCl salt of Compound I include a crystalline form comprising Form H3-1.

A crystalline form of a di-HCl salt of Compound I comprises two moles of HCl for each mole of Compound I, and further comprises up to three moles of water. This crystalline form is referred to herein as “Form H3-1” or “Form I.6.

In one embodiment, Form H3-1 of the di-HCl salt of Compound I is characterized by unit cell parameters substantially equal to the following:
Cell dimensions:
   a=7.45 Å
   b=10.29 Å
   c=18.69 Å
   α=90 degrees
   β=93.2 degrees
   γ=90 degrees
Space group: P2₁
Molecules/unit cell: 2
V/Z=716 Å³
Density (calculated)=1.426 g/cm³
   wherein measurement of said crystalline form is at a temperature of about −40° C.

In a different embodiment, the Form H3-1 of the di-HCl salt of Compound I is characterized by the simulated powder x-ray diffraction pattern substantially in accordance with that shown in FIG. 3.B and/or by the observed powder x-ray diffraction pattern substantially in accordance with that shown in FIG. 3.A.

In a different embodiment, the Form H3-1 of the di-HCl salt of Compound I is characterized by a powder x-ray diffraction pattern (CuKαλ=1.5418 Å) comprising four or more 2θ values, preferably comprising five or more 2θ values, selected from: 4.7±0.1, 14.2±0.1, 14.7±0.1, 15.2±0.1, 15.6±0.1, 17.2±0.1, 21.3±0.1, 22.7±0.1, and 25.0±0.1, wherein measurement of the crystalline form is at a temperature of about 25° C.

In still another embodiment, the Form H3-1 of the di-HCl salt of Compound I is provided in substantially pure form.

Sulfuric Acid Salts of Compound I

Sulfuric acid (H₂SO₄) salts of Compound I include, for example, hemi-H₂SO₄ salts, which have a ratio of 0.5 moles of H₂SO₄ to one mole of Compound I, and mono-H₂SO₄ salts, which have a ratio of one mole of H₂SO₄ to one mole of Compound I. The H₂SO₄ salts of Compound I may optionally comprise water and/or other solvents.

In one embodiment, the H₂SO₄ salt of Compound I is provided as a mono-H₂SO₄ salt. Preferably, the mono-H₂SO₄ salt is substantially pure. Further, the mono-H₂SO₄ salt may be provided as crystalline material. Examples of crystalline forms of the mono-H₂SO₄ salt of Compound I include a first crystalline form comprising Form SB-2 and a second crystalline form comprising Form SD-2.

The first crystalline form of a mono-H₂SO₄ salt of Compound I comprises one mole of H₂SO₄ for each mole of Compound I, and further comprises water and tetrahydrofuran. This crystalline form is referred to herein as “Form SB-2” or “Form 11.5”.

In one embodiment, Form SB-2 of the mono-H₂SO₄ salt of Compound I is characterized by unit cell parameters substantially equal to the following:
Cell dimensions:
   a=8.07 Å
   b=31.76 Å
   c=12.54 Å
   α=90 degrees
   β=94.56 degrees
   γ=90 degrees
Space group: P2₁/n
Molecules/unit cell: 4
Volume=801 Å³
   wherein measurement of said crystalline form is at a temperature of about 25° C.

In a different embodiment, the Form SB-2 of the mono-H₂SO₄ salt of Compound I is characterized by the simulated powder x-ray diffraction pattern substantially in accordance with that shown in FIG. 34.B.

In still another embodiment, the Form SB-2 of the mono-H₂SO₄ salt of Compound I is provided in substantially pure form.

The second crystalline form of a mono-H₂SO₄ salt of Compound I comprises one mole of H₂SO₄ for each mole of Compound I, and further comprises water and 1-methyl-2-pyrrolidinone. This crystalline form is referred to herein as “Form SD-2” or “Form II.10”.

In one embodiment, Form SD-2 of the mono-H₂SO₄ salt of Compound I is characterized by unit cell parameters substantially equal to the following:
Cell dimensions:
   a=8.12 Å
   b=31.79 Å
   c=12.65 Å
   α=90 degrees
   β=92.16 degrees
   γ=90 degrees
Space group: P2₁/n
Molecules/unit cell: 4
Volume=815 Å³
   wherein measurement of said crystalline form is at a temperature of about 25° C.

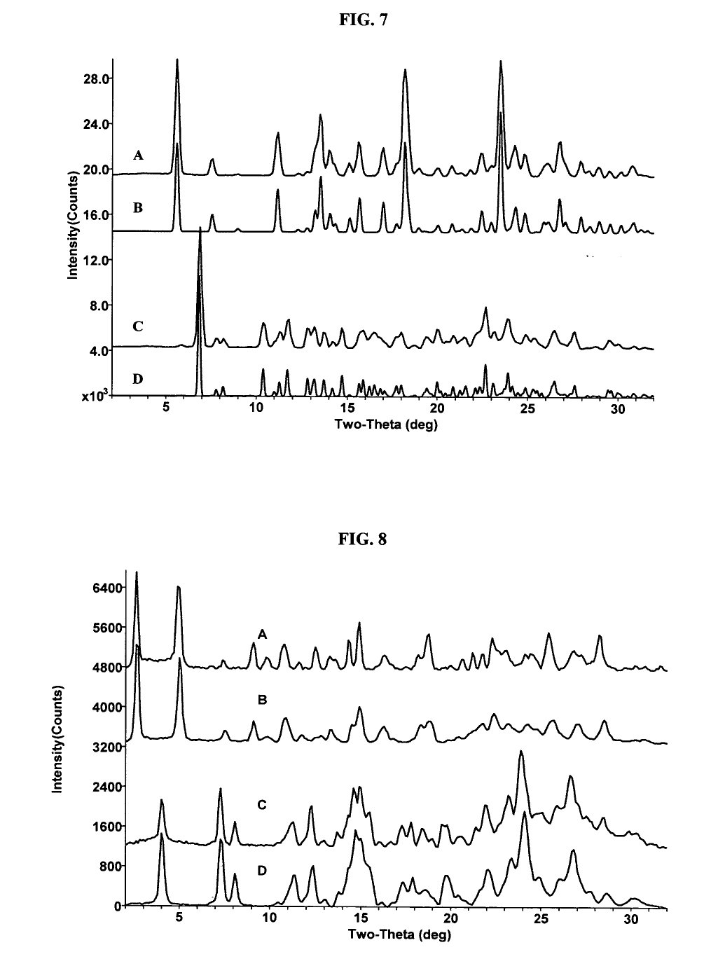

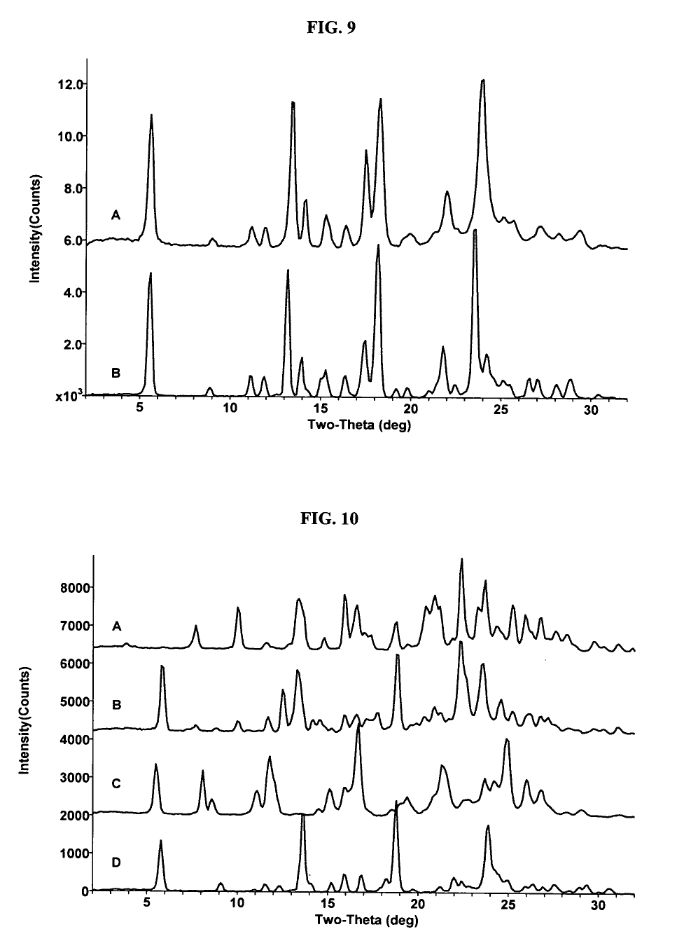

In a different embodiment, the Form SD-2 of the mono-H₂SO₄ salt of Compound I is characterized by the simulated powder x-ray diffraction pattern substantially in accordance with that shown in FIG. 7.B and/or by the observed powder x-ray diffraction pattern substantially in accordance with that shown in FIG. 7.A.

In a different embodiment, the Form SD-2 of the mono-H₂SO₄ salt of Compound I is characterized by a powder x-ray diffraction pattern (CuKαλ=1.5418 Å) comprising four or more 2θ values, preferably comprising five or more 2θ values, selected from: 5.6±0.1, 7.5±0.1, 13.5±0.1, 14.0±0.1, 18.2±0.1, 23.5±0.1, 26.7±0.1, and 27.9±0.1, wherein measurement of the crystalline form is at a temperature of about 25° C.

In still another embodiment, the Form SD-2 of the mono-H2SO4 salt of Compound I is provided in substantially pure form.

The first crystalline form of a hemi-H₂SO₄ salt of Compound I comprises 0.5 mole of H₂SO₄ for each mole of Compound I, further comprises up to about 0.5 mole of water for each molecule of Compound I, and may also comprise ethanol. This crystalline form is referred to herein as “Form SA-1” or “Form II.8”.

In one embodiment, Form SA-1 of the hemi-H₂SO₄ salt of Compound I is characterized by unit cell parameters substantially equal to the following:
Cell dimensions:
   a=17.02 Å
   b=16.96 Å
   c=20.74 Å
   α=90 degrees
   β=109.5 degrees
   γ=90 degrees
Space group: P2₁/n
Molecules/unit cell: 8
Volume=706 Å³
   wherein measurement of said crystalline form is at a temperature of about −30° C.

In a different embodiment, the Form SA-1 of the hemi-H₂SO₄ salt of Compound I is characterized by the simulated powder x-ray diffraction pattern substantially in accordance with that shown in FIG. 34.A.

In still another embodiment, the Form SA-1 of the hemi-H₂SO₄ salt of Compound I is provided in substantially pure form.

The second crystalline form of a hemi-H₂SO₄ salt of Compound I comprises 0.5 mole of H₂SO₄ for each mole of Compound I, and further comprises water and dimethylformamide. This crystalline form is referred to herein as “Form SC-1” or “Form II.9”.

In one embodiment, Form SC-1 of the hemi-H₂SO₄ salt of Compound I is characterized by unit cell parameters substantially equal to the following:
Cell dimensions:
   a=16.94 Å
   b=16.82 Å
   c=20.99 Å
   α=90 degrees
   β=11.0 degrees
   γ=90 degrees
Space group: P2₁/n
Molecules/unit cell: 8
Volume=698 Å³
   wherein measurement of said crystalline form is at a temperature of about −100° C.

In a different embodiment, the Form SC-1 of the mono-H2SO₄ salt of Compound I is characterized by the simulated powder x-ray diffraction pattern substantially in accordance with that shown in FIG. 7.C and/or by the observed powder x-ray diffraction pattern substantially in accordance with that shown in FIG. 7.D.

In a different embodiment, the Form SC-1 of the hemi-H2SO₄ salt of Compound I is characterized by a powder x-ray diffraction pattern (CuKαλ=1.5418 Å) comprising four or more 2θ values, preferably comprising five or more 2θ values, selected from: 6.9±0.1, 7.8±0.1, 8.2±0.1, 10.4±0.1, 14.7±0.1, 22.7±0.1, 26.5±0.1, and 27.6±0.1, wherein measurement of the crystalline form is at a temperature of about 25° C.

In still another embodiment, the Form SD-2 of the mono-H₂SO₄ salt of Compound I is provided in substantially pure form.

Methanesulfonic Acid Salts of Compound I

Methanesulfonic acid (MSA) salts of Compound I include, for example, mono-MSA salts, which have a ratio of one mole of MSA to one mole of Compound I. The MSA salts of Compound I may optionally comprise one or more solvents, such as, for example, ethyl acetate, n-propanol, n-butanol, methyl isobutyl ketone, 1,2-dimethoxyethane, and propylene glycol.

In one embodiment, the MSA salt of Compound I is provided as a mono-MSA salt. Preferably, the mono-MSA salt is substantially pure. Further, the mono-MSA salt may be provided as crystalline material. An example of a crystalline form of the mono-MSA salt of Compound I includes a crystalline form comprising Form PG-1.

A crystalline form of a mono-MSA salt of Compound I comprises one mole of MSA for each mole of Compound I, and further comprises up to about one mole of propylene glycol for each molecule of Compound I. The crystalline form may optionally comprise water. This crystalline form is referred to herein as “Form PG-1” or “Form III.7”.

In one embodiment, Form PG-1 of the mono-MSA salt of Compound I is characterized by unit cell parameters substantially equal to the following:
Cell dimensions:
   a=22.50 Å
   b=8.55 Å
   c=17.49 Å
   α=90 degrees
   β=110.7 degrees
   γ=90 degrees
Space group: P2₁/a
Molecules/unit cell: 4
V/Z=787 Å³
Density (calculated)=1.325 g/cm³
   wherein measurement of said crystalline form is at a temperature of about −50° C.

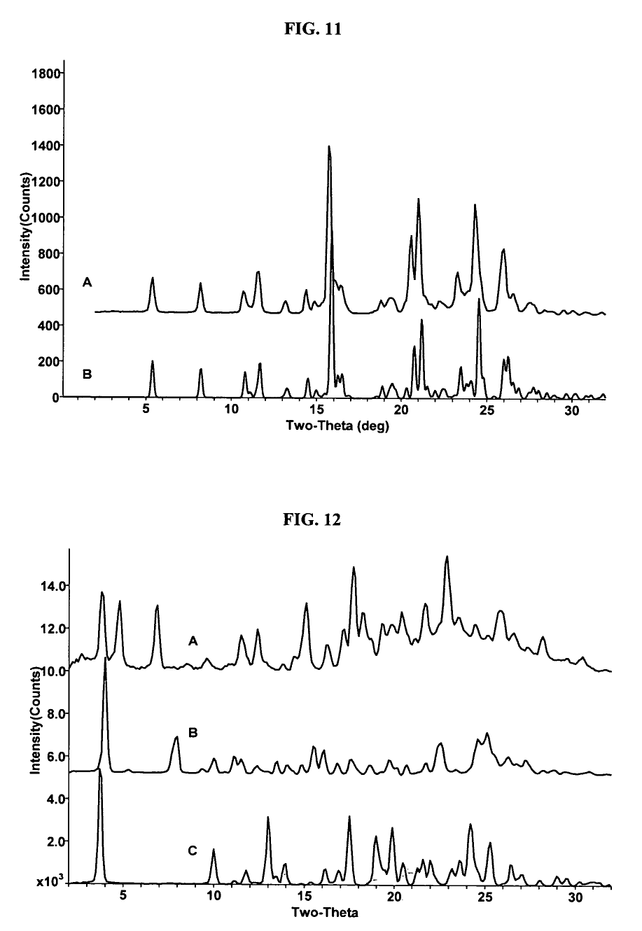

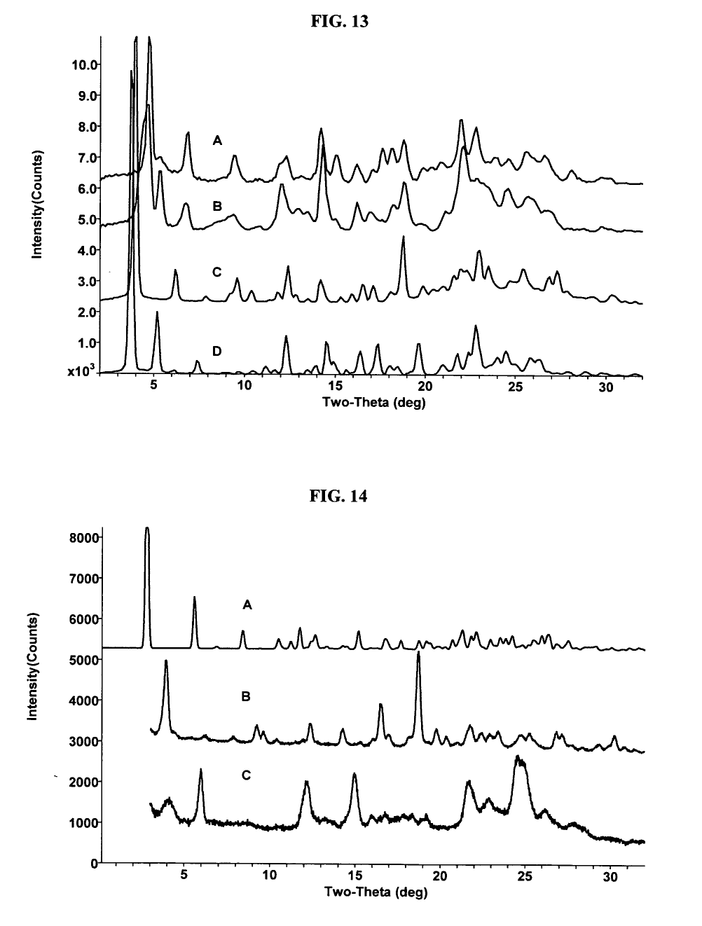

In a different embodiment, the Form PG-1 of the mono-MSA salt of Compound I is characterized by the observed powder x-ray diffraction pattern substantially in accordance with that shown in FIG. 11.A and/or the simulated powder x-ray diffraction pattern substantially in accordance with that shown in FIG. 11.B.

In a different embodiment, the Form PG-1 of the mono-MSA salt of Compound I is characterized by a powder x-ray diffraction pattern (CuKαλ=1.5418 Å) comprising four or more 2θ values, preferably comprising five or more 2θ values, selected from: 5.4±0.1, 8.2±0.1, 10.7±0.1, 11.6±0.1, 15.7±0.1, 20.6±0.1, 21.0±0.1, 23.3±0.1, and 24.4±0.1, wherein measurement of the crystalline form is at a temperature of about 25° C.

In still another embodiment, the Form PG-1 of the mono-MSA salt of Compound I is provided in substantially pure form.

In still another embodiment, the Form PG-1 of the mono-MSA salt of Compound I is provided in substantially pure form. This Form PG-1 of the mono-MSA salt of Compound I in substantially pure form may be employed in pharmaceutical compositions, which may optionally comprise one or more other components selected, for example, from excipients and carriers; and optionally, one or more other active pharmaceutical ingredients having active chemical entities of different molecular structures.

Preferably, the Form PG-1 of the mono-MSA salt of Compound I has substantially pure phase homogeneity as indicated by less than 10%, preferably less than 5%, and more preferably less than 2% of the total peak area in the experimentally measured powder x-ray diffraction (PXRD) pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from-the simulated PXRD pattern.

For example, the Form PG-1 of the mono-MSA salt of Compound I may be provided in substantially pure form, wherein substantially pure is greater than 90 weight % pure, preferably greater than 95 weight % pure, and more preferably greater than 99 weight % pure.

In a different embodiment, a composition is provided consisting essentially of Form PG-1 of the mono-MSA salt of Compound I. The composition of this embodiment may comprise at least 90 weight %, preferably at least 95 weight %, and more preferably at least 99 weight % of the Form PG-1 of the mono-MSA salt of Compound I, based on the weight of the mono-MSA salt of Compound I in the composition.

Phosphoric Acid Salts of Compound I

Phosphoric acid (H₃PO₄) salts of Compound I include, for example, a tri-H₃PO₄ salt, which has a ratio of three moles of H₃PO₄ to one mole of Compound I, and H₃PO₄ salts which have ratios of less than one mole of H₃PO₄ for each mole of Compound I. The H₃PO₄ salts of Compound I may optionally comprise water and/or one or more solvents, such as, for example, ethanol, acetic acid, and 1-methyl-2-pyrrolidinone (NMP).

In one embodiment, the H₃PO₄ salt of Compound I is provided as a tri-H₃PO₄ salt. Preferably, the tri-H₃PO₄ salt is substantially pure. Further, the tri-H₃PO₄ salt may be provided as crystalline material. An example of a crystalline form of the tri-H₃PO₄ salt of Compound I includes a crystalline form comprising Form SA-1.

A first crystalline form of a tri-H₃PO₄ salt of Compound I comprises three moles of H₂SO₄ for each mole of Compound I, and further comprises up to about one mole of water. This crystalline form may further comprise solvent, such as up to 0.5 mole N,N-dimethylacetamide for each mole of Compound I. This crystalline form is referred to herein as “Form SA-1” or “Form IV.10.

In one embodiment, Form SA-1 of the tri-H₃PO₄ salt of Compound I is characterized by unit cell parameters substantially equal to the following:
Cell dimensions:
   a=8.59 Å
   b=63.09 Å
   c=−14.05 Å
   α=90 degrees
   β=93.19 degrees
   γ=90 degrees
Space group: P2₁/n
Molecules/unit cell: 4
V/Z=1901 Å³
Density (calculated)=1.559 g/cm³
   wherein measurement of said crystalline form is at a temperature of about −60° C.

In a different embodiment, the Form SA-1 of the tri-H₃PO₄ salt of Compound I is characterized by the simulated powder x-ray diffraction pattern substantially in accordance with that shown in FIG. 14.A.

In a different embodiment, the Form SA-1 of the tri-H₃PO₄ salt of Compound I is characterized by a powder x-ray diffraction pattern (CuKαλ=1.5418 Å) comprising four or more 2θ values, preferably comprising five or more 2θ values, selected from: 2.8±0.1, 5.6±0.1, 8.4±0.1, 11.7±0.1, 15.2±0.1, 17.7±0.1, 21.3±0.1, 23.5±0.1, and 24.3±0.1, wherein measurement of the crystalline form is at a temperature of about 25° C.

In still another embodiment, the Form SA-1 of the tri-H₃PO₄ salt of Compound I is provided in substantially pure form.

Fumaric Acid Salts of Compound I

Fumaric acid salts of Compound I include, for example, hemi-fumaric acid salts, which have a ratio of 0.5 mole of fumaric acid to one mole of Compound I, and mono-fumaric acid salts which have ratios of one mole of fumaric acid for each mole of Compound I. The fumaric salts of Compound I may optionally comprise one or more solvents, such as, for example, ethanol, n-propanol, butyl acetate, acetone, methyl isobutyl ketone, heptane, and toluene.

In one embodiment, the fumaric acid salt of Compound I is provided as a hemi-fumaric acid salt. Preferably, the hemi-fumaric acid salt is substantially pure. Further, the hemi-fumaric acid salt may be provided as crystalline material. An example of a crystalline form of the hemi-fumaric acid salt of Compound I includes a crystalline form comprising Form TO-1.

One crystalline form of a hemi-fumaric acid salt of Compound I comprises 0.5 mole of fumaric acid for each mole of Compound I, and further comprises up to about one mole of toluene for each mole of Compound I. This crystalline form is referred to herein as “Form TO-1” or “Form VII.6”.

In one embodiment, Form TO-1 of the hemi-fumaric acid salt of Compound I is characterized by unit cell parameters substantially equal to the following:
Cell dimensions:
   a=22.31 Å
   b=8.54 Å
   c=18.92 Å
   α=90 degrees
   β=114.0 degrees
   γ=90 degrees
Space group: P2₁/n
Molecules/unit cell: 4
V/Z=823 Å³
Density (calculated)=1.288 g/cm³
   wherein measurement of said crystalline form is at a temperature of about 25° C.

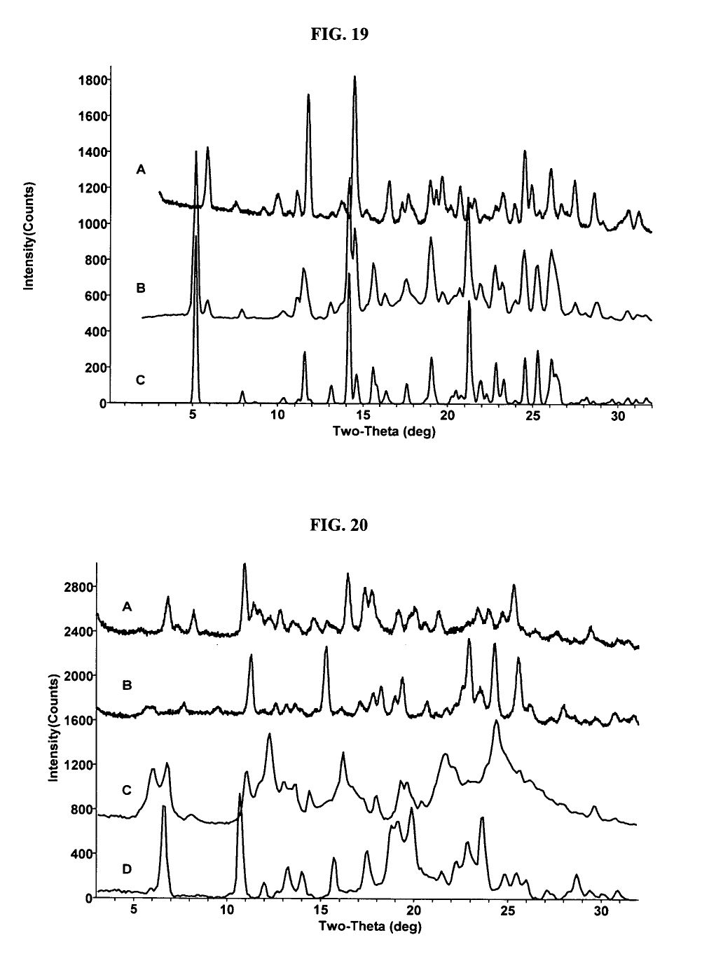

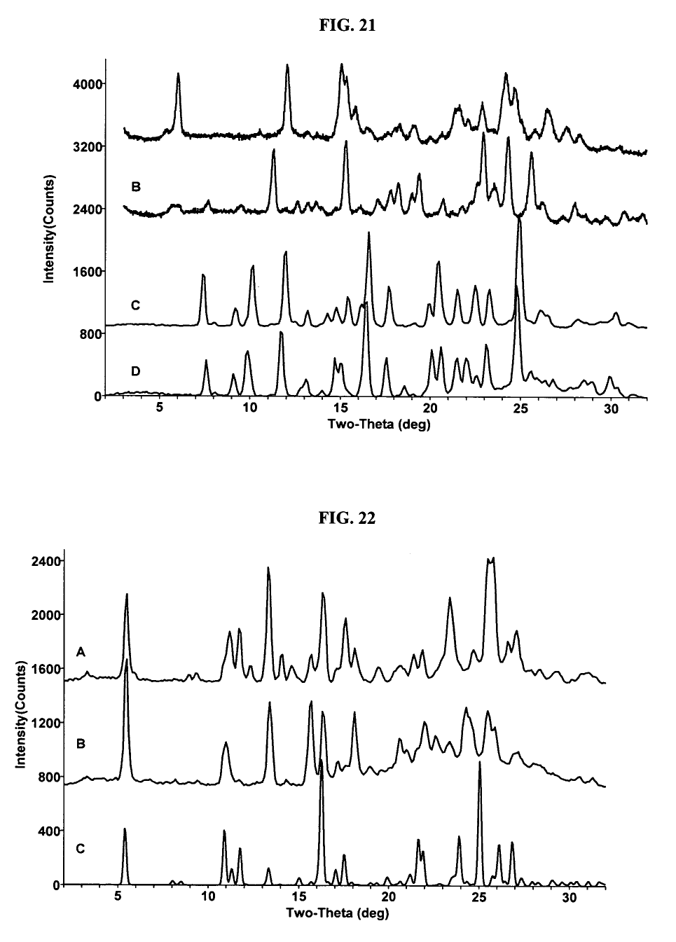

In a different embodiment, the Form TO-1 of the hemi-fumaric acid salt of Compound I is characterized by the observed powder x-ray diffraction pattern substantially in accordance with that shown in FIG. 19.C.

In a different embodiment, the Form TO-1 of the hemi-fumaric acid salt of Compound I is characterized by a powder x-ray diffraction pattern (CuKαλ=1.5418 Å) comprising four or more 2θ values, preferably comprising five or more 2θ values, selected from: 5.2±0.1, 11.6±0.1, 14.2±0.1, 15.6±0.1, 19.1±0.1, 21.3±0.1, 24.5±0.1, and 25.3±0.1, wherein measurement of the crystalline form is at a temperature of about25° C.

In still another embodiment, the Form TO-1 of the hemi-fumaric acid salt of Compound I is provided in substantially pure form.

Hydrobromic Acid Salt of Compound I

Hydrobromic acid (HBr) salts of Compound I include, for example, a mono-HBr salt which has a ratio of one mole of HBr for each mole of Compound I. The HBr salts of Compound I may optionally comprise water and/or one or more solvents.

In one embodiment, the HBr salt of Compound I is provided as a mono-HBr salt. Preferably, the mono-HBr salt is substantially pure. Further, the mono-HBr salt may be provided as crystalline material. An example of a crystalline form of the mono-HBr salt of Compound I includes a crystalline form comprising Form H1.5-1.

One crystalline form of a mono-HBr salt of Compound I comprises one mole of HBr for each mole of Compound I, and further comprises up to about 1.5 moles of water for each molecule of Compound I. This crystalline form is referred to herein as “Form H1.5-1” or “Form X.1”.

In one embodiment, Form H1.5-1 of the mono-HBr salt of Compound I is characterized by unit cell parameters substantially equal to the following:
Cell dimensions:
   a=7.70 Å
   b=9.93 Å
   c=35.23 Å
   α=97.21 degrees
   β=94.56 degrees
   γ=91.98 degrees
Space group: Pbar1
Molecules/unit cell: 4
V/Z=665 Å³
Density (calculated)=1.510 g/cm³
   wherein measurement of said crystalline form is at a temperature of about −50° C.

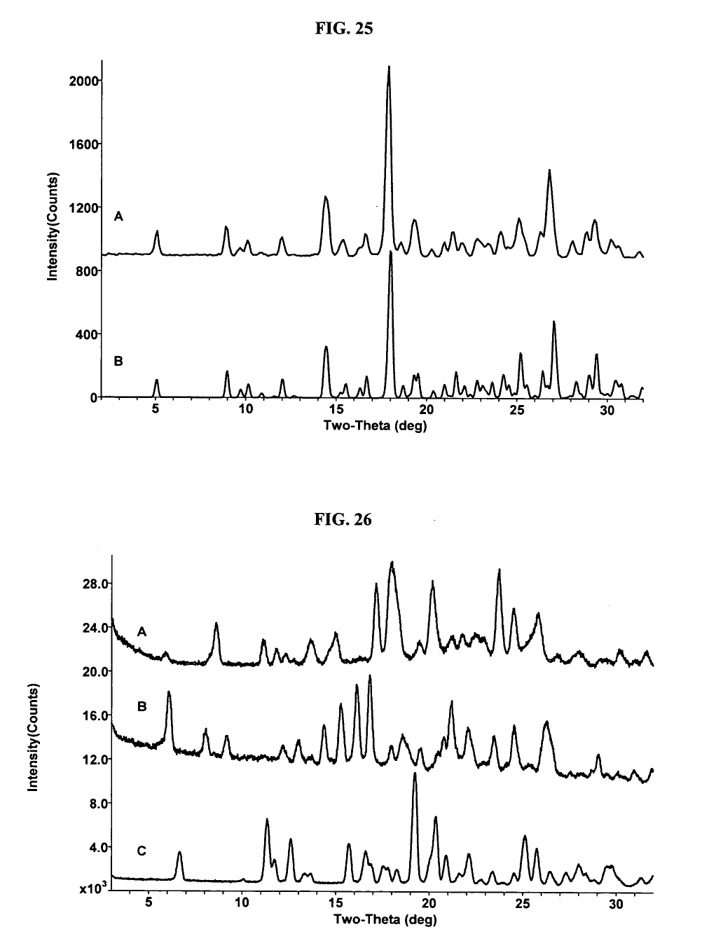

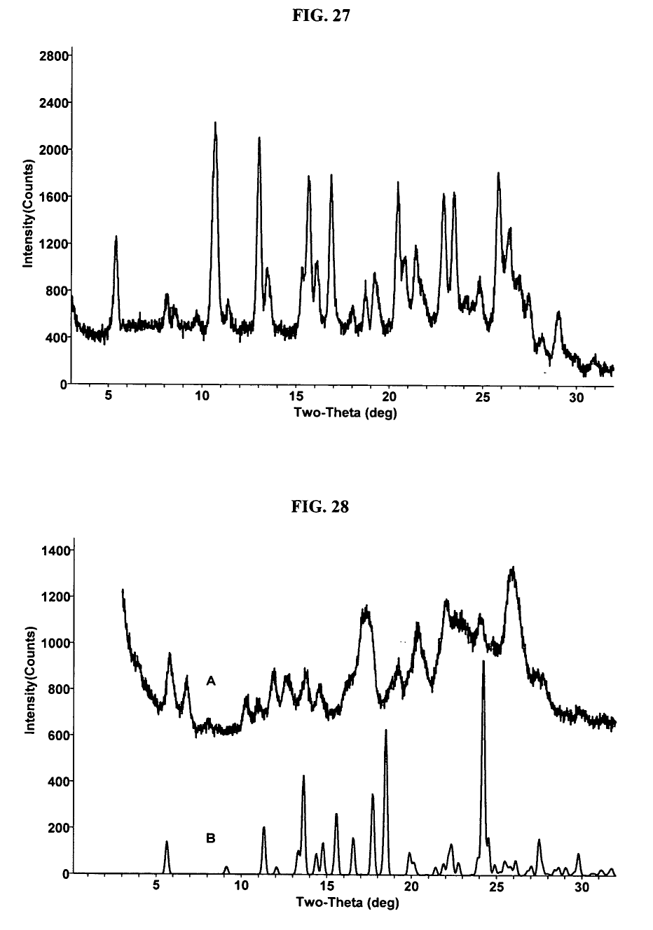

In a different embodiment, the Form H1.5-1 of the mono-HBr salt of Compound I is characterized by the simulated powder x-ray diffraction pattern substantially in accordance with that shown in FIG. 25.B and/or by the observed powder x-ray diffraction pattern substantially in accordance with that shown in FIG. 25.A.

In a different embodiment, the Form H1.5-1 of the mono-HBr salt of Compound I is characterized by a powder x-ray diffraction pattern (CuKαλ=1.5418 Å) comprising four or more 2θ values, preferably comprising five or more 2θ values, selected from: 5.0±0.1, 8.9±0.1, 14.4±0.1, 17.9±0.1, 24.1±0.1, 25.1±0.1, 26.9±0.1, 28.9±0.1, and 29.3±0.1, wherein measurement of the crystalline form is at a temperature of about 25° C.

In still another embodiment, the Form H1.5-1 of the mono-HBr salt of Compound I is provided in substantially pure form.

In still another embodiment, the Form H1.5-1 of the mono-HBr salt of Compound I is provided in substantially pure form. This Form H1.5-1 of the mono-HBr salt of Compound I in substantially pure form may be employed in pharmaceutical compositions, which may optionally comprise one or more other components selected, for example, from excipients and carriers; and optionally, one or more other active pharmaceutical ingredients having active chemical entities of different molecular structures.

Preferably, the Form H1.5-1 of the mono-HBr salt of Compound I has substantially pure phase homogeneity as indicated by less than 10%, preferably less than 5%, and more preferably less than 2% of the total peak area in the experimentally measured powder x-ray diffraction (PXRD) pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

For example, the Form H1.5-1 of the mono-HBr salt of Compound I may be provided in substantially pure form, wherein substantially pure is greater than 90 weight % pure, preferably greater than 95 weight % pure, and more preferably greater than 99 weight % pure.

In a different embodiment, a composition is provided consisting essentially of Form H1.5-1 of the mono-HBr salt of Compound I. The composition of this embodiment may comprise at least 90 weight %, preferably at least 95 weight %, and more preferably at least 99 weight % of the Form H1.5-1 of the mono-HBr salt of Compound I, based on the weight of the mono-HBr salt of Compound I in the composition.

Acetic Acid Salts of Compound I

Acetic acid salts of Compound I include, for example, mono-acetic acid salts which have a ratio of one mole of acetic acid for each mole of Compound I. The acetic acid salts of Compound I may optionally comprise one or more solvents, such as, for example, 1-methyl-2-pyrrolidinone and methyl isobutyl ketone.

In one embodiment, the acetic acid salt of Compound I is provided as a mono-acetic acid salt. Preferably, the mono-acetic acid salt is substantially pure. Further, the mono-acetic acid salt may be provided as crystalline material. An example of a crystalline form of the mono-acetic acid salt of Compound I includes a crystalline form comprising Form NMP-1.

One crystalline form of a mono-acetic acid salt of Compound I comprises one mole of acetic acid for each mole of Compound I, and further comprises up to about one mole of 1-methyl-2-pyrrolidinone for each mole of Compound I. This crystalline form is referred to herein as “Form NMP-1” or “Form XIII.2”.

In one embodiment, Form NMP-1 of the mono-acetic acid salt of Compound I is characterized by unit cell parameters substantially equal to the following:
Cell dimensions:
   a=8.37 Å
   b=15.62 Å
   c=14.02 Å
   α=90 degrees
   β=118.6 degrees
   γ=90 degrees
Space group: Pc
Molecules/unit cell: 2
Volume=805 Å³
Density (calculated)=1.335 g/cm³
   wherein measurement of said crystalline form is at a temperature of about 25° C.

In a different embodiment, the Form NMP-1 of the mono-acetic acid salt of Compound I is characterized by the simulated powder x-ray diffraction pattern substantially in accordance with that shown in FIG. 28.B.

In a different embodiment, the Form NMP-1 of the mono-acetic acid salt of Compound I is characterized by a powder x-ray diffraction pattern (CuKαλ=1.5418 Å) comprising four or more 2θ values, preferably comprising five or more 2θ values, selected from: 5.7±0.1, 11.3±0.1, 13.6±0.1, 15.6±0.1, 16.5±0.1, 17.7±0.1, 18.5±0.1, and 24.2±0.1, wherein measurement of the crystalline form is at a temperature of about 25° C.

In still another embodiment, the Form NMP-1 of the mono-acetic acid salt of Compound I is provided in substantially pure form.

Salicylic Acid Salt of Compound I

Salicylic acid salts of Compound I include, for example, a mono-salicylic acid salt which has a ratio of one mole of salicylic acid for each mole of Compound I.

In one embodiment, the salicylic acid salt of Compound I is provided as a mono-salicylic acid salt. Preferably, the mono-salicylic acid salt is substantially pure. Further, the mono-salicylic acid salt may be provided as crystalline material. An example of a crystalline form of the mono-salicylic acid salt of Compound I includes a crystalline form comprising Form SS-2.

One crystalline form of a mono-salicylic acid salt of Compound I comprises one mole of salicylic acid for each mole of Compound I. This crystalline form may be prepared as a neat crystalline form, and is referred to herein as “Form SS-2” or “Form XVI.1”.

In one embodiment, Form SS-2 of the mono-salicylic acid salt of Compound I is characterized by unit cell parameters substantially equal to the following:
Cell dimensions:
   a=22.24 Å
   b=8.94 Å
   c=14.87 Å
   α=90 degrees
   β=94.1 degrees
   γ=90 degrees
Space group: P2₁/a
Molecules/unit cell: 4
V/Z=737 Å³
Density (calculated)=1.411 g/cm³
   wherein measurement of said crystalline form is at a temperature of about −40° C.

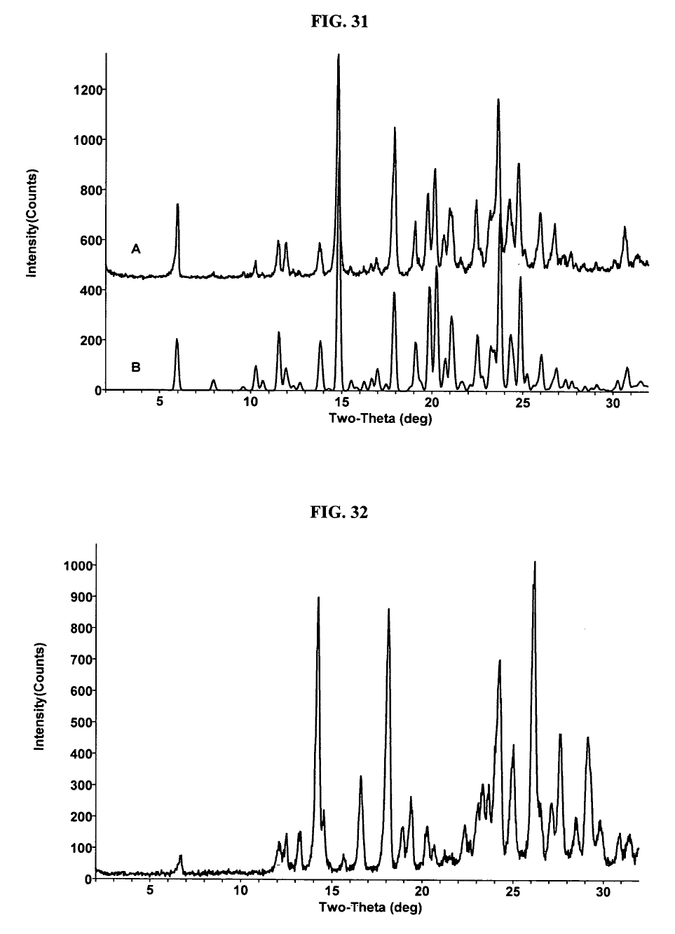

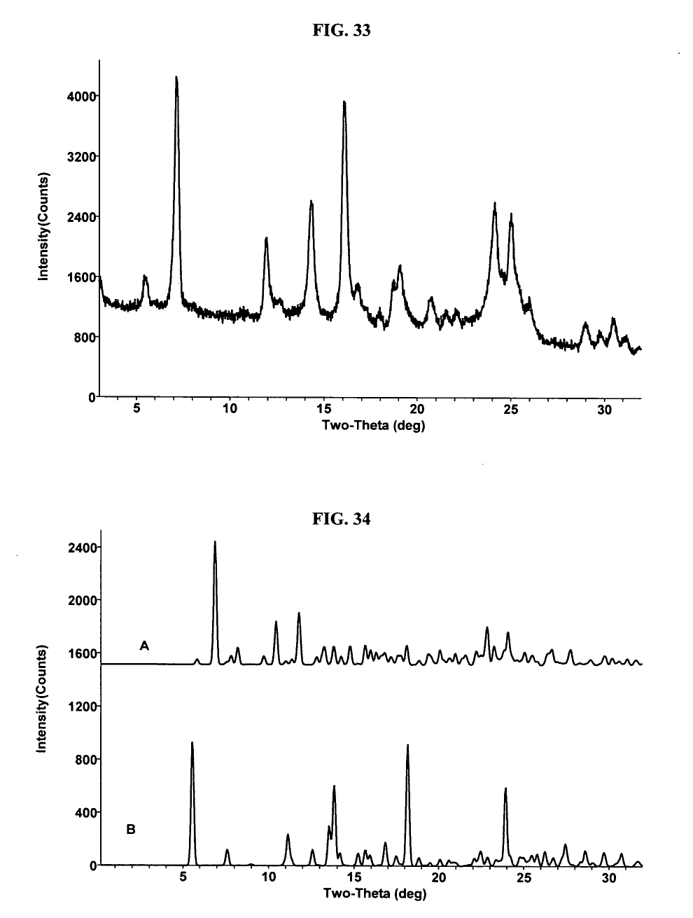

In a different embodiment, the Form SS-2 of the mono-salicylic acid salt of Compound I is characterized by the simulated powder x-ray diffraction pattern substantially in accordance with that shown in FIG. 31.B and/or by the observed powder x-ray diffraction pattern substantially in accordance with that shown in FIG. 31.A.

In a different embodiment, the Form SS-2 of the mono-salicylic acid salt of Compound I is characterized by a powder x-ray diffraction pattern (CuKαλ=1.5418 Å) comprising four or more 2θ values, preferably comprising five or more 2θ values, selected from: 5.9±0.1, 13.8±0.1, 14.8±0.1, 17.9±0.1, 19.8±0.1, 20.2±0.1, 23.7±0.1, and 24.8±0.1, wherein measurement of the crystalline form is at a temperature of about 25° C.

In still another embodiment, the Form SS-2 of the mono-salicylic acid salt of Compound I is provided in substantially pure form.

In still another embodiment, the Form SS-2 of the mono-salicylic acid salt of Compound I is provided in substantially pure form. This Form SS-2 of the mono-salicylic acid salt of Compound I in substantially pure form may be employed in pharmaceutical compositions, which may optionally comprise one or more other components selected, for example, from excipients and carriers; and optionally, one or more other active pharmaceutical ingredients having active chemical entities of different molecular structures.

Preferably, the Form SS-2 of the mono-salicylic acid salt of Compound I has substantially pure phase homogeneity as indicated by less than 10%, preferably less than 5%, and more preferably less than 2% of the total peak area in the experimentally measured powder x-ray diffraction (PXRD) pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

For example, the Form SS-2 of the mono-salicylic acid salt of Compound I may be provided in substantially pure form, wherein substantially pure is greater than 90 weight % pure, preferably greater than 95 weight % pure, and more preferably greater than 99 weight % pure.

In a different embodiment, a composition is provided consisting essentially of Form SS-2 of the mono-salicylic acid salt of Compound I. The composition of this embodiment may comprise at least 90 weight %, preferably at least 95 weight %, and more preferably at least 99 weight % of the Form SS-2 of the mono-salicylic acid salt of Compound I, based on the weight of the mono-salicylic acid salt of Compound I in the composition.

Tartaric Acid Salt of Compound I

Tartaric acid salts of Compound I include, for example, a mono-tartaric acid salt which has a ratio of one mole of tartaric acid for each mole of Compound I. The tartaric acid salts may be prepared from either D-tartaric acid, L-tartaric acid, or from mixtures of D- and L- tartaric acid, such as a racemic mixture.

In one embodiment, the tartaric acid salt of Compound I is provided as a mono-tartaric acid salt. The mono-tartaric acid salt is prepared from either D-tartaric acid or from L-tartaric acid. Preferably, the mono-tartaric acid salt is substantially pure. Further, the tartaric acid salt may be provided as crystalline material.

A tartaric acid salt of Compound I may be provided as crystals that comprise one mole of D-tartaric acid for each molecule of Compound I or one mole of L-tartaric acid for each molecule of Compound I. These crystals are enantiomorphs and are referred to herein as “Form V.1” and “Form V.2”, which were prepared from D-tartaric acid and L-tartaric acid, respectively, in Example 5.1. Form V.1 and/or Form V.2 may be prepared as a neat crystalline forms.

In one embodiment, the crystalline Form V.1 and/or Form V.2 of the tartaric acid salt of Compound I are characterized by unit cell parameters substantially equal to the following:
Cell dimensions:
   a=5.68 Å
   b=11.94 Å
   c=24.62 Å
   α=90 degrees
   β=91.7 degrees
   γ=90 degrees
Space group: P2₁
Molecules/unit cell: 2
V/Z=834 Å³
Density (calculated)=1.278 g/cm³
   wherein measurement of said crystalline form is at a temperature of about −50° C.

In a different embodiment, Form V.1 of the tartaric acid salt of Compound I is characterized by the simulated powder x-ray diffraction pattern substantially in accordance with that shown in FIG. 16.B and/or by the observed powder x-ray diffraction pattern substantially in accordance with that shown in FIG. 16.A.

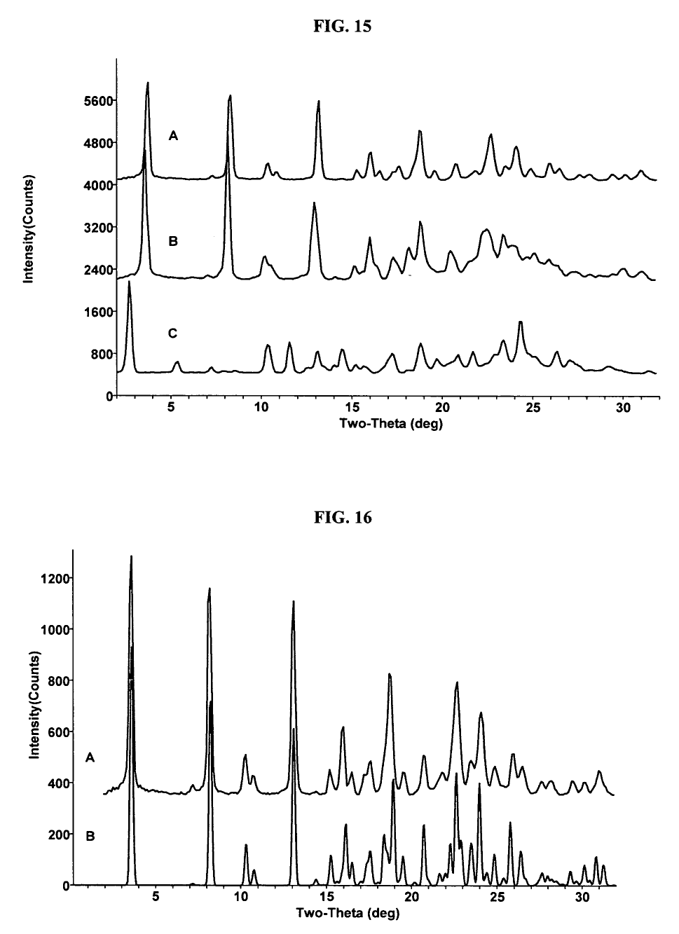

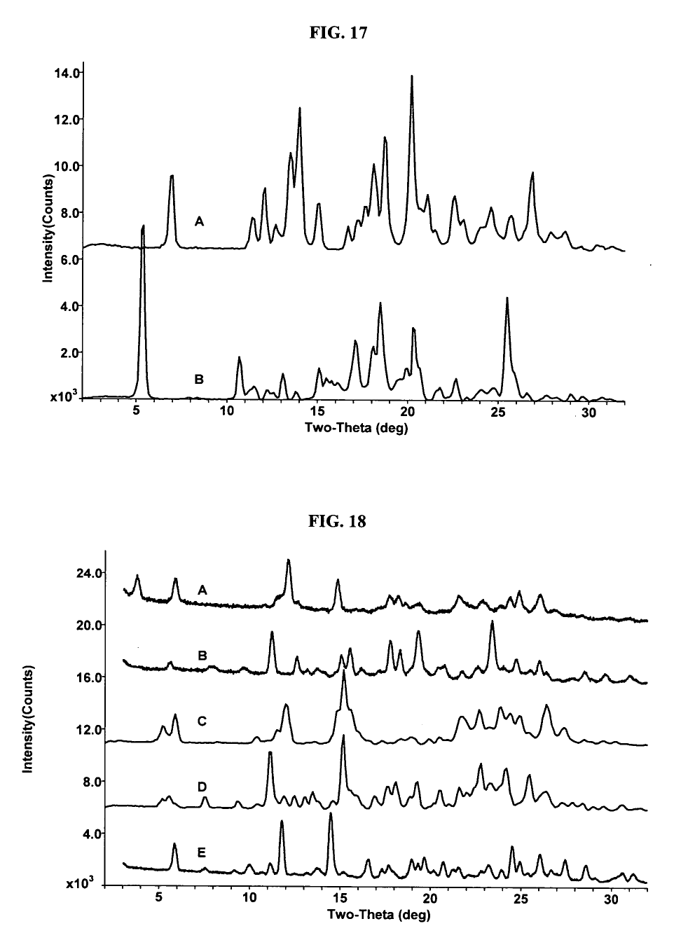

In a different embodiment, Form V.2 of the tartaric acid salt of Compound I is characterized by the simulated powder x-ray diffraction pattern substantially in accordance with that shown in FIG. 16.B and/or by the observed powder x-ray diffraction pattern substantially in accordance with that shown in FIG. 15.B.

In still another embodiment, Form V.1 and/or Form V.2 of the tartaric acid salt of Compound I is provided in substantially pure form.

In still another embodiment, Form V.1 and/or Form V.2 of the mono-tartaric acid salt of Compound I is provided in substantially pure form. These forms of the mono-tartaric acid salt of Compound I in substantially pure form may be employed in pharmaceutical compositions, which may optionally comprise one or more other components selected, for example, from excipients and carriers; and optionally, one or more other active pharmaceutical ingredients having active chemical entities of different molecular structures.

Preferably, Form V.1 and/or Form V.2 of the mono-tartaric acid salt of Compound I has substantially pure phase homogeneity as indicated by less than 10%, preferably less than 5%, and more preferably less than 2% of the total peak area in the experimentally measured powder x-ray diffraction (PXRD) pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

For example, the Form V.1 and/or Form V.2 of the mono-tartaric acid salt of Compound I may be provided in substantially pure form, wherein substantially pure is greater than 90 weight % pure, preferably greater than 95 weight % pure, and more preferably greater than 99 weight % pure.

In a different embodiment, a composition is provided consisting essentially of Form V.1 of the D-tartaric acid salt of Compound I. The composition of this embodiment may comprise at least 90 weight %, preferably at least 95 weight %, and more preferably at least 99 weight % of the Form V.1 of the mono-tartaric acid salt of Compound I, based on the weight of the mono-tartaric acid salt of Compound I in the composition.

In another embodiment, a composition is provided consisting essentially of Form V.2 of the L-tartaric acid salt of Compound I. The composition of this embodiment may comprise at least 90 weight %, preferably at least 95 weight %, and more preferably at least 99 weight % of the Form V.2 of the mono-tartaric acid salt of Compound I, based on the weight of the mono-tartaric acid salt of Compound I in the composition.

P-Toluenesulfonic Acid Salt of Compound I

p-Toluenesulfonic acid salts of Compound I include, for example, a mono-p-toluenesulfonic acid salt which has a ratio of one mole of p-toluenesulfonic acid for each mole of Compound I.

In one embodiment, the p-toluenesulfonic acid salt of Compound I is provided as a mono-p-toluenesulfonic acid salt. Preferably, the mono-p-toluenesulfonic acid salt is substantially pure. Further, the mono-p-toluenesulfonic acid salt may be provided as crystalline material. An example of a crystalline form of the mono-p-toluenesulfonic acid salt of Compound I includes a crystalline form comprising Form N-1.

One crystalline form of a mono-p-toluenesulfonic acid salt of Compound I comprises one mole of p-toluenesulfonic acid for each mole of Compound I. This crystalline form may be prepared as a neat crystalline form, and is referred to herein as “Form N-1” or “Form XIV.2”.

In one embodiment, Form N-1 of the mono-p-toluenesulfonic acid salt of Compound I is characterized by unit cell parameters substantially equal to the following:
Cell dimensions:
   a=11.85 Å
   b=19.04 Å
   c=15.60 Å
   α=90 degrees
   β=116.6 degrees
   γ=90 degrees
Space group: P2₁/c
Molecules/unit cell: 4
V/Z=787 Å³
Density (calculated)=1.394 g/cm³
   wherein measurement of said crystalline form is at a temperature of about 25° C.

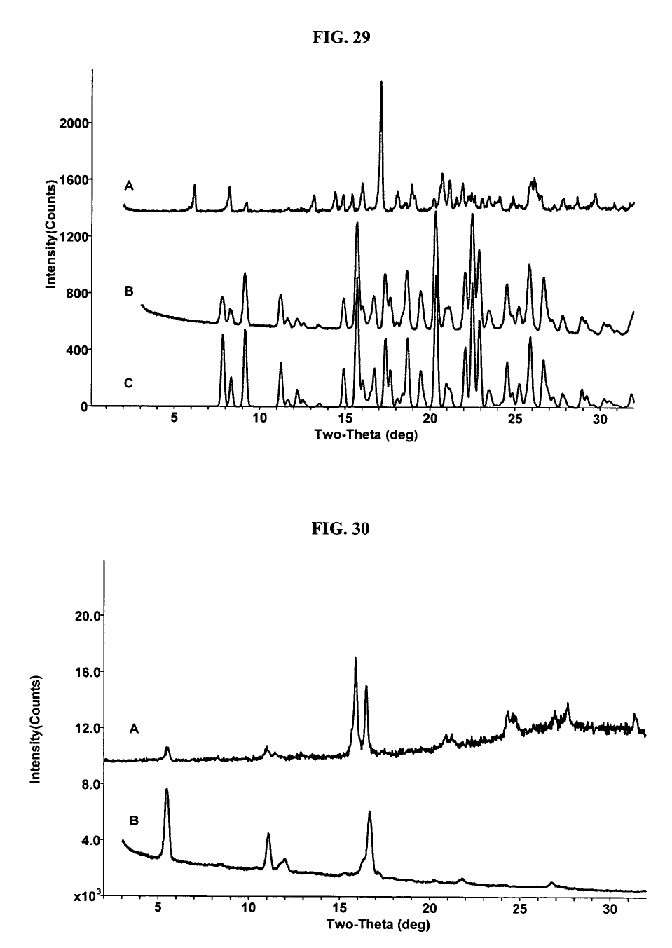

In a different embodiment, the Form N-1 of the mono-p-toluenesulfonic acid salt of Compound I is characterized by the simulated powder x-ray diffraction pattern substantially in accordance with that shown in FIG. 29.C and/or by the observed powder x-ray diffraction pattern substantially in accordance with that shown in FIG. 29.B.

In a different embodiment, the Form N-1 of the mono-p-toluenesulfonic acid salt of Compound I is characterized by a powder x-ray diffraction pattern (CuKαλ=1.5418 Å) comprising four or more 2θ values, preferably comprising five or more 2θ values, selected from:7.8±0.1, 8.3±0.1, 9.2±0.1, 15.7±0.1, 20.4±0.1, 22.1±0.1, 22.5±0.1, and 22.9±0.1, wherein measurement of the crystalline form is at a temperature of about 25° C.

In still another embodiment, the Form N-1 of the mono-p-toluenesulfonic acid salt of Compound I is provided in substantially pure form.

In still another embodiment, the Form N-1 of the mono-p-toluenesulfonic salt of Compound I is provided in substantially pure form. This Form N-1 of the mono-p-toluenesulfonic acid salt of Compound I in substantially pure form may be employed in pharmaceutical compositions, which may optionally comprise one or more other components selected, for example, from excipients and carriers; and optionally, one or more other active pharmaceutical ingredients having active chemical entities of different molecular structures.

Preferably, the Form N-1 of the mono-p-toluenesulfonic acid salt of Compound I has substantially pure phase homogeneity as indicated by less than 10%, preferably less than 5%, and more preferably less than 2% of the total peak area in the experimentally measured powder x-ray diffraction (PXRD) pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

For example, the Form N-1 of the mono-p-toluenesulfonic acid salt of Compound I may be provided in substantially pure form, wherein substantially pure is greater than 90 weight % pure, preferably greater than 95 weight % pure, and more preferably greater than 99 weight % pure.

In a different embodiment, a composition is provided consisting essentially of Form N-1 of the mono-p-toluenesulfonic acid salt of Compound I. The composition of this embodiment may comprise at least 90 weight %, preferably at least 95 weight %, and more preferably at least 99 weight % of the Form N-1 of the mono-p-toluenesulfonic acid salt of Compound I, based on the weight of the mono-p-toluenesulfonic acid salt of Compound I in the composition.

Maleic Acid Salt of Compound I

Maleic acid salts of Compound I include, for example, a mono-maleic acid salt which has a ratio of one mole of maleic acid and one mole of ethanol for each mole of Compound I; and a mono-maleic acid salt which has a ratio of one mole of maleic acid and up to three moles of water per mole of Compound I.

In one embodiment, the maleic acid salt of Compound I is provided as a mono-maleic acid salt. Preferably, the mono-maleic acid salt is substantially pure. Further, the mono-maleic acid salt may be provided as crystalline material. Examples of crystalline forms of the mono-maleic acid salt of Compound I include a first crystalline form comprising Form E-1 and a second crystalline form comprising Form H3-2.

The first crystalline form of a mono-maleic acid salt of Compound I comprises one mole of maleic acid and one mole of ethanol for each mole of Compound I. This crystalline form may be prepared as a neat crystalline form, and is referred to herein as “Form E-1” or “Form VIII.6”.

In one embodiment, Form E-1 of the mono-maleic acid salt of Compound I is characterized by unit cell parameters substantially equal to the following:
Cell dimensions:
   a=35.64 Å
   b=8.36 Å
   c=22.70 Å
   α=90 degrees
   β=113.58 degrees
   γ=90 degrees
Space group: C2/c
Molecules/unit cell: 8
V/Z=775 Å³
Density (calculated)=1.392 g/cm³
   wherein measurement of said crystalline form is at a temperature of about −50° C.

In a different embodiment, the Form E-1 of the mono-maleic acid salt of Compound I is characterized by the simulated powder x-ray diffraction pattern substantially in accordance with that shown in FIG. 22.C.

In a different embodiment, the Form E-1 of the mono-maleic acid salt of Compound I is characterized by a powder x-ray diffraction pattern (CuKαλ=1.5418 Å) comprising four or more 2θ values, preferably comprising five or more 2θ values, selected from: 5.4±0.1, 10.8±0.1, 11.2±0.1, 13.2±0.1, 16.2±0.1, 21.4±0.1, 21.7±0.1, 24.8±0.1, and 26.6±0.1, wherein measurement of the crystalline form is at a temperature of about 25° C.

In still another embodiment, the Form E-1 of the mono-maleic acid salt of Compound I is provided in substantially pure form.

The second crystalline form of a mono-maleic acid salt of Compound I comprises one mole of maleic acid and up to three moles of water for each mole of Compound I. This crystalline form may be prepared as a neat crystalline form, and is referred to herein as “Form H3-2” or “Form VIII.3”.

In one embodiment, Form H3-2 of the mono-maleic acid salt of Compound I is characterized by unit cell parameters substantially equal to the following:
Cell dimensions:
   a=17.06 Å
   b=7.78 Å
   c=23.10 Å
   α=90 degrees
   β=91.89 degrees
   γ=90 degrees
Space group: P2₁/c
Molecules/unit cell: 4
V/Z=767 Å³
Density (calculated)=1.425 g/cm³
   wherein measurement of said crystalline form is at a temperature of about −70° C.

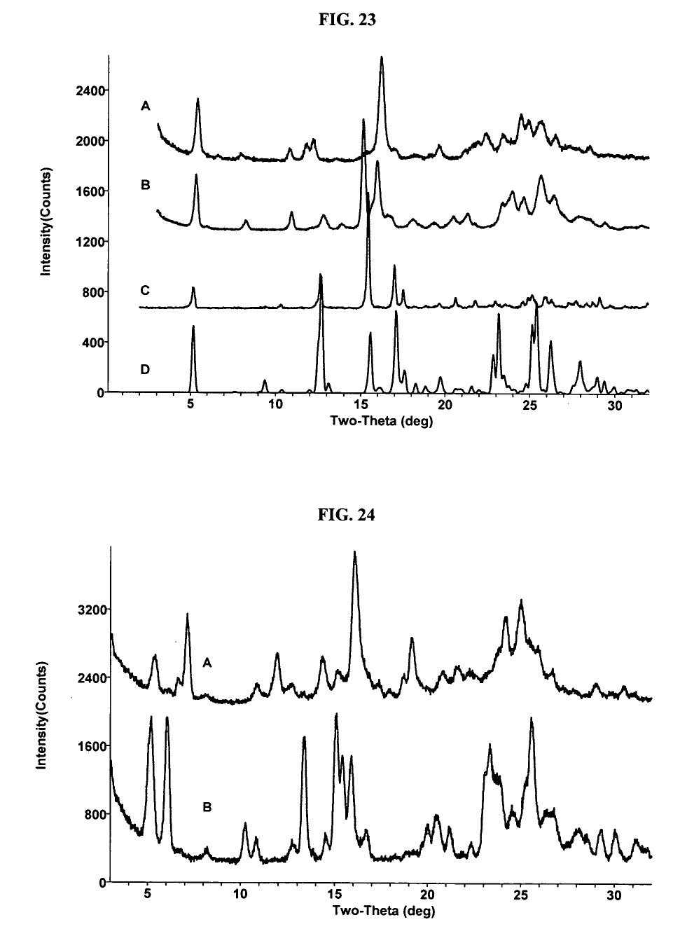

In a different embodiment, the Form H3-2 of the mono-maleic acid salt of Compound I is characterized by the simulated powder x-ray diffraction pattern substantially in accordance with that shown in FIG. 23.D and/or by the observed powder x-ray diffraction pattern substantially in accordance with that shown in FIG. 23.C.

In a different embodiment, the Form H3-2 of the mono-maleic acid salt of Compound I is characterized by a powder x-ray diffraction pattern (CuKαλ=1.5418 Å) comprising four or more 2θ values, preferably comprising five or more 2θ values, selected from: 5.1±0.1, 9.4±0.1, 12.6±0.1, 15.4±0.1, 22.6±0.1, 23.0±0.1, and 25.2±0.1, wherein measurement of the crystalline form is at a temperature of about 25° C.

In still another embodiment, the Form H3-2 of the mono-maleic acid salt of Compound I is provided in substantially pure form.

In one embodiment, the pharmaceutical composition comprises at least one crystalline form of the Compound I salt and at least one pharmaceutically-acceptable excipient. Examples of suitable Compound I salts include, hydrobromic acid salts, hydrochloric acid salts, maleic acid salts, methanesulfonic acid salts, phosphoric acid salts, salicylic acid salts, sulfuric acid salts, p-toluenesulfonic acid salts, and tartaric acid salts. Preferably, the pharmaceutical composition comprises at least one Compound I salt, wherein the Compound I salt is substantially pure.

In another embodiment, the pharmaceutical composition comprises at least one Compound I salt, wherein the at least one Compound I salt is in a crystalline form; and at least one pharmaceutically-acceptable excipient. Examples of suitable crystalline forms of Compound I salts include, but are not limited to, Form 1.5-1 of a hydrobromic acid salt of Compound I, Form SS-2 of a salicylic acid salt of Compound I, Form PG-1 of a methanesulfonic acid salt of Compound I, Form N-1 of a p-toluenesulfonic acid salt of Compound I, Form V.1 of the D-tartaric acid salt of Compound I, and Form V.2 of the L-tartaric acid salt of Compound I. In one alternative embodiment, the pharmaceutical composition comprises a crystalline form of a salt of Compound I, wherein the crystalline form is substantially pure. In another embodiment, the pharmaceutical composition comprises a salt of Compound I, wherein the salt of Compound I consists essentially of one crystalline form. Preferably, the one crystalline form is substantially pure.

In another embodiment, the pharmaceutical composition comprises a single salt of Compound I and at least one pharmaceutically-acceptable excipient. Preferably the salt of Compound I is substantially pure. In this embodiment, it is preferred that the salt of Compound I comprises one crystalline form. Preferably, the salt of Compound I consists essentially of one crystalline form. More preferably, the one crystalline form is substantially pure.

One aspect of the invention is related to a method for treating a proliferative disease, comprising orally administering to a mammalian species in need thereof, a therapeutically effective amount of Compound I salt. Examples of suitable Compound I salts include, hydrobromic acid salts, hydrochloric acid salts, maleic acid salts, methanesulfonic acid salts, phosphoric acid salts, salicylic acid salts, sulfuric acid salts, p-toluenesulfonic acid salts, and tartaric acid salts. Preferably, the pharmaceutical composition comprises at least one Compound I salt, wherein the Compound I salt is substantially pure. Preferably, the Compound I salt is provided in a crystalline form. Examples of suitable crystalline forms of Compound I salts for the pharmaceutical composition include, but are not limited to, Form 1.5-1 of a hydrobromic acid salt of Compound I, Form SS-2 of a salicylic acid salt of Compound I, Form PG-1 of a methanesulfonic acid salt of Compound I, Form N-1 of a p-toluenesulfonic acid salt of Compound I, Form V.1 of the D-tartaric acid salt of Compound I, and Form V.2 of the L-tartaric acid salt of Compound I.

### Use and Utility

Compound I is a potent inhibitor of several selected and related oncogenic protein tyrosine kinases (PTKs): viz. BCR-ABL, c-SRC, c-KIT, PDGF receptor and EPH receptor. Each of these protein kinases has been strongly linked to multiple forms of human malignancies. Thus, Compound I is useful for the treatment of a variety of cancers, including, but not limited to, the following:
carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma);
hematopoietic tumors of lymphoid lineage including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, and Burketts lymphoma;
hematopoietic tumors of myeloid lineage including acute and chronic myelogenous leukemias, myelodysplastic syndrome, myeloid leukemia, and promyelocytic leukemia;
tumors of the central and peripheral nervous system including astrocytoma, neuroblastoma, glioma, and schwannomas;
tumors of mesenchymal origin including fibrosarcoma, rhabdomyosarcoma, and osteosarcoma; and
other tumors including melanoma, xeroderma pigmentosum, keratoacanthoma, seminoma, thyroid follicular cancer, and teratocarcinoma.

The methods and the pharmaceutical compositions are useful for the treatment of cancers such as chronic myelogenous leukemia (CML), gastrointestinal stromal tumor (GIST), chronic lymphocytic leukemia (CLL), small cell lung cancer (SCLC), non-small cell lung cancer (NSCLC), ovarian cancer, melanoma, mastocytosis, germ cell tumors, multiple myeloma, Philadelphia chromosome positive acute lymphoblastic leukemia (Ph+ ALL), acute myelogenous leukemia (AML), acute lymphoblastic leukemia (ALL), pediatric sarcomas, breast cancer, colorectal cancer, pancreatic cancer, head and neck cancer, prostate cancer and others known to be associated with protein tyrosine kinases such as, for example, SRC, BCR-ABL and c-KIT. The method and the pharmaceutical composition also useful in the treatment of cancers that are sensitive to and resistant to chemotherapeutic agents that target BCR-ABL and c-KIT, such as, for example, GLEEVAC® (STI-571), SKI 606, AZD0530, AP23848 (ARIAD), and AMN-107.

The methods and the pharmaceutical compositions are useful for the treatment of refractory cancers. A refractory cancer is resistant or unresponsive to treatment. In one embodiment, the methods and pharmaceutical composition are useful for the treatment of cancers that are resistant or unresponsive to treatment with Gleevac and/or AMN-107 (Novartis)

The method of treatment encompasses dosing protocols such as once a day for 2 to 10 days, every 3 to 9 days, every 4 to 8 days and every 5 days. In one embodiment there is a period of 3 days to 5 weeks, 4 days to 4 weeks, 5 days to 3 weeks, and 1 week to 2 weeks, in between cycles where there is no treatment. In another embodiment, Compound I and/or the salt of Compound I is administered orally once a day for 3 days, with a period of 1 week to 3 weeks in between cycles where there is no treatment. In yet another embodiment, Compound I and/or the salt of Compound I is administered orally once a day for 5 days, with a period of 1 week to 3 weeks in between cycles where there is no treatment.

In one embodiment, the treatment cycle for administration of Compound I and/or the salt of Compound I, is once daily for 5 consecutive days and the period between treatment cycles is from 2 to 10 days, or one week. In one embodiment, Compound I and/or the salt of Compound I is administered once daily for 5 consecutive days, followed by 2 days when there is no treatment.

The Compound I and/or the salt of Compound I can also be administered orally once every 1 to 10 weeks, every 2 to 8 weeks, every 3 to 6 weeks, and every 3 weeks.

In another embodiment, Compound I and/or the salt of Compound I is orally administered daily with no days off.

A dosage comprising a therapeutically effective amount of Compound I comprises Compound I, one or more acid salts of Compound I, or a combination of Compound I and one or more acid salts of Compound I. The actual dosage employed may be varied depending upon the requirements of the patient and the severity of the condition being treated. Determination of the proper dosage for a particular situation is within the skill of the art. The effective amount of Compound I (and Compound I salt) may be determined by one of ordinary skill in the art, and includes exemplary dosage amounts for an adult human of from about 15 to about 300 mg of Compound I per day, alternatively from about 50 to about 300 mg of Compound I per day, alternatively from about 100 to about 200 mg of Compound I per day, or alternatively from about 20 to about 100 mg of Compound I per day, which may be administered in a single dose or in the form of individual divided doses, such as from 2, 3, or 4 times per day. Alternatively, Compound I may be administered in a dose of about 50 to about 150 mg twice a day, for example, it may be dosed at 50, 70, 90, 100, 110, 120, 130, 140, or 150 mg twice a day. Alternatively, Compound I may be administered in a dose of about 100 to about 250 once daily, for example it may be dosed at 50, 70, 100, 120, 140, 160, 180, 200, 220, or 240 one a day. In one embodiment, Compound I is administered at 70 mg twice a day. In one embodiment, Compound I may be administered either continuously or on an alternating schedule, such as 5 day on, 2 days off, or some other schedule as described above. It will be understood that the specific dose level and frequency of dosing for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders.

Treatment can be initiated with smaller dosages that are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

Compound I and/or one or more Compound I salts may be employed alone or in combination with other suitable therapeutic agents useful in the treatment of protein tyrosine kinase-associated disorders such as PTK inhibitors other than Compound I, anti-inflammatories, antiproliferatives, chemotherapeutic agents, immunosuppressants, anticancer agents, and cytotoxic agents. Exemplary other therapeutic agents useful for treatment in combination with Compound I are disclosed in U.S. Pat. No. 6,596,746 B1.

The pharmaceutical composition can be provided as a solid composition, such as, for example, a tablet (e.g., chewable tables), capsule, caplet, or powder; or as a liquid composition, such as, for example, a solution or dispersion. The solid composition can be constituted or reconstituted with a liquid to provide a liquid dosage form prior to oral administration. Such dosage forms may be prepared by methods of pharmacy known to those skilled in the art (See Remington: The Science and Practice of Pharmacy, 20th ed., A. R. Gennaro, editor; 2000, Lippinocott Williams & Wilkins, Baltimore, Md.).

The oral dosage forms may further comprise at least one excipient. The excipients can take a wide variety of forms depending on the form of preparation desired for administration. For example, excipients suitable for use in solid oral dosage forms (e.g., powder, tablets, capsules, and caplets) include, but are not limited to, diluents, granulating agents, lubricants, binders, pH modifying agents, disintegrating agents, glidants, and surface active agents. Examples of excipients suitable for use in oral liquid dosage forms include, but are not limited to, water, glycerols, oils, alcohols, flavoring agents, preservatives, pH modifying agents, and coloring agents.

Binders suitable for use in pharmaceutical composition include, but are not limited to, starches such as corn starch and potato starch, sugars, microcrystalline cellulose, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, and sodium carboxymethyl cellulose), polyvinylpyrrolidone, methyl cellulose, pre-gelatinized starch, hydroxypropyl cellulose, hydroxypropyl methylcellulose, microcrystalline cellulose, and mixtures thereof. For example, the pharmaceutical composition may comprise binder in the range of from about 1 to 50 weight %, preferably in the range of from about 1 to about 20 weight %, based on the weight of the pharmaceutical composition.

Examples of fillers suitable for use in the pharmaceutical composition include, but are not limited to, lactose, calcium phosphate, talc, calcium carbonate, (e.g. granules or powder), microcrystalline cellulose, powdered cellulose, dextrates, kaolin, mannitol, silicic acid, sorbitol, starch, pre-gelatinized starch, and mixtures thereof. The optional filler in pharmaceutical compositions is typically present in from about 1 to about 95 weight percent of the pharmaceutical composition. For example, the filler may be present in the range of from about 2 to about 95 weight %, preferably in the range of from 10 to about 85 weight %, based on the weight of the pharmaceutical composition.

Disintegrants may be used in the pharmaceutical compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form the pharmaceutical composition and solid dosage forms. The amount of disintegrant used varies based upon the type of formulation, and is readily discernible to those of ordinary skill in the art. Typically, pharmaceutical compositions and dosage forms comprise from about 0.5 to about 15 weight percent of disintegrant, preferably from about 1 to about 10 weight percent of disintegrant, and more preferably from about 1 to about 5 weight % of the disintegrant, based on the weight of the pharmaceutical composition or dosage form. Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato, or tapioca starch, other starches, pre-gelatinized starch, other algins, other celluloses, gums, and mixtures thereof.

Lubricants that can be used in pharmaceutical compositions include, but are not limited to, calcium stearate, magnesium stearate, mineral oil, light mineral oil, glycerin, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. If used at all, lubricants are typically used in an amount of less than about 2 weight percent of the pharmaceutical composition into which they are incorporated. For example, the pharmaceutical composition may comprise from about 0.1 to about 3 weight % of the lubricant, preferably form about 0.2 to about 2 weight % of the lubricant, based on the weight of the pharmaceutical composition.

The pharmaceutical composition may further comprise one or more compounds that reduce the rate by which an active ingredient will decompose. Such compounds, which are referred to herein as “stabilizers” include, but are not limited to, antioxidants such as ascorbic acid and salt buffers.

Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing on a suitable machine with the active ingredients in a free flowing form such as powder or granules, optionally mixed with an excipient. Molded tablets can be made by molding on a suitable machine with a mixture of the powdered compound moistened with an inert liquid diluent.

Solutions for oral administration represent another convenient oral dosage form, in which case a solvent is employed. Liquid oral dosage forms are prepared by combining the active ingredient in a suitable solvent to form a solution, suspension, syrup, or elixir of the active ingredient in the liquid.

The solutions, suspensions, syrups, and elixirs may optionally comprise other additives including, but not limited to, glycerin, sorbitol, propylene glycol, sugars, flavoring agents, and stabilizers.

It is desirable to find new salts of Compound I with improved characteristics compared with the compound. Such salts may be amorphous, crystalline, and/or mixtures thereof. It is also desirable to find salts with advantageous and improved characteristics in one or more of the following categories: (a) improved purity; (b) pharmaceutical properties (i.e. solubility, permeability, amenability to sustained release formulations, bioavailability); (c) improved manufacturability (including factors that improve the manufacturing costs or feasibility, such as ease of handling, ease of formulation); (d) stability (including both kinetic and/or thermodynamic stability of the bulk drug substance, stability of the formulated product); (e) improved pharmacological characteristics (such as forms with improved protein tyrosine kinase inhibitory activity).

It is also desirable to find new crystalline forms of Compound I salts with improved characteristics compared with the known crystalline forms of the compound. It is also desirable to find crystalline forms of Compound I salts with advantageous and improved characteristics in one or more of the following categories: (a) improved purity; (b) pharmaceutical properties (i.e. solubility, permeability, amenability to sustained release formulations, bioavailability); (c) improved manufacturability (including factors that improve the manufacturing costs or feasibility, such as ease of handling, ease of formulation); (d) stability (including both kinetic and/or thermodynamic stability of the bulk drug substance, stability of the formulated product); (e) improved pharmacological characteristics (such as forms with improved protein tyrosine kinase inhibitory activity).

### Methods of Preparation and Characterization

Crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. High throughput crystallization techniques may be employed to prepare crystalline forms including polymorphs and are discussed in Morissette, Sherry L.; Soukasene, Stephen; Levinson, Douglas; Cima, Michael J.; Almarsson, Orn. Proceedings of the National Academy of Sciences of the United States of America (2003), 100(5), 2180-2184.

Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Ind. (1999).

For crystallization techniques that employ solvent, the choice of solvent or solvents is typically dependent upon one or more factors, such as solubility of the compound, crystallization technique, and vapor pressure of the solvent. Combinations of solvents may be employed, for example, the compound may be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which may be heated to promote dissolution. The term “slurry”, as used herein, means a saturated solution of the compound, which may also contain an additional amount of the compound to afford a heterogeneous mixture of the compound and a solvent at a given temperature.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971,26, 369-377. In general, seeds of small size are needed to control effectively the growth of crystals in the batch. Seeds of small size may be generated by sieving, milling, or micronizing of large crystals, or by micro-crystallization of solutions. Care should be taken that milling or micronizing of crystals does not result in any change in crystallinity from the desired crystal form (i.e., change to amorphous or to another polymorph).

A cooled crystallization mixture may be filtered under vacuum, and the isolated solids may be washed with a suitable solvent, such as cold recrystallization solvent, and dried under a nitrogen purge to afford the desired crystalline form. The isolated solids may be analyzed by a suitable spectroscopic or analytical technique, such as solid state nuclear magnetic resonance, differential scanning calorimetry, powder x-ray diffraction, or the like, to assure formation of the preferred crystalline form of the product. The resulting crystalline form may be produced in an amount of greater than about 70 weight % isolated yield, preferably greater than 90 weight % isolated yield, based on the weight of the compound originally employed in the crystallization procedure. The product may be comilled or passed through a mesh screen to delump the product, if necessary.

Crystalline forms may be prepared directly from the reaction medium of the final process for preparing Compound I. This may be achieved, for example, by employing in the final process step a solvent or a mixture of solvents from which Compound I may be crystallized. Alternatively, crystalline forms may be obtained by distillation or solvent addition techniques. Suitable solvents for this purpose include, for example, the aforementioned nonpolar solvents and polar solvents, including protic polar solvents such as alcohols, and aprotic-polar solvents such as ketones.

The presence of more than one crystalline form and/or polymorph in a sample may be determined by techniques such as powder x-ray diffraction (PXRD) or solid state nuclear magnetic resonance spectroscopy. For example, the presence of extra peaks in the comparison of an experimentally measured PXRD pattern with a simulated PXRD pattern may indicate more than one crystalline form and/or polymorph in the sample. The simulated PXRD may be calculated from single crystal x-ray data. see Smith, D. K., “A FORTRAN Program for Calculating X-Ray Powder Diffraction Patterns,” Lawrence Radiation Laboratory, Livermore, Calif., UCRL-7196 (April 1963).

Crystalline forms of Compound I salts may be characterized using various techniques, the operation of which are well known to those of ordinary skill in the art. The crystalline forms of Compound I salts may be characterized and distinguished using single crystal x-ray diffraction performed under standardized operating conditions and temperatures, which is based on unit cell measurements of a single crystal of the form at a fixed analytical temperature. The approximate unit cell dimensions in Angstroms (Å), as well as the crystalline cell volume, space group, molecules per cell, and crystal density may be measured, for example at a sample temperature of 25° C. A detailed description of unit cells is provided in Stout & Jensen, X-Ray Structure Determination: A Practical Guide, Macmillan Co., New York (1968), Chapter 3, which is herein incorporated by reference.

Alternatively, the unique arrangement of atoms in spatial relation within the crystalline lattice may be characterized according to the observed fractional atomic coordinates. Another means of characterizing the crystalline structure is by powder x-ray diffraction analysis in which the diffraction profile is compared to a simulated profile representing pure powder material, preferably both run at the same analytical temperature, and measurements for the subject form characterized as a series of 2θ values (usually four or more).

Other means of characterizing the form may be used, such as solid state nuclear magnetic resonance (NMR), differential scanning calorimetry, thermography, and gross examination of the crystalline or amorphous morphology. These parameters may also be used in combination to characterize the subject form.

The crystalline forms were analyzed using one or more of the testing methods described below.

Single Crystal X-Ray Measurements

Data were collected on a Bruker-Nonius CAD4 serial diffractometer Bruker AXS, Inc. Madison, Wis.). Unit cell parameters were obtained through least-squares analysis of the experimental diffractometer settings of 25 high-angle reflections. Intensities were measured using Cu Kα radiation (λ=1.5418 Å) at a constant temperature with the θ-2θ variable scan technique and were corrected only for Lorentz-polarization factors. Background counts were collected at the extremes of the scan for half of the time of the scan. Alternately, single crystal data were collected on a Bruker-Nonius Kappa CCD 2000 system using Cu Kα radiation (λ=1.5418 Å) or a Bruker AXS APEX2 X-ray system. Indexing and processing of the measured intensity data were carried out with the HKL2000 software package (Otwinowski, Z. and Minor, W., in Macromolecular Crystallography, eds. Carter, W. C. Jr and Sweet, R. M., Academic Press, NY, 1997) in the Collect program suite (Collect: Data collection software, R. Hooft, Nonius B. V., 1998) or the APEX2 Software Package (APEX2 User Manual, 2005, Bruker AXS, Inc., Madison, Wis.) When indicated, crystals were cooled in the cold stream of an Oxford Cryosystems Cryostream Cooler (Oxford Cryosystems, Inc., Devens, Mass.) during data collection.

The structures were solved by direct methods and refined on the basis of observed reflections using either the SDP software package (SDP Structure Determination Package, Enraf-Nonius, Bohemia, N.Y.) with minor local modifications or the crystallographic package, maXus (maXus Solution and Refinement Software Suite: S. Mackay, C. J. Gilmore, C. Edwards, M. Tremayne, N. Stewart, and K. Shankland).

The derived atomic parameters (coordinates and temperature factors) were refined through full matrix least-squares. The function minimized in the refinements was Σ_{w}(|Fₒ|−|F_{c}|)². R is defined as Σ∥Fₒ|−|F_{c}∥//Σ|Fₒ| while R_{w}=[Σ_{w}(|Fₒ|−|F_{c}|)²/Σ_{w}|Fₒ|²]^{1/2} where w is an appropriate weighting function based on errors in the observed intensities. Difference maps were examined at all stages of refinement. Hydrogen atoms were introduced in idealized positions with isotropic temperature factors, but no hydrogen parameters were varied.

Simulated powder x-ray diffraction patterns were generated from the single crystal atomic parameters at the data collection temperature, unless noted otherwise.

(Yin. S.; Scaringe, R. P.; DiMarco, J.; Galella, M. and Gougoutas, J. Z., American Pharmaceutical Review, 2003, 6, 2, 80).

Powder X-Ray Diffraction Measurements—Method A

Powder X-ray Diffraction (PXRD) (PhilPro): About 10 mg of crystalline sample was placed into a High Throughput X-Ray Diffraction Filter sample holder (described in U.S. Pat. No. 6,968,037). The sample was transferred to a PanAnalytical Philips PW3040 unit (45 KV, 40 mA, Cu Ka) x-ray diffraction unit (PanAnalytical Philips, Natick, Mass.). Data was collected at room temperature in the 2 to 32 degrees 2θ range (continuous scanning mode, scanning rate 0.0255 degrees/sec., Accelerator Detector, sample spinner: ON)

Powder X-Ray Diffraction Measurements—Method B

X-ray powder diffraction data were obtained using a Bruker C2 GADDS. The radiation was Cu Kα (40 KV, 50 mA). The sample-detector distance was 15 cm. Powder samples were placed in sealed glass capillaries of 1 mm or less in diameter; the capillary was rotated during data collection. Data were collected for 3≦2θ≦35° with a sample exposure time of at least 2000 seconds. The resulting two-dimensional diffraction arcs were integrated to create a traditional 1-dimensional PXRD pattern with a step size of 0.02 degrees 2θ n the range of 3 to 35 degrees 2θ.

Powder X-Ray Diffraction Measurements—Method C

X-ray powder diffraction (PXRD) data were obtained using a Bruker GADDS (General Area Detector Diffraction System) manual chi platform goniometer. Powder samples were placed in thin walled glass capillaries of 1 mm or less in diameter; the capillary was rotated during data collection. The sample-detector distance was 17 cm. The radiation was Cu Kα (λ=1.5418 Å). Data were collected for 3≦2θ≦35° with a sample exposure time of at least 300 seconds.

DSC (Sealed Pan)

Differential scanning calorimetry (DSC) experiments were performed in a TA Instruments™ model Q1000 or 2920. The sample (about 2-6 mg) was weighed in a pinpricked hermetically sealed aluminum pan and accurately recorded to a hundredth of a milligram, and transferred to the DSC. The instrument was purged with nitrogen gas at 50 mL/min. Data were collected between room temperature and 350° C. at 10° C./min heating rate. The plot was made with the endothermic peaks pointing down.

TGA (Sealed Pan)

Thermal gravimetric analysis (TGA) experiments were performed in a TA Instruments™ model Q500 or 2950. The sample (about 10-30 mg) was placed in a pinpricked hermetically sealed aluminum pan on a platinum pan, both previously tared. The weight of the sample was measured accurately and recorded to a thousandth of a milligram by the instrument. The furnace was purged with nitrogen gas at 100 mL/min. Data were collected between room temperature and 350° C. at 10° C./min heating rate.

Proton NMR

Proton NMR (pNMR): A solution was prepared by mixing approximately 10 mg of crystal sample into 0.6 mL of either DMSO-d₆ or DMSO-d₆ with a small amount of D₂O. The pNMR spectra was collected on a Bruker DPX 300 NMR (Bruker Biospin Corp, Billerica, Mass.) equipped with a Bruker Quad Nuclear Probe tuned to observe ¹H, ¹³C, ¹⁹F and ³¹p and a B-ACS 60 sample changer.

Raman Microspectroscopy

Experiments were performed in a Thermo-Nicolet Almega instrument. Raman spectra of salt samples of Compound I were collected over the spectral region of 200 to 3700 cm⁻¹ with two 5 second exposures using a 633 nm laser at ambient temperature.

### EXAMPLES

The following examples are provided, without any intended limitation, to further illustrate the present invention.

### Abbreviations

BSA benzenesulfonic acid
BuOAc butyl acetate
n-BuOH n-butanol
BuOEtOH butoxyethanol
DCM dichloromethane
DMA dimethylacetamide
DME 1,2-dimethoxyethane
DMF dimethylformamide
DMSO-d6 deuterated dimethyl sulfoxide
D₂O deuterium oxide
DSC differential scanning calorimetry
DME dimethoxyethane
EA elemental analysis
eq. mole equivalent
EtOAc ethyl acetate
EtOH ethanol
GC gas chromatography
HOAc acetic acid
iPrOH isopropanol
KF Karl Fischer titration
MeCN acetonitrile
MeOH methanol
MIBK methyl isobutyl ketone
MSA methanesulfonic acid
nBuOAc n-butyl acetate
NMP 1-methyl-2-pyrrolidinone
PG propylene glycol
pNMR proton nuclear magnetic resonance
pTSA p-toluenesulfonic acid
PXRD powder x-ray diffraction
THF tetrahydrofuran

The following general procedures were employed to prepare crystalline forms:
1. High throughput crystallization was employed to screen solvent, anti-solvent, and other crystallization parameters with 1 mg samples in 96 well plates.
2. Certain crystal forms were scaled up to about 40 mg samples using similar crystallization parameters as employed to prepare the 1 mg samples. Seed crystals, which were prepared in high throughput crystallization experiments utilizing the same crystallization solvents, were used in some crystallizations.
3. Certain crystalline forms were scaled up further to provide larger quantities. Seed crystals, which were prepared in a smaller scale experiment utilizing the same crystallization solvents, were used in some crystallizations.

Crystals of the monohydrate crystalline form, the butanolate crystalline form, an NMP solvate and an acetone solvate of Compound I (free base) were used as a source of Compound I or seed crystals in the certain preparations of the salt forms of Compound I.

### Preparation of Crystals of Compound I (Free Base)

### A. Preparation of the Monohydrate Crystalline form of Compound I (Free Base)

Crystals of a monohydrate crystalline form of Compound I may be prepared by the general procedure described below:

A suspension is prepared by admixing 48 g of the Compound I and approximately 1056 mL (22 mL/g) of ethyl alcohol, followed by the addition of approximately 144 mL of water. Next, Compound I is dissolved by heating the suspension to approximately 75° C. The Compound I solution is passed through a preheated filter and into a receiver vessel. The dissolution reactor and transfer lines are rinsed with a mixture of 43 mL EtOH and 5 mL of water. The contents of the receiver vessel are heated to approximately 75-80° C. and maintained at this temperature range to achieve complete dissolution. Next, approximately 384 mL of water is added at a rate such that the batch temperature is maintained between 75-80° C. The contents of the receiver vessel are cooled to 70° C. and then maintained at 70° C. for about 1 hour. The temperature is decreased to 5° C. over a period of 2 hours, and maintained between 0-5° C. for at least 2 hours. The resulting crystal slurry is filtered. The filter cake is washed with a mixture of 96 mL EtOH and 96 mL of water. The crystals are dried at ≦50° C. under reduced pressure until the water content is in the range of from 3.4 to 4.1% by KF to afford 41 g (85 M %).

### B. Preparation of the Butanolate Crystalline Form of Compound I (Free Base)

Crystals of a butanolate crystalline form of Compound I may be prepared by the general procedure described below:

Compound I is dissolved into 1-butanol at reflux (116-118° C.) at a concentration of approximately 1 g/25 mL of solvent. Upon cooling, Compound I crystallizes out of solution as the butanol solvate. The resulting crystals are filtered, washed with butanol, and dried.

### C. Preparation of the NMP Partially Solvated Form of Compound I (Free Base)

2-(6-Chloro-2-methylpyrimidin-4-ylamino)-N-(2-chloro-6-methylphenyl)thiazole-5-carboxamide (7.8 g) is placed in a flask with 13 g of hydroxyethylpiperazine, 6.97 mL of diisopropylethylamine and 51 mL of NMP. The mixture is heated to 110° C. for 45 minutes and then cooled to ambient temperature. 360 mL of water is slowly added to afford a heavy slurry. The slurry is filtered, washed with 150 mL of water and dried to afford 9.17 g of Compound I containing residual NMP.

### D. Preparation of Acetone-water Solvate

A mixture is prepared by admixing 15 g of 2-(6-Chloro-2-methylpyrimidin-4-ylamino)-N-(2-chloro-6-methylphenyl)thiazole-5-carboxamide, 13.2 mL of DIPEA, 23.3 g of hydroxyethylpiperazine and 300 mL of dioxane. The mixture is heated to reflux for 12 hours and then allowed to cool to ambient temperature. Solvent is removed under vacuum and water is added to prepare a slurry. The resulting solids are isolated and washed with water. The solids are slurried into ethyl ether twice. Then the slurry is triturated two times with hot acetone to afford 16.76 g of Compound I containing 0.85 eq. of water and 1.1 eq. of acetone.

### General Procedure for High Throughput Crystallization

A master stock solution was prepared for each Compound I salt. For a 96 well plate, approximately 100 mg of Compound I in 8 mL of solvent was used to prepare the master stock solution. Master stock solution was dispensed into wells with a Gilson 215 eight probe liquid handler (Middleton, Wis.) or a multi-channel pipettor. The solvent was evaporated on a Savant Speed-Vac evaporator (Thermo Electron Corp., Waltham, Mass.) and test solvents and anti-solvents (100 μL total volume) were applied to the wells. The plates were sealed with septa to minimize evaporation of the test solvent anti-solvent combinations.

After the plates were incubated for the desired amount of time, the contents of the wells were photographed with a custom Wellplate Inspection System (Coleman Technologies). Wells containing birefringent solids were analyzed by Raman Microspectroscopy on an Almega system equipped with 633 nm and 785 nm lasers (Thermo-Nicolet, Waltham, Mass.). The wells were then sorted into groups by OMNIC software (Thermo-Nicolet).

Certain crystalline forms were scaled up to provide larger quantities. Master stock solutions of Compound I with counterion were prepared to provide approximately 40 mg of Compound I per sample. Samples were prepared by dividing the master stock solutions into smaller tubes and removing the solvent by evaporation with a Savant Speed Vac evaporator. The test solvents and anti-solvents were then added to the samples. The samples were heated and stirred for various periods of time. Crystalline samples from successful crystallizations were then isolated onto a High Throughput X-Ray Diffraction Filter sample holder (WO 2003087796 A1) and analyzed on a Philips PW3040 X-ray Diffraction unit (PanAnalytical Philips, Natick, Mass.).

### Example 1

### Hydrochloric Acid Salts of Compound I

### Example 1.1

### High Throughput Crystallization Screening

A stock solution was prepared by adding 0.50 g of Compound I (as crystals of the free base monohydrate) into a mixture of 20 mL BuOEtOH and 5 mL NMP. A first sample solution was prepared by adding 1 eq. of HCl to 120 mg of the stock solution (1 eq. HCl sample solution). A second sample solution was prepared by adding 2 eq. of HCl to 120 mg of the stock solution (2 eq. HCl sample solution). A sample solution of approximately ˜2 mg size were loaded into 48 wells of a 96 well plate (such that half the wells contained 1 eq. of HCl and half the plate contained 2 eq. of HCL). Solvent was removed using an evaporator for at least 12 hours. Next, a solvent was added to each of the wells and the wells were incubated at 40° C. for 1 hour. Generally, solvents included DMF, NMP, DMA, HOAc, MeOH, EtOH, THF, DCM, n-BuOH, BuOEtOH, acetone, and DME. For wells containing DMF, NMP, DMA, HOAc, MeOH, and EtOH, an equivolume of anti-solvent selected from water, iPrOH, and nBuOAc was added. For wells containing THF, DCM, n-BuOH, BuOEtOH, acetone, and DME, an equivolume of anti-solvent selected from heptane, iPrOH, and nBuOAc was added. For each of the solvents, one well did not receive any anti-solvent. Next, the samples were incubated for 72 hours at 40° C. The contents of the wells were analyzed by light microscopy and Raman microspectroscopy.

The procedure described above was repeated using a sample solution prepared by adding 2 eq. of HCl to 50 mg of the stock solution (2 eq. HCl sample solution).

The following crystalline forms of hydrochloric acid salts were prepared by high throughput crystallization:

| Form | HCl/Solvent | Counterion | Solvate |
|---|---|---|---|
| I. 1 | 1 eq. HCl in EtOH | 1 eq. HCl by EA | 0.4 eq. EtOH, |
| CA-2 | | | 0.8 eq. water |
| I. 2 | 1 eq. HCl in HOAc/ | 1 eq. HCl by EA | 1.6 eq. HOAc |
| HAC2-1 | nBuOAc | | 0.5 eq. water |
| I. 3 | 2 eq. HCl in nBuOAc | 2 eq. HCl in | Slurry sample |
| | | isolated solids | (Converts to I. 4) |
| I. 4 | 2 eq. HCl in HOAc/ | 2 eq. HCl by EA | 1 eq. water by KF |
| | nBuOAc | | |
| I. 5 | 2 eq. HCl in HOAc | 2 eq. HCl in | Slurry sample |
| | | isolated solids | (Converts to I. 4) |
| I. 6 | 2 eq. HCL in EtOH/ | 2 eq. HCl by EA | 0.1 eq. EtOH |
| H3-1 | water | | 0.8 eq. water |
| I. 7 | 1 eq. HCl in DMA/ | nm* | 1 eq. iPrOH |
| | iPrOH | | |

*nm—not measured

Larger quantities of certain hydrochloric acid salts were prepared according to the following procedures:

### Example 1.2

### Form I.1: Mono-hydrochloric Acid Salt

A slurry was prepared by adding 13.3 g of crystals of Compound I (butanolate) into 200 ml of 88.1% EtOH/4.7% MeOH/7.2% H₂O. To this slurry, 2.5 g HCl solution (37%) was added. The slurry became thin upon HCl addition, but thickened considerably within the next 2 minutes. The slurry was mixed at room temperature for approximately 72 hours. Next, the slurry was filtered in a Buchner funnel, and the wet cake was washed with 45 ml absolute EtOH. The wet cake was placed in a vacuum oven at 40° C. for approximately 20 hours, until a constant weight obtained. The dried material weight was 8.6 g. Analysis: 1 eq. HCl; 0.803 eq. water; and 0.36 eq. EtOH.

Elemental Analysis:

| | % C | % H | % N | % S | % Cl | % Water* |
|---|---|---|---|---|---|---|
| Observed | 48.71 | 5.34 | 17.58 | 5.77 | 12.88 | 2.60 |
| Calculated | 49.12 | 5.58 | 17.65 | 5.77 | 12.76 | 2.60 |

*% Water was determined using Karl Fischer analysis.

### Example 1.3

### Form I.2: Mono-hydrochloric Acid Salt

A mixture was prepared by dissolving 200 mg of Compound I into 1 mL of NMP. Next, 31.5 μL of concentrated HCl (1 eq.) was added. Solvent was removed by evaporation. Next, 1.5 mL of HOAc and 1.5 mL of butyl acetate were added. The mixture was stirred at ambient temperature for 5 days, heated to 50° C. for approximately 12 hours, then cooled to ambient temperature and isolated by filtration. Analysis: 1.0 eq. of HCl, 0.5 eq. of water, and 1.6 eq. of HOAc is present by EA.

| | % C | % H | % N | % S | % Cl | % Water* |
|---|---|---|---|---|---|---|
| Observed | 48.28 | 5.45 | 15.37 | 5.12 | 11.56 | 1.52 |
| Calculated | 48.07 | 5.51 | 15.58 | 5.09 | 11.26 | 1.43 |

*% Water was determined using Karl Fischer analysis.

### Example 1.4

### Form I.3, I.4: Di-hydrochloric Acid Salt

A mixture was prepared by dissolving 200 mg of Compound I into 1 mL of NMP, followed by the addition of 63 μL of concentrated HCl. Solvent was removed by evaporation, followed by the addition of 4 mL of HOAc and 4 mL of BuOAc. The mixture was stirred at ambient temperature for 5 days, stirred at 50° C. for approximately 12 hours, and then cooled to ambient temperature. A sample of the slurry was packed into a capillary tube and assayed by PXRD to afford form I.3. The remaining sample was isolated by filtration and dried to afford I.4 Analysis: 1.9 eq. HCl and 1 eq. water.

Elemental Analysis:

| | % C | % H | % N | % S | % Cl | % Water |
|---|---|---|---|---|---|---|
| Observed | 46.02 | 5.03 | 16.85 | 5.44 | 18.13 | 3.08 |
| Calculated | 45.93 | 5.24 | 17.05 | 5.57 | 17.87 | 3.13 |

### Example 1.5

### Form I.5, I.4: Di-hydrochloric Acid Salt

A mixture was prepared by adding 0.30 g of Compound I to 10 mL of HOAc, followed by the addition of 2 eq. of concentrated HCl. The mixture was seeded with Form I.4 seed crystals. The mixture was stirred for approximately 12 hours a small sample was packed into a capillary tube and identified as Form I.5. The remaining material was then filtered to afford 110 mg of crystalline material Form I.4. Analysis: 2 eq. HCl and 0.585 eq. H₂O.

Elemental Analysis:

| | % C | % H | % N | % S | % Cl | % Water |
|---|---|---|---|---|---|---|
| Observed | 46.00 | 5.50 | 16.98 | 5.59 | 18.79 | 1.84 |
| Calculated | 46.24 | 5.15 | 17.16 | 5.61 | 18.61 | 1.84 |

### Example 1.6

### Form I.6: Di-hydrochloric Acid Salt

A mixture was prepared by adding 13.5 g of crystals of Compound I (butanolate) to 176 mL EtOH and 23 mL water. To the mixture was added 4.04 g of concentrated HCl (2 eq). The mixture was stirred for 24 hours at ambient temperature, and then filtered. The resulting solid material was dried to afford 7.6 g crystalline material. Analysis: 2 eq. HCl and 0.83 eq. H₂O. GC: 0.48 w/w % EtOH.

Elemental Analysis:

| | % C | % H | % N | % S | % Cl | % Water |
|---|---|---|---|---|---|---|
| Observed | 45.97 | 5.17 | 17.17 | 5.52 | 18.58 | 2.59 |
| Calculated | 45.88 | 5.19 | 17.03 | 5.57 | 18.47 | 2.60 |

### Example 1.7

### Form I.7: Mono-hydrochloric Acid Salt

A mixture was prepared by adding 38 mg of Compound I into 5 mL of hot EtOH/water (5:1 v/v), followed by the addition of 1 eq. of HCl. Solvent was removed by evaporation over a period of 12 hours, followed by the addition of 400 μL of DMA and 400 μL of isopropanol. pNMR analysis detected 1 eq. of isopropanol relative to Compound I.

### Example 2

### Sulfuric Acid Salts of Compound I

### Example 2.1

### High Throughput Crystallization Screening

The following crystalline forms of sulfuric acid salts were prepared by high throughput crystallization, according to the general procedure described in Example 1.1.

| Form | Prep | Counterion | Solvate |
|---|---|---|---|
| II. 1 | 1 eq. H₂SO₄ in EtOH/ | 1 eq. H₂SO₄ by EA | 2 water by EA |
| | water | | |
| II. 2 | 1 eq. H₂SO₄ in | 0.67 eq. H₂SO₄ by | 3.2 eq. water by KF |
| | acetone/water | EA | |
| II. 3 | 1 eq. H₂SO₄ in | 1 eq. H₂SO₄ by EA | 1.5 water by EA |
| | acetone/water | | |
| II. 4 | 1 eq. H₂SO₄ in HOAc/ | 1 eq. H₂SO₄ by EA | 2 HOAc by EA 0.3 |
| | iPrOH | | water KF |
| II. 5 | 1 eq. H₂SO₄ in THF | 1 eq. H₂SO₄ by EA | 0.5 eq. water |
| SB-2 | | | THF |
| II. 6 | 0.5 eq. H₂SO₄ in | nm* | nm* |
| | DCM/iPrOH | | |
| II. 7 | 1 eq. H₂SO₄ in water | 0.7 eq. H₂SO₄ by | 1 eq. water by KF |
| | | EA | |
| II. 8 | 1 eq. H₂SO₄ in EtOH | 0.5 eq. H₂SO₄ | 0.5 eq. water |
| SA-1 | | | disordered EtOH |
| II. 9 | 1 eq. H₂SO₄ in DMF | 1 eq. H₂SO₄ | 0.5 eq. DMF |
| SC-1 | | | 0.5 eq. H₂O |
| II. 10 | 1 eq. H₂SO₄ in NMP | 0.5 eq. H₂SO₄ | 0.5 eq. water |
| SD-2 | | | 1 eq. NMP |

*not measured

Larger quantities of certain sulfuric acid salts were prepared according to the following procedures:

### Example 2.2

### Form II.1: Mono-sulfuric Acid Salt

A mixture was prepared by adding 0.5 g of Compound I to 10 mL EtOH. Next, a solution of 6.5 mL of 0.25 M sulfuric acid in water (1 eq.) at 50° C. was added. The mixture was seeded with Form II.1 (prepared by high throughput screening), stirred overnight at 50° C., and then allowed to cool to ambient temperature. The remaining solid material was isolated to afford 0.42 g of crystalline material. Analysis: Sulfuric acid salt comprising 1 eq. of sulfuric acid and 2 eq. of water per eq. of Compound I.

### Example 2.3

### Form II.2: Sulfuric Acid Salt

A mixture of 0.5 g of Compound I was added to 10 mL acetone and 2 mL water, followed by the addition of 56 μL (1 eq.) of concentrated sulfuric acid at 50° C. and 8 mL water. The mixture was stirred at 50° C. for 5 days and cooled to ambient temperature. The resulting solid material was filtered and dried to afford 0.47 g of crystalline material. Analysis: Sulfate salt comprising 0.67 eq. of sulfuric acid and 3.2 eq. of water per 1 eq. of Compound I.

Elemental Analysis:

| | % C | % H | % N | % S | % Cl | % Water |
|---|---|---|---|---|---|---|
| Observed | 43.01 | 5.38 | 15.68 | 8.65 | 5.89 | 9.46 |
| Calculated | 43.29 | 5.57 | 16.07 | 8.72 | 5.81 | 9.44 |

### Example 2.4

### Form II.3: Mono-sulfuric Acid Salt

A mixture was prepared by adding 0.5 g of Compound I to 10 mL acetone. Next, a solution of 6.5 mL of 0.25 M sulfuric acid in water (1 eq.) at 50° C. was added. The mixture was seeded with Form II.3 (prepared by high throughput screening), stirred overnight at 50° C., and then allowed to cool to ambient temperature. The resulting solid material was isolated and dried to afford 0.46 g of crystalline material. Analysis: Sulfate salt comprising 1 eq. of sulfuric acid and 1.5 eq. of water per eq. of Compound I.

Elemental Analysis:

| | % C | % H | % N | % S | % Cl | % Water |
|---|---|---|---|---|---|---|
| Observed | 42.82 | 4.86 | 15.66 | 10.49 | 5.77 | 4.05 |
| Calculated | 43.10 | 5.10 | 16.00 | 10.46 | 5.78 | 4.41 |

### Example 2.5

### Form II.4: Sulfuric Acid Salt

A mixture was prepared by adding 0.23 g of Compound I to 5 mL HOAc and 5 mL isopropanol, followed by the addition of 23 μL sulfuric acid. The mixture was heated to 50° C. for 15 minutes and then allowed to cool to ambient temperature. The mixture was seeded with crystalline material of Form II.1, which was prepared by high throughput screening. The mixture was stirred at ambient temperature for 5 days. The resulting crystalline material was isolated to afford 0.11 g of salt containing 1 eq. of sulfuric acid, 2 eq. of HOAc and 0.3 eq. of water per eq. of Compound I.

Elemental Analysis:

| | % C | % H | % N | % S | % Cl | % Water |
|---|---|---|---|---|---|---|
| Observed | 43.99 | 5.03 | 13.59 | 8.92 | 5.15 | 0.89 |
| Calculated | 43.86 | 5.19 | 13.77 | 9.01 | 4.98 | 0.81 |

### Example 2.6

### Form II.6: Sulfuric Acid Salt

A mixture was prepared by adding 42 mg of Compound I to 0.6 mL NMP, followed by the addition of 2.1 μL of concentrated sulfuric acid. Solvent was removed under vacuum at 40° C. Next, 0.5 mL DCM and 0.5 mL isopropanol were added. The mixture was stirred at 40° C. for 3 days. Crystalline material was isolated.

### Example 2.7

### Form II.7: Sulfuric Acid Salt

A mixture was prepared by adding 0.28 g of Compound I to 10 mL water. Next, 28 μL of concentrated sulfuric acid at 50° C. was added. The mixture was cooled to ambient temperature, and the resulting solid material was isolated by filtration and dried to afford 0.15 g of crystalline material. Analysis: Sulfate salt comprising 0.72 eq. of sulfuric acid and 1 eq. of water per eq. of Compound I.

Elemental Analysis:

| | % C | % H | % N | % S | % Cl | % Water |
|---|---|---|---|---|---|---|
| Observed | 46.14 | 5.17 | 16.63 | 9.30 | 6.22 | 3.18 |
| Calculated | 45.82 | 5.15 | 17.01 | 9.56 | 6.15 | 3.12 |

### Example 2.8

### Form II.8 (Form SA-1)

A mixture was prepared by adding 3 mg of Compound I (acetone/water solvate), 0.26 mL MeOH, and 0.04 mL of DMF. Next, 1 eq. of a 0.25 M sulfuric acid solution in EtOH was added. The solvent was removed under vacuum at 60° C. for 3 hours. Next, 100 μL EtOH was added and the mixture was allowed to stand at ambient temperature. Solvent was removed to afford crystalline material. A crystal was removed for single crystal analysis and found to be a mixed solvated/clathrate structure containing 0.5 H₂O and disordered EtOH.

### Example 2.9

### Form II.9 (Form SC-1)

A mixture was prepared by combining 1 g of Compound I and 8 mL of DMF and then heating to 100° C. Next, a total of 1 eq. of concentrated sulfuric acid and 2 mL water were added. The mixture was stirred at 95° C. and then allowed to cool to 70° C. Next, the mixture was allowed to cool to ambient temperature. The resulting solid material was isolated and dried to afford 0.32 g of crystalline material that was found to contain 0.5 eq. of sulfuric acid and 0.5 eq. of DMF.

### Example 2.10

### Form II.10 (Form SD-2)

A mixture was prepared by combining 1 g of compound I and 8 mL of NMP and then heating to 100° C. Next, 1 eq. of concentrated sulfuric acid and 2 mL water were added. The mixture was stirred at 95° C., and then allowed to ambient temperature. The resulting solid material was isolated and dried to afford 0.57 g of crystalline material that was found to contain 1 eq. of sulfuric acid, 1 eq. of NMP, and 0.5 eq. of water.

Elemental Analysis:

| | % C | % H | % N | % S | % Cl | % Water |
|---|---|---|---|---|---|---|
| Observed | 46.86 | 5.32 | 16.11 | 9.07 | 5.22 | 1.25 |
| Calculated | 46.71 | 5.52 | 16.14 | 9.24 | 5.11 | 1.30 |

### Example 3

### Methanesulfonic Acid Salts of Compound I

### Example 3.1

### High Throughput Crystallization Screening

The following crystalline forms of methanesulfonic acid salts were prepared by high throughput crystallization, according to the general procedure described in Example 1.1.

| Form | Prep | Counterion | Solvate |
|---|---|---|---|
| III. 1 | 1 eq. MSA in THF/ | 1 eq. MSA by pNMR | 0.3 eq. EtOAc |
| | EtOAc | | |
| III. 2 | 1 eq. MSA in 2- | 1 eq. MSA by pNMR | 0.2 eq. 2-BuOH, |
| | BuOH/MIBK | | 0.5 eq. MIBK |
| III. 3 | 1 eq. MSA in | 1 eq. MSA by pNMR | 0.75 eq. acetone |
| | EtOH/acetone | | |
| III. 4 | 1 eq. MSA in iPrOH | 1 eq. MSA by pNMR | 0.3 eq. iPrOH |
| III. 5 | 1 eq. MSA in DME | 1 eq. MSA by pNMR | 1.6 eq. DME |
| III. 6 | 1 eq. MSA in | 1 eq. MSA by pNMR | 0.2 eq. DME |
| | DME/nBuOAc | | |
| III. 7 | 1 eq. MSA in PG or | 1 eq. MSA | 1 eq. PG |
| PG-1 | PG/BuOAc | | |

Larger quantities of MSA salts were prepared according to the following general procedure:

### Example 3.2

### Form III.1

A mixture was prepared by adding 200 mg of Compound I and 1 eq. MSA to 4 mL of THF/EtOAc. The mixture was stirred at ambient temperature for 7 days, and then filtered and dried to afford crystalline material.

### Example 3.3

### Form III.2

A mixture was prepared by adding 200 mg of Compound I and 1 eq. MSA to 4 mL of 2-BuOH/MIBK. The mixture was stirred at ambient temperature for 7 days, and then filtered and dried to afford crystalline material.

### Example 3.4

### Form III.3

A mixture was prepared by adding 200 mg of Compound I and 1 eq. MSA to 4 mL EtOH/Acetone. The mixture was stirred at ambient temperature for 7 days, and then filtered and dried to afford crystalline material.

### Example 3.5

### Form III.4

A mixture was prepared by adding 200 mg of Compound I and 1 eq. MSA to 4 mL of iPrOH. The mixture was stirred at ambient temperature for 7 days, and then filtered and dried to afford crystalline material.

### Example 3.6

### Form III.5

A mixture was prepared by adding 200 mg of Compound I and 1 eq. MSA to 4 mL of DME. The mixture was stirred at ambient temperature for 7 days, and then filtered and dried to afford crystalline material.

### Example 3.7

### Form III.6

A mixture was prepared by adding 200 mg of Compound I and 1 eq. MSA to 4 mL of DME/BuOAc. The mixture was stirred at ambient temperature for 7 days, and then filtered and dried to afford crystalline material.

### Example 3.8

### Form III.7

A mixture was prepared by combining 4 g of Compound I, 10 mL of propylene glycol, and 1 eq. (513 μL) of MSA. The mixture was heated to 90° C. and cooled to ambient temperature. The resulting crystalline material was isolated and dried at 50° C. to afford 2.77 g (58 mol %)of a salt. Analysis: Methanesulfonate salt comprising 1 eq. methanesulfonic acid and 1 eq. of propylene glycol per eq. of Compound I.

Elemental Analysis:

| | % C | % H | % N | % S | % Cl | % Water |
|---|---|---|---|---|---|---|
| Observed | 47.50 | 5.73 | 14.79 | 9.60 | 5.49 | 0.49 |
| Calculated | 47.30 | 5.80 | 14.85 | 9.71 | 5.37 | — |

### Example 3.9

### Form III.7

A mixture was prepared by combining 10 g of Compound I and 1 eq. of MSA in 50 mL of propylene glycol. The mixture was heated to 65° C. and stirred until crystallization occurred. To the slurry, 50 mL of butyl acetate was added. The slurry was maintained at a temperature of 65° C. for 30 minutes and then cooled to ambient temperature. The resulting solid material was isolated and dried to afford 11.6 g (89 mol %) of crystalline material.

Elemental Analysis:

| | % C | % H | % N | % S | % Cl | % Water |
|---|---|---|---|---|---|---|
| Observed | 47.26 | 5.63 | 14.79 | 9.64 | 5.54 | 0.36 |
| Calculated | 47.30 | 5.80 | 14.85 | 9.71 | 5.37 | — |

### Example 4

### Phosphoric Acid Salts of Compound I

### Example 4.1

### High Throughput Crystallization Screening

The following crystalline forms of phosphoric acid salts were prepared by high throughput crystallization, according to the general procedure described in Example 1.1:

| Form | Prep | Counterion | Solvate |
|---|---|---|---|
| IV. 1 | 1.25 eq. H₃PO₄ in MeCN | 1.15 eq. H₃PO₄ by EA | 0.5 eq. water by KF |
| IV. 2 | 1.25 eq. H₃PO₄ in MeCN/ | 0.9 eq. H₃PO₄ by EA | 0.7 eq. MeCN by |
| | water | | NMR |
| | | | 1.5 eq. water by KF |
| IV. 3 | 1 eq. H₃PO₄ in | 0.67 eq. H₃PO₄ by EA | 0.67 eq. water by KF |
| | Acetone/water | | |
| IV. 4 | 1 eq. H₃PO₄ in MeCN | 0.9 eq. H₃PO₄ by EA | 2 eq. water by KF |
| IV. 5 | 1 eq. H₃PO₄ in MeCN | slurry, 0.9 eq. H₃PO₄ in | |
| | | isolated solids | |
| IV. 6 | 1 eq. H₃PO₄ in | 0.8 eq. H₃PO₄ by EA | 1.6 eq. water by KF |
| | EtOH/water | | |
| IV. 7 | 1 eq. H₃PO₄ in | slurry, 0.8 eq. H₃PO₄ in | |
| | EtOH/water | isolated solids | |
| IV. 8 | 0.5. eq. H₃PO₄ in | nm* | nm* |
| | DME/iPrOH | | |
| IV. 9 | 1 eq. H₃PO₄ in MeOH | nm* | nm* |
| IV. 10 | excess H₃PO₄ in DMA | 3 eq. H₃PO₄ | 1 eq. water |
| SA-1 | | | 0.5 eq. DMA |

*not measured

Larger quantities of certain phosphoric acid salts were prepared according to the following procedures:

### Example 4.2

### Form IV.1

A mixture was prepared by combing 0.5 g Compound I, 10 mL MeCN, and 1.25 eq. phosphoric acid. The mixture was stirred overnight at 50° C. and allowed to cool to ambient temperature. Crystalline material was isolated from the mixture, which comprised 1.15 eq. phosphoric acid and 0.5 eq. of water per eq. of Compound I.

### Example 4.3

### Form IV.2

A mixture was prepared by combing 0.5 g Compound I, 5 mL MeCN, 5 mL water, and 1.25 eq. phosphoric acid. The mixture was stirred overnight at 50° C. and allowed to cool to ambient temperature. Crystalline material was isolated from the mixture, which comprised 1 eq. phosphoric acid and 1.5 eq. water per eq. of Compound I.

### Example 4.4

### Form IV.3

A mixture was prepared by combing 0.51 g Compound I, 5 mL acetone, 5 mL water, and 1 eq. phosphoric acid. The mixture was stirred overnight at 45° C. and allowed to cool to ambient temperature. Crystalline material was isolated from the mixture.

### Example 4.5

### Form IV.4 and Form IV.5

A mixture was prepared by combing 0.53 g Compound I, 10 mL MeCN, and 1 eq. phosphoric acid. The mixture was stirred overnight at 50° C. and allowed to cool to ambient temperature. Material from the slurry was packed into a capillary tube and analyzed by PXRD to afford Form IV.5 (slurry). Crystalline material, 0.49 g, Form IV.4 was isolated from the mixture, and comprised 0.9 eq. phosphoric acid and 2 eq. water per eq. of Compound I.

### Example 4.6

### Form IV.6 and Form IV.7

A mixture was prepared by combing 0.37 g Compound I, 5 mL EtOH, 5 mL water, and 1 eq. phosphoric acid. The mixture was stirred overnight at 45° C. and allowed to cool to ambient temperature. Material from the slurry was packed into a capillary tube and to afford Form IV.7 (slurry). Crystalline material Form IV.6 (0.48 g) was isolated from the mixture, and comprised 0.8 eq. phosphoric acid and 1.6 eq. water per eq. of Compound I.

### Example 4.7

### Form IV.8

A mixture was prepared by combining 40 mg of Compound I, 1 mL 7/3 EtOH/water (v/v) and 0.5 eq. of concentrated phosphoric acid. The mixture was heated to 60° C. and then the solvent was removed. To the residue, 0.5 mL of iPrOH and 0.5 mL of DME were added. The mixture was stirred overnight and isolated to afford crystalline material.

### Example 4.8

### Form IV.9

A mixture was prepared by combining 40 mg of Compound I, 1 mL 7/3 EtOH/water and 1 eq. of concentrated phosphoric acid. The solvent was removed and 1 mL MeOH was added to the residue. The mixture was stirred overnight and isolated to afford crystalline material.

### Example 4.9

### Form IV-10

A solution was prepared by combining Compound I, DMA, and aqueous phosphoric acid at 80° C. Upon cooling, a sample was taken for single crystal analysis to afford Form IV.10. Analysis of the crystal structure found an average structure comprising 2 eq. of Compound I as the +2 ion, one eq. of H₃O⁺, 5 eq. of H₂PO₄⁻, one eq. of H₃PO₄, one eq. of H₂O, and DMA.

### Example 5

### Tartaric Acid Salts of Compound I

### Example 5.1

### High Throughput Crystallization Screening

The following crystalline forms of tartaric acid salts were prepared by high throughput crystallization, according to the general procedure described in Example 1.1:

| Form | Prep | Counterion | Solvate |
|---|---|---|---|
| V. 1 | 1 eq. D tartaric | 1 eq. D tartaric acid by | 0.16 water KF |
| | acid in EtOH | EA | |
| V. 2 | 1 eq. L tartaric | 1 eq. L tartaric acid by | 0.3 EtOH NMR |
| | acid in EtOH/water. | EA | 1.3 water KF |
| V. 3 | 1 eq. racemic tartaric | 1 eq. racemic tartaric | 0.5 eq. EtOH |
| | acid in EtOH | acid | |

Larger quantities of the tartaric acid salts were prepared according to the following procedures:

### Example 5.2

### Form V.1

A mixture was prepared by combining 0.82 g Compound I, 15 mL EtOH, and 250 mg D-tartaric acid. The mixture was heated to 70° C. to afford a thin slurry, cooled to 40° C., and maintained at 40° C. overnight. The mixture was cooled, and filtered. The solid material was dried to afford 0.90 g of crystalline material.

Elemental Analysis:

| | % C | % H | % N | % S | % Cl | % Water |
|---|---|---|---|---|---|---|
| Observed | 48.63 | 4.76 | 15.08 | 4.96 | 5.57 | 0.46 |
| Calculated | 48.71 | 5.08 | 15.30 | 5.00 | 5.53 | |

### Example 5.3

### Form V.2

A mixture was prepared adding 13.33 g of crystalline Compound I (butanolate, eq. to 10 g of Compound I) in 175 mL EtOH at 60° C. A solution of 0.37 g L-tartaric acid in 16 mL EtOH and 8 mL of water were added. The mixture was stirred at 60° C. for 14 hours, cooled to ambient temperature, and filtered. The solid material was dried to afford 13.7 g of crystalline material. Analysis: mono-L-tartrate salt comprising 1.33 eq. of water and 0.3 eq. EtOH.

Elemental Analysis:

| | % C | % H | % N | % S | % Cl | % Water |
|---|---|---|---|---|---|---|
| Observed | 47.51 | 5.49 | 14.70 | 4.86 | 5.39 | 3.55 |
| Calculated | 47.27 | 5.46 | 14.48 | 4.73 | 5.23 | 3.55 |

### Example 5.4

### Form V.3

A mixture was prepared by combining 0.225 g of racemic tartaric acid in 15 mL of ethanol followed by the addition of 1.0 g of Compound I. The mixture was heated to 50° C. and stirred overnight. The slurry was then cooled to ambient temperature and the resulting solid material was isolated and dried to afford 0.86 g of crystalline material that was found to contain 1 eq. of racemic tartaric acid and 0.5 eq. of ethanol.

### Example 6

### Benzoic Acid Salts of Compound I

### Example 6.1

### High Throughput Crystallization Screening

The following crystalline form of a benzoic acid salt was prepared by high throughput crystallization, according to the general procedure described in Example 1.1:

| Form | Prep | Counterion | Solvate |
|---|---|---|---|
| VI. 1 | 1 eq. benzoic acid in toluene | 1 eq. benzoic acid | none |
| VI. 2 | 1 eq. benzoic acid in toluene | slurry | — |

Larger quantities of the above benzoic acid salts were prepared according to the following procedure:

### Example 6.2

### Form VI.1 and VI.2

A mixture was prepared by combining 0.33 g Compound I, 5 mL EtOH, 0.6 mL of water, and a solution of 1 eq. of benzoic acid in 1 mL EtOH at 80° C. Solvent was removed by evaporation under vacuum. Next, 10 ml toluene was added and a seed crystal of Form VI.1, which was prepared in the high throughput crystallization study, was added. The mixture was stirred for 5 days at ambient temperature. A slurry sample was packed into a capillary and analyzed by PXRD to afford Form VI.2. Crystalline material was isolated from the mixture and dried to afford Form VI.1.

### Example 7

### Fumaric Acid Salts of Compound I

### Example 7.1

### High Throughput Crystallization Screening

The following crystalline forms of fumaric acid salts were prepared by high throughput crystallization, according to the general procedure described in Example 1.1:

| Form | Prep | Counterion | Solvate |
|---|---|---|---|
| VII. 1 | 1 eq. fumaric acid in iPrOH | 1 eq. fumaric acid by | 0.16 eq. iPrOH by NMR |
| | | NMR | |
| VII. 2 | 1 eq. fumaric acid in acetone | 1 eq. fumaric acid by | 1 eq. acetone by NMR |
| | | NMR | |
| VII. 3 | 1 eq. fumaric acid in | 1 eq. fumaric acid by | 0.3 eq. BuOAc by NMR |
| | BuOAc | NMR | |
| VII. 4 | 1 eq. fumaric acid in | 1 eq. fumaric acid by | 0.3 eq. MIBK by NMR |
| | MIBK/heptane | NMR | |
| VII. 5 | 1 eq. fumaric acid in | 1 eq. fumaric acid by | |
| | MeOH | NMR | |
| VII. 6 | 1 eq. fumaric acid in toluene | 0.5 eq. fumaric acid | 1 eq. toluene |
| TO-1 | | | |
| VII. 7 | 0.5 fumaric acid in | 0.5 eq. fumaric acid by | |
| | DCM/heptane | NMR | |
| VII. 8 | 0.5 eq. fumaric acid | 0.5 eq. fumaric acid by | 0.8 eq. MIBK |
| | in MIBK/heptane | NMR | |
| VII. 9 | 0.5 eq. fumaric acid | 0.5 eq. fumaric acid by | |
| | in DCM/heptane | NMR | |
| VII. 10 | 0.5 eq. fumaric acid | slurry sample | |
| | in DCM/heptane | | |
| VII. 11 | 0.5 eq. fumaric acid | 0.5 eq. fumaric acid by | 0.3 eq. heptane |
| | in heptane | NMR | |
| VII. 12 | 0.5 eq. fumaric acid | 0.5 eq. fumaric acid by | |
| | in MeOH/water | NMR | |
| VII. 13 | 0.5 eq. fumaric acid | slurry sample | |
| | in MeOH/water | | |

Larger quantities of certain fumaric acid salts were prepared according to the following procedures:

### Example 7.2

### Form VII.1

A mixture was prepared by combining 40 mg of Compound I, 1 mL of a solution of 90% EtOH/10% water, and 1 eq. of fumaric acid. Solvent was removed.

Next, 1 mL of isopropanol was added. The resulting mixture was heated and maintained at 40° C. for 4 days. The resulting solid material was isolated and dried to afford crystalline material.

### Example 7.3

### Form VII.2

A mixture was prepared by combining 0.6 g of Compound I, 20 mL of 7/3 EtOH/water solution (v/v), and 1 eq. of fumaric acid. Solvent was removed. Next, 10 mL of acetone was added. The resulting mixture was heated and maintained at a temperature of 40° C. overnight. The resulting solid material was isolated and dried to afford 0.60 g of crystalline material.

### Example 7.4

### Form VII.3

A mixture was prepared by combining 0.6 g Compound I, 20 mL of 7/3 EtOH/water solution (v/v), and 1.0 eq. fumaric acid. Solvent was removed under vacuum. Next, 10 mL of BuOAc was added. The resulting mixture was heated and maintained at a temperature of 40 ° C. overnight. The resulting solid material was isolated and dried to afford 0.64 g of crystalline material.

### Example 7.5

### Form VII.4

A mixture was prepared by combining 0.6 g of Compound I, 20 mL of 7/3 EtOH/water solution (v/v), and 1 eq. of fumaric acid. Solvent was removed. Next, 5 mL of MIBK and 5 mL of heptane was added. The resulting mixture was heated and maintained at a temperature of 40° C. overnight. The resulting solid material was isolated and dried to afford 0.69 g of crystalline material.

### Example 7.6

### Form VII.5

A mixture was prepared by combining 0.6 g Compound I, 20 mL of 7/3 EtOH/water solution (v/v), and 1.0 eq. fumaric acid. Solvent was removed under vacuum. Next, 10 mL MeOH was added and the resulting mixture was heated and maintained at a temperature of 40° C. overnight. The resulting solid material was isolated and dried to afford 0.52 g of crystalline material.

### Example 7.7

### Mixture of Form VII.5 and Form VII.6

A mixture was prepared by combining 0.6 g Compound I, 20 mL of 7/3 EtOH/water solution (v/v), and 1.0 eq. fumaric acid. Solvent was removed under vacuum. Next, 10 mL toluene was added and the resulting mixture was heated and maintained at a temperature of 40° C. overnight. The resulting solid material was isolated and dried to afford crystalline material that is a mixture of Form VII.5 and VII.6.

### Example 7.8

### Form VII.6

A mixture was prepared by combining the Form VII.13 slurry and several drops of toluene. Slow evaporation under ambient conditions afforded samples for single crystal analysis that identified From VII.6, which comprised 1 eq. of toluene and 0.5 eq. of fumaric acid per eq. of compound I.

### Example 7.9

### Form VII.7

A mixture was prepared by combining 40 mg of Compound I, 1 mL of 9/1 EtOH/water solution (v/v), and 0.5 eq. of fumaric acid. Solvent was removed. Next, 1 mL of 1/1 DCM/heptane solution (v/v) was added. The resulting mixture was heated and maintained at a temperature of 40° C. for a period of 4 days. The resulting solid material was isolated and dried to afford crystalline material.

### Example 7.10

### Form VII.8

A mixture was prepared by combining 0.6 g Compound I, 20 mL of 7/3 EtOH/water solution (v/v), and 0.5 eq. fumaric acid. Solvent was removed under vacuum. Next, 10 mL of 1/1 MIBK/heptane solution (v/v) was added. The resulting mixture was heated and maintained at a temperature of 40° C. overnight. The resulting solid material was isolated and dried to afford 0.68 g of crystalline material.

### Example 7.11

### Form VII.9 and Form VII.10

A mixture was prepared by combining 0.6 g of Compound I, 20 mL of 7/3 EtOH/water solution (v/v), and 0.5 eq. fumaric acid. Solvent was removed under vacuum. Next, 10 mL of 1/1 DCM/heptane solution (v/v) was added. The resulting mixture was heated and maintained at a temperature of 40° C. overnight. The resulting solid material was isolated and dried to afford 0.56 g of crystalline material VII.9. A sample of the slurry was packed into a capillary tube and identified by PXRD as crystalline material VII.10.

### Example 7.12

### Form VII.11

A mixture was prepared by combining 0.6 g Compound I, 20 mL of 7/3 EtOH/water solution (v/v), and 0.5 eq. fumaric acid. Solvent was removed under vacuum. Next, 10 mL of heptane was added and the resulting mixture was heated and maintained at a temperature of 40° C. overnight. The resulting solid material was isolated and dried to afford 0.36 g of crystalline material.

### Example 7.13

### Form VII.12 and Form VII.13

A mixture was prepared by combining 0.6 g Compound I, 20 mL of 7/3 EtOH/water solution (v/v), and 0.5 eq. fumaric acid. Solvent was removed under vacuum. Next, 10 mL of 1/1 MeOH/water solution (v/v) was added. The resulting mixture was heated and maintained at a temperature of 40° C. overnight. The resulting solid material was isolated and dried to afford 0.44 g of crystalline material Form VII.12. A sample of the slurry was packed into a capillary tube and analyzed by PXRD as crystalline material Form VII.13.

### Example 8

### Maleic Acid Salts of Compound I

### Example 8.1

### High Throughput Crystallization Screening

The following crystalline forms of maleic acid salts were prepared by high throughput crystallization, according to the general procedure described in Example 1.1:

| Form | Prep | Counterion | Solvate |
|---|---|---|---|
| VIII. 1 | 1 eq. maleic acid in | 1 eq. maleic acid by NMR | |
| | MeCN | | |
| VIII. 2 | 1 eq. maleic acid in | 1 eq. maleic acid slurry | |
| | MeCN | sample | |
| VIII. 3 | 1 eq. maleic acid in | 1 eq. maleic acid by SC | 0.9 eq. water by KF |
| H3-2 | water | | |
| VIII. 4 | 1 eq. maleic acid in | 1 eq. maleic acid by | 0.5 eq. acetone by NMR |
| | acetone | NMR | |
| VIII. 5 | 1 eq. maleic acid in | 1 eq. maleic acid by | |
| | heptane | NMR | |
| VIII. 6 | 1 eq. maleic acid in | 1 eq. maleic acid | 1 eq. EtOH |
| E-1 | EtOH/water | | |
| VIII. 7 | 0.5 eq. maleic acid | 0.5 eq. maleic acid by | 0.5 eq. EtOH |
| | in EtOH | NMR | |
| VIII. 8 | 0.5 eq. maleic acid in | 0.5 eq. maleic acid by | 0.5 eq. toluene |
| | toluene | NMR | |

Larger quantities of certain maleic acid salts were prepared according to the following procedures:

### Example 8.2

### Form VIII.1 and Form VIII.2

A mixture was prepare by combining 4 g of Compound I, 100 mL of MeCN, and 0.91 g of maleic acid. Next, the mixture was seeded with seed crystals of Form VIII.1 from the high throughput crystallization and stirred at 80° C. for 2 hours.

A sample of the slurry was packed into a capillary tube and Form VIII.2 was observed by PXRD. The mixture was cooled to 5° C. and the resulting solid material was isolated by filtration to afford 4.40 g of crystalline material. Analysis: mono-maleate salt of Compound I comprising 2.86% water (0.87 eq.) Form VIII.1

Elemental Analysis:

| | % C | % H | % N | % S | % Cl | % Water |
|---|---|---|---|---|---|---|
| Observed | 50.38 | 5.10 | 15.71 | 5.14 | 5.76 | 2.54 |
| Calculated | 50.38 | 5.16 | 15.82 | 5.17 | 5.72 | 2.53 |

### Example 8.3

### Form VIII.3

A mixture was prepared by combining 750 mg of Compound I and 1 eq. of acetic acid in 15 mL of water and heating to reflux. One molar equivalent of maleic acid was charged and the mixture was cooled to ambient temperature. The resulting solid material was isolated and dried to afford 742 mg of crystalline material. Analysis: 1 eq. maleic acid and 0.88 eq. of water per eq. of Compound I.

Elemental Analysis:

| | % C | % H | % N | % S | % Cl | % Water |
|---|---|---|---|---|---|---|
| Observed | 50.26 | 5.02 | 15.83 | 5.17 | 5.79 | 2.58 |
| Calculated | 50.37 | 5.16 | 15.82 | 5.17 | 5.72 | 2.56 |

### Example 8.4

### Form VIII.4

A mixture was prepared by combining 40 mg Compound I, 1.25 mL of 5/1 EtOH/water solution (v/v), and 1 eq. of maleic acid. Solvent was removed under vacuum. Next, 1 mL acetone was added. The resulting mixture was maintained at ambient temperature for a period of one week. The resulting solid material was isolated and dried to afford crystalline material.

### Example 8.5

### Form VIII.5

A mixture was prepared by combining 40 mg Compound I, 1.25 mL of 5/1 EtOH/water solution (v/v), and 1 eq. of maleic acid. Solvent was removed under vacuum. Next, 1 mL of heptane was added and the resulting mixture was maintained at ambient temperature for 1 week. The resulting solid material was isolated and dried to afford crystalline material.

### Example 8.6

### Form VIII.6

A mixture was prepared by combining 5 mg Compound I, Form VIII.3, and 0.1 mL of EtOH/water (1/1 v/v) and allowing the solution to evaporate overnight. A single crystal was collected for analysis.

### Example 8.7

### Form VIII.7

A mixture was prepared by combining 40 mg Compound I, 1.25 mL of 5/1 EtOH/water solution (v/v), and 0.5 eq. of maleic acid. Solvent was removed under vacuum. Next, 1 mL EtOH was added and the resulting mixture was maintained at ambient temperature for a period of one week. The resulting solid material was isolated and dried to afford crystalline material.

### Example 8.8

### Form VIII.8

A mixture was prepared by combining 40 mg Compound I, 1.25 mL of 5/1 EtOH/water solution (v/v), and 0.5 eq. of maleic acid. Solvent was removed under vacuum. Next, 1 mL of toluene was added and the resulting mixture was maintained at ambient temperature for a period of 1 week. The resulting solid material was isolated and dried to afford crystalline material.

### Example 9

### Malic Acid Salt of Compound I

### Example 9.1

### High Throughput Crystallization Screening

The following crystalline form of a malic acid salt was prepared by high throughput crystallization, according to the general procedure described in Example 1.1:

| Form | Prep | Counterion | Solvate |
|---|---|---|---|
| IX. 1 | 1 eq. malic acid in | 1 eq. malic acid by | 0.4 eq. EtOH by NMR |
| | EtOH/water | NMR | |

A larger quantity of the above malic acid salt was prepared according to the following procedure:

### Example 9.2

### Form IX.1

A mixture was prepared by combining 1 g Compound I, 20 mL EtOH, and 5 mL water. The mixture was heated to 80° C. Next, 1 eq. of L-malic acid was added. The mixture was stirred at 50° C. for one day. The resulting solid material was isolated and dried at 50° C. for 4 days to afford 0.58 g of crystalline material. The L-malic acid salt comprised 0.4 eq. EtOH.

### Example 10

### Hydrobromic Acid Salt of Compound I

### Example 10.1

### High Throughput Crystallization Screening

The following crystalline form of a hydrobromic acid salt was prepared by high throughput crystallization, according to the general procedure described in

### Example 1.1:

| Form | Prep | Counterion | Solvate |
|---|---|---|---|
| X. 1 | 1 eq. HBr in EtOH | 1 eq. HBr by EA | 1 eq. water by KF |
| H1.5-1 | | | |

A larger quantity of the above hydrobromic acid salt was prepared according to the following procedure:

### Example 10.2

### Form X.1 (Form H1.5-1)

A mixture was prepared by combining 4 g of Compound I, 100 mL EtOH, and 0.89 mL of concentrated hydrobromic acid. The mixture was seeded with crystals of Form X.1 (grown from iPrOH) and stirred at 80° C. for 2 hours. The mixture was cooled to 5° C. and the resulting solid material was isolated by filtration to afford 3.48 g of crystalline material. Analysis: mono-HBr salt of Compound I comprising 2.94% water (1 eq.).

Elemental Analysis:

| | % C | % H | % N | % S | % Cl | % Br | % Water |
|---|---|---|---|---|---|---|---|
| Observed | 45.25 | 4.97 | 16.93 | 5.68 | 6.40 | 13.36 | 2.94 |
| Calculated | 45.02 | 4.98 | 16.70 | 5.46 | 6.04 | 13.61 | 3.07 |

### Example 11

### Benzenesulfonic Acid Salts of Compound I

### Example 11.1

### High Throughput Crystallization Screening

The following crystalline forms of benzenesulfonic acid salts were prepared by high throughput crystallization, according to the general procedure described in Example 1.1:

| Form | Prep | Counterion | Solvate |
|---|---|---|---|
| XI. 1 | 1 eq. BSA in THF | 1 eq. BSA | 1 eq. THF |
| XI. 2 | 1 eq. BSA in MIBK | 1 eq. BSA | 1 eq. MIBK |
| XI. 3 | 1 eq. BSA in MIBK | 1 eq. BSA | |

Larger quantities of certain benzenesulfonic acid salts were prepared according to the following procedures:

### Example 11.2

### Form XI.1

A mixture was prepared by combining 40 mg Compound I, 1.5 mL of 7/3 EtOH/water solution (v/v), and 1 eq. of BSA. Solvent was removed under vacuum. Next, 1 mL of THF was added. The resulting mixture was maintained at ambient temperature for at least 4 days. The resulting solid material was isolated and dried to afford crystalline material.

Elemental Analysis:

| | % C | % H | % N | % S | % Cl | % Water |
|---|---|---|---|---|---|---|
| Observed | 52.13 | 4.85 | 15.09 | 9.53 | 5.45 | <0.1 |
| Calculated | 52.04 | 4.99 | 15.17 | 9.92 | 5.48 | |

### Example 11.3

### Form XI.2

A mixture was prepared by combining 0.5 g of Compound I, 5 mL EtOH, 2.5 mL of water, and a solution of 150 mg of BSA in 1.5 mL of water. The mixture was heated to 75° C. to dissolve Compound I. Solvent was removed on a roto-evaporator. The solid material was resuspended in MIBK. Solvent was removed. Next, the solid material was resuspended in 1:1 iPrOH/MIBK (v/v). The solvent was removed. Then, 10 mL of MIBK and crystal seed (seed was grown in BuOAc/heptane) were added. The resulting mixture was stirred at ambient temperature overnight. The resulting solid material was isolated and dried to afford 0.47 g of crystalline material.

### Example 11.4

### Form XI.3

A mixture was prepared by combining 40 mg of Compound I, 1.5 mL of 7/3 EtOH/water solution (v/v), and 1 eq. of BSA. Solvent was removed under vacuum. Next, 1 mL of MIBK was added. The resulting mixture was maintained at ambient temperature for at least 4 days. The resulting solid material was isolated and dried to afford crystalline material.

### Example 12

### Citric Acid Salt of Compound I

### Example 12.1

### High Throughput Crystallization Screening

The following crystalline form of a citric acid salt was prepared by high throughput crystallization, according to the general procedure described in Example 1.1:

| Form | Prep | Counterion | Solvate |
|---|---|---|---|
| XII. 1 | 0.5 eq. citric acid | 0.33 eq. citric acid by EA | 1 eq. EtOH |
| | in EtOH | | 1.25 eq. water |

Larger quantities of the citric acid salt was prepared according to the following procedure:

### Example 12.2

### Form XII.1

A mixture was prepared by combining 0.5 g of Compound I and 0.5 eq. of citric acid in 10 mL of 7/3 EtOH/water solution (v/v). The mixture was heated to 70° C., and then cooled to ambient temperature. Solvent was removed under vacuum. Next, 10 mL EtOH was added and the resulting mixture was stirred at 50° C. for 16 hours. The resulting solid material was isolated and dried to afford 0.55 g of crystalline material.

### Example 13

### Acetic Acid Salts of Compound I

### Example 13.1

### High Throughput Crystallization Screening

The following crystalline forms of acetic acid salts were prepared by high throughput crystallization, according to the general procedure described in Example 1.1:

| Form | Prep | Counterion | Solvate |
|---|---|---|---|
| XIII. 1 | 1 eq. acetic acid in MIBK | 1 eq. acetic acid | 1 eq. MIBK |
| XIII. 2 | acetic acid/NMP | 1 eq. acetic acid by | 1 eq. NMP |
| NMP-1 | | SC | |

A larger quantities of the above acetic acid salts were prepared according to the following procedures:

### Example 13.2

### Form XIII.1

A mixture was prepared by combining 40 mg of Compound I, 1.1 mL of 7/3 EtOH/water solution (v/v), and 1 eq. of HOAc. Solvent was removed under vacuum. Next, 1 mL of MIBK was added, and the resulting mixture was stirred at ambient temperature for at least 4 days. The resulting solid material was isolated and dried to afford crystalline material.

### Example 13.3

### Form XIII.2 (Form NMP-1)

A solution was prepared by taking a slurry of Form I.2 (mono-HCl, di-acetate solvate in butyl acetate/acetic acid containing residual NMP) and adding a small amount of water. The mixture was dried under ambient conditions afforded crystalline material.

### Example 14

### P-Toluenesulfonic Acid Salts of Compound I

The following crystalline forms of p-toluenesulfonic acid (pTSA) salts were prepared by crystallization studies described below:

| Form | Prep | Counterion |
|---|---|---|
| XIV. 1 | 1 eq. pTSA in EtOH | 1 eq. pTSA |
| XIV. 2 | 1 eq. pTSA in EtOH | 1 eq. pTSA |
| N-1 | | |

Larger quantities of pTSA salts were prepared according to the following procedures:

### Example 14.1

### Form XIV.1

A mixture was prepared by adding 0.75 g of Compound I (contained 10% NMP) and 0.292 g of pTSA to 5 mL EtOH and heating to reflux. After crystallization occurred, 1 7.5 mL EtOH was added. The mixture was maintained at a temperature in the range of from 65-70° C. for 4 hours, then cooled to ambient temperature, and maintained at ambient temperature for 16 hours. The resulting solid material was isolated and dried to afford 0.85 g of crystalline material.

### Example 14.2

### Form XIV.2

A mixture was prepared by adding 0.75 g of Compound I and 281 mg of pTSA to 15 mL EtOH, and then heating to reflux. The mixture was cooled to ambient temperature and 0.89 g of crystalline material was afforded.

Elemental Analysis:

| | % C | % H | % N | % S | % Cl | % Water |
|---|---|---|---|---|---|---|
| Observed | 52.71 | 4.74 | 14.68 | 9.45 | 5.34 | <0.1% |
| Calculated | 52.92 | 4.90 | 14.89 | 9.74 | 5.38 | |

### Example 15

### Gentisic Acid Salts of Compound I

The following crystalline forms of gentisic acid salts (2,5-dihydroxybenzoic acid) were prepared:

| Form | Prep | Counterion | Solvate |
|---|---|---|---|
| XV. 1 | 1 eq. gentisic acid in EtOH/water | 1 eq. gentisic | 0.5 eq. water |
| XV. 2 | 1 eq. gentisic acid in water | acid | |

Larger quantities of the gentisic acid salts were prepared according to the following procedures:

### Example 15.1

### Form XV.1

A mixture was prepared by adding 0.75 g of Compound I and 228 mg of gentisic acid to 15 mL of a 4/1 water-EtOH solution (v/v). The mixture was heated to reflux and then cooled to ambient temperature for 16 hours. The mixture was filtered and 0.81 g of crystalline material was afforded. Analysis: 1 eq. gentisic acid and 0.5 eq. water per eq. of Compound I.

Elemental Analysis:

| | % C | % H | % N | % S | % Cl | % Water |
|---|---|---|---|---|---|---|
| Observed | 53.53 | 4.91 | 15.13 | 4.88 | 5.56 | 1.22 |
| Calculated | 53.49 | 5.11 | 15.06 | 4.92 | 5.44 | 1.38 |

### Example 15.2

### Form XV.2

An acetic acid salt solution was prepared containing 50 mg/mL Compound I solution in 2.85 equivalents of acetic acid. Next, 0.9 mL of the acetic acid salt solution was combined with 1 mL of a solution of 0.1 M gentisic acid in water. The resulting mixture was heated to 100° C. and then cooled to ambient temperature. The mixture was maintained at ambient temperature for 15 days. The resulting solid material was isolated by centrifugation filter to afford 241 mg wet crystalline material.

### Example 16

### Salicylic Acid Salt of Compound I

The following crystalline form of a salicylic acid salt was prepared by crystallization studies described below.

| Form | Prep | Counterion | Solvate |
|---|---|---|---|
| XVI. 1 | 1 eq. salicylic | 1 eq. salicylic acid | neat |
| SS-2 | acid in EtOH | | |

A mixture was prepared by adding 0.75 g of Compound I and 204 mg of salicylic acid to 15 mL EtOH and heating to reflux. The mixture was cooled to ambient temperature and 0.53 g of crystalline material was isolated.

### Example 17

### p-Acetamidobenzoic Acid Salt of Compound I

The following crystalline form of a p-acetamidobenzoic acid salt was prepared by crystallization studies described below.

| Form | Prep | Counterion | Solvate |
|---|---|---|---|
| XVII. 1 | 1 eq. p-acetamidobenzoic | 1 eq. p-acetamidobenzoic | 0.5 eq. |
| | acid in EtOH | acid | EtOH |

A mixture was prepared by dissolving 0.81 g of Compound I into 1.5 mL of an aqueous solution comprising 2.8 eq. of acetic acid and 1.33 mL of 0.125 M acetamidobenzoic acid. The mixture was stirred for 30 minutes, filtered and the resulting solid material was washed with water to afford 0.89 g of wet solid material.

### Examples 18-20

### Preparation of Uncoated Tablets Comprising Compound I

Granulation for uncoated tablets was prepared by combining with mixing the intra-granular materials listed in Table 1 in a high shear mixer/granulator. The premixed powder was granulated with water added at a controlled rate (4 g/min) in a high shear granulator until complete addition of water (Table 1). The wet-granulated mass was dried at 50° C. to a moisture content of about 3 weight % (Loss on Drying, LOD) in an oven. The dried granules were then screened thorough a #18 mesh. The remaining portion of croscarmellose sodium (extra-granular) was added with (Example 18) or without the organic acid (Examples 19 and 20) to the screened granules in a diffusion mixer and mixed. Magnesium stearate was added to the blend in the diffusion mixer and mixed to give the final blend. The final blend was compressed on a tablet press into 20-mg strength tablets (80-mg tablet weight) to a target hardness of 7 SCU (Strong Cobb Units).

TABLE 1

| | | | Example |
|---|---|---|---|
| Material | Example 18 | Example 19 | 20 |
| Intra-granular: | | | |
| Lactose, monohydrate powder | 10.39 g | 10.39 g | 10.39 g |
| Compound I | 10.41 | 10.41 | 10.41 |
| Tartaric acid | — | 5.20 | — |
| Citric acid | — | — | 5.2 |
| Microcrystalline cellulose | 10.80 | 10.80 | 10.80 |
| Croscarmellose sodium | 0.80 | 0.80 | 0.80 |
| Hydroxypropyl cellulose, EXF | 1.20 | 1.20 | 1.20 |
| Water | 17 | 8 | 9.6 |
| Extra-granular: | | | |
| Croscarmellose sodium | 0.693 | 0.736 | 0.711 |
| Tartaric acid | 4.503 | — | — |
| Magnesium stearate | 0.34 | 0.368 | 0.355 |

Twenty tablets each of Examples 18 to 20 were placed in 100 ml high density polyethylene (HDPE) bottles for stability studies. The bottles were stored at various temperature and humidity conditions to evaluate the stability of the tablets.

What is claimed is:

1. A pharmaceutical composition comprising:
a) Compound I of formula: 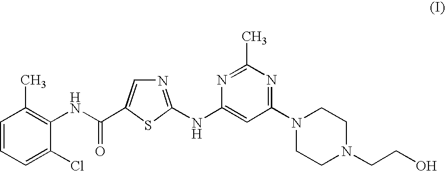
and at least one acid pH modifier;
and/or
b) a pharmaceutically-acceptable acid salt of Compound I and one or more pharmaceutically-acceptable excipients.

2. The pharmaceutical composition of claim 1 comprising from about 1 to about 50 weight % of said at least one acid pH modifier, based on weight of said pharmaceutical composition.

3. The pharmaceutical composition of claim 2 wherein said at least one acid pH modifier is tartaric acid, citric acid, succinic acid, maleic acid, fumaric acid, glycolic acid, or adipic acid.

4. The pharmaceutical composition of claim 1 wherein said pharmaceutical composition is a tablet.

5. The pharmaceutical composition of claim 4, wherein said tablet comprises:
a) from about 5 to about 50 weight % of said Compound I and/or said pharmaceutically-acceptable acid salt of Compound I; and
b) from about 1 to about 50 weight % of said at least one acid pH modifier; based on weight of said pharmaceutical composition.

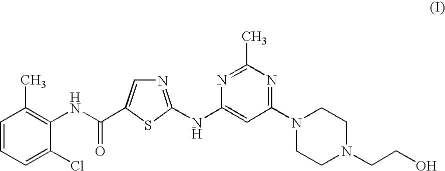

6. The pharmaceutical composition of claim 4 wherein said Compound I is in monohydrate crystalline form.

7. The pharmaceutical composition of claim 6 wherein said monohydrate crystalline form is in substantially pure form.

8. The pharmaceutical composition according to claim 1, wherein said pharmaceutically-acceptable acid salt of Compound I is a salt of: fumaric acid, hydrobromic acid, maleic acid, methanesulfonic acid, phosphoric acid, salicylic acid, sulfuric acid, tartaric acid, or p-toluenesulfonic acid.

9. The pharmaceutical composition according to claim 8 wherein said pharmaceutically-acceptable acid salt of Compound I comprises a crystalline form.

10. The pharmaceutical composition according to claim 9, wherein said pharmaceutically-acceptable acid salt of Compound I is:
methane sulfonic acid salt of Compound I comprising Form PG-1;
hydrobromic acid salt of Compound I comprising Form H1.5-1;
salicylic acid salt of Compound I comprising Form SS-2;
p-toluenesulfonic acid salt of Compound I comprising Form N-1;
D-tartaric acid salt of Compound I; and/or
L-tartaric acid salt of Compound I.

11. A method of treating cancer in a human comprising: orally administering to said human:
a) a therapeutically effective amount of Compound I of formula:
and at least one acid pH modifier (Treatment A);
and/or
b) a therapeutically effective amount of a pharmaceutically-acceptable acid salt of Compound I and one or more pharmaceutically-acceptable excipients (Treatment B).

12. The method according to claim 11, wherein said human is administered one or more medicines that raise the pH of stomach of said human prior to or during administration of Treatment A and/or Treatment B.

13. The method according to claim 11, wherein the administration of Treatment A and/or Treatment B provides enhanced bioavailability of said Compound I as compared with when said Compound I is administered unaccompanied by said at least one acid pH modifier.

14. The method according to claim 11 wherein said cancer is gastrointestinal stromal tumor (GIST) or a leukemia selected from chronic myelogenous leukemia (CML), acute lymphoblastic leukemia (ALL), Philadelphia chromosome positive acute lymphoblastic leukemia (Ph+ ALL), and acute myelogenous leukemia.

15. The method according to claim 14 wherein said cancer is a refractory cancer.

16. The method according to claim 11 comprising orally coadministrating a first dosage form comprising said Compound I and/or said pharmaceutically-acceptable acid salt of Compound I; and a second dosage form comprising said at least one acid pH modifier.

17. The method according to claim 9, wherein said Compound I or said pharmaceutically-acceptable acid salt of Compound I, is administered at an amount in the range of about 15 to 300 mg per day.

18. An acid salt comprising a salt of Compound I of formula: 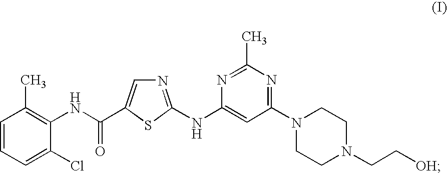
and an acid, with the proviso that said acid is not hydrochloric acid or acetic acid.

19. The acid salt according to claim 18, wherein said acid salt is substantially pure.

20. The acid salt according to claim 18, wherein said acid is: fumaric acid, hydrobromic acid, maleic acid, methanesulfonic acid, phosphoric acid, salicylic acid, sulfuric acid, tartaric acid, or p-toluenesulfonic acid.

21. The acid salt according to claim 18, wherein said acid and said Compound I are present in a 1:1 mole ratio.

22. The acid salt according to claim 21, wherein said acid salt comprises a crystalline form.

23. The acid salt according to claim 22, wherein said acid is methanesulfonic acid.

24. The acid salt according to claim 23, wherein said crystalline form is Form PG-1.

25. The acid salt according to claim 24, wherein said acid salt consists essentially of said crystalline form.

26. The acid salt according to claim 25, wherein said crystalline form is characterized by one or more of the following:
a) unit cell parameters substantially equal to the following:
Cell dimensions:
a=22.50 Å
b=8.55 Å
c=17.49 Å
α=90 degrees
β=110.7 degrees
γ=90 degrees
Space group: P2₁/a
Molecules/unit cell: 4
wherein measurement of said crystalline form is at a temperature of about −50° C.;
b) an observed powder x-ray diffraction pattern substantially as shown in FIG. 11.A;
c) a simulated powder x-ray diffraction pattern substantially as shown in FIG. 11.B; and/or
d) a powder x-ray diffraction pattern (CuKαλ=1.5418 Å) comprising four or more 2θ values selected from: 5.4±0.1, 8.2±0.1, 10.7±0.1, 11.6±0.1, 15.7±0.1, 20.6±0.1, 21.0±0.1, 23.3±0.1, and 24.4±0.1, wherein measurement of the crystalline form is at a temperature of about 25° C.

27. The acid salt according to claim 22, wherein said acid is hydrobromic acid.

28. The acid salt according to claim 27, wherein said crystalline form is Form H1.5-1.

29. The acid salt according to claim 28, wherein said acid salt consists essentially of said crystalline form.

30. The acid salt according to claim 29, wherein said crystalline form is characterized by one or more of the following:
a) unit cell parameters substantially equal to the following:
Cell dimensions:
a=7.70 Å
b=9.93 Å
c=35.23 Å
α=97.21 degrees
β=94.56 degrees
γ=91.98 degrees
Space group: Pbar1
Molecules/unit cell: 4
wherein measurement of said crystalline form is at a temperature of about −50° C.;
b) an observed powder x-ray diffraction pattern substantially as shown in FIG. 25.A;
c) a simulated powder x-ray diffraction pattern substantially as shown in FIG. 25.B; and/or
d) a powder x-ray diffraction pattern (CuKαλ=1.5418 Å) comprising four or more 2θ values selected from: 5.0±0.1, 8.9±0.1, 14.4±0.1, 17.9±0.1, 24.1±0.1, 25.1±0.1, 26.9±0.1, 28.9±0.1, and 29.3±0.1, wherein measurement of the crystalline form is at a temperature of about 25° C.

31. The acid salt according to claim 22, wherein said acid is salicylic acid.

32. The acid salt according to claim 31, wherein said crystalline form is Form SS-2.

33. The acid salt according to claim 32, wherein said acid salt consists essentially of said crystalline form.

34. The acid salt according to claim 33, wherein said crystalline form is characterized by one or more of the following:
a) unit cell parameters substantially equal to the following:
Cell dimensions:
a=22.24 Å
b=8.94 Å
c=14.87 Å
α=90 degrees
β=94.1 degrees
γ=90 degrees
Space group: P2₁/a
Molecules/unit cell: 4
wherein measurement of said crystalline form is at a temperature of about −40° C.;
b) an observed powder x-ray diffraction pattern substantially as shown in FIG. 31.A;
c) a simulated powder x-ray diffraction pattern substantially as shown in FIG. 31.B; and/or
d) a powder x-ray diffraction pattern (CuKαλ=1.5418 Å) comprising four or more 2θ values selected from: 5.9±0.1, 13.8±0.1, 14.8±0.1, 17.9±0.1, 19.8±0.1, 20.2±0.1, 23.7±0.1, and 24.8±0.1, wherein measurement of the crystalline form is at a temperature of about 25° C.

35. The acid salt according to claim 22, wherein said acid is p-toluenesulfonic acid.

36. The acid salt according to claim 35, wherein said crystalline form is Form N-1.

37. The acid salt according to claim 36, wherein said acid salt consists essentially of said crystalline form.

38. The acid salt according to claim 37, wherein said crystalline form is characterized by one or more of the following:
a) unit cell parameters substantially equal to the following:
Cell dimensions:
a=11.85 Å
b=19.04 Å
c=15.60 Å
α=90 degrees
β=116.6 degrees
γ=90 degrees
Space group: P2₁/c
Molecules/unit cell: 4
wherein measurement of said crystalline form is at a temperature of about 25° C.;
b) an observed powder x-ray diffraction pattern substantially as shown in FIG. 29.B;
c) a simulated powder x-ray diffraction pattern substantially as shown in FIG. 29.C; and/or
d) a powder x-ray diffraction pattern (CuKαλ=1.5418 Å) comprising four or more 2θ values selected from: 7.8±0.1, 8.3±0.1, 9.2±0.1, 15.7±0.1, 20.4±0.1, 22.1±0.1, 22.5±0.1, and 22.9±0.1, wherein measurement of the crystalline form is at a temperature of about 25° C.

39. The acid salt according to claim 22, wherein said acid is D-tartaric acid.

40. The acid salt according to claim 39, wherein said crystalline form is characterized by one or more of the following:
a) unit cell parameters substantially equal to the following:
Cell dimensions:
a=5.68 Å
b=11.94 Å
c=24.62 Å
α=90 degrees
β=91.7 degrees
γ=90 degrees
Space group: P2₁
Molecules/unit cell: 2
wherein measurement of said crystalline form is at a temperature of about −50° C.;
b) an observed powder x-ray diffraction pattern substantially as shown in FIG. 15.B; and/or
c) a simulated powder x-ray diffraction pattern substantially as shown in FIG. 16.B.

41. The acid salt according to claim 22, wherein said acid is L-tartaric acid.

42. The acid salt according to claim 41, wherein said crystalline form is characterized by one or more of the following:
a) unit cell parameters substantially equal to the following:
Cell dimensions:
a=5.68 Å
b=11.94 Å
c=24.62 Å
α=90 degrees
β=91.7 degrees
γ=90 degrees
Space group: P2₁
Molecules/unit cell: 2
wherein measurement of said crystalline form is at a temperature of about −50° C.;
b) an observed powder x-ray diffraction pattern substantially as shown in FIG. 16.A; and/or
c) a simulated powder x-ray diffraction pattern substantially as shown in FIG. 16.B.
